(19) United States
(12) Patent Application Publication
(10) Pub. No.: US 2013/0273113 A1
(43) Pub. Date: Oct. 17, 2013

(54) IMMUNOGENIC APOPTOSIS INDUCING COMPOSITIONS AND METHODS OF USE THEREOF

(71) Applicants: James R. Baker, JR., Ann Arbor, MI (US); Paul Makidon, Webberville, MI (US); Andrzej Myc, Ann Arbor, MI (US)

(72) Inventors: James R. Baker, JR., Ann Arbor, MI (US); Paul Makidon, Webberville, MI (US); Andrzej Myc, Ann Arbor, MI (US)

(73) Assignee: The Regents of the University of Michigan, Ann Arbor, MI (US)

(21) Appl. No.: 13/836,617

(22) Filed: Mar. 15, 2013

Related U.S. Application Data
(60) Provisional application No. 61/623,282, filed on Apr. 12, 2012.

(51) Int. Cl. A61K 39/39

(52) U.S. Cl. CPC A61K 39/39
USPC 424/278.1

(57) ABSTRACT

The present invention provides methods and compositions for the stimulation of immune responses. In particular, the present invention provides nanoemulsion compositions and methods of using the same for the induction of immune responses (e.g., immunologic cell death/apoptosis (e.g., for the induction of innate and/or adaptive immune responses)). Compositions and methods of the invention find use in, among other things, clinical (e.g. therapeutic and preventative medicine (e.g., for infectious disease, cancer, autoimmunity, and/or tissue injury (e.g., via alteration of host immune responses))) and research applications.

This application claims the benefit of and priority to U.S. Provisional Patent Application Ser. No. 61/623,282 filed 12 Apr. 2012, hereby incorporated by reference in its entirety.

This invention was made with government support under AI090031 awarded by the National Institutes of Health. The government has certain rights in the invention.

### FIELD OF THE INVENTION

The present invention provides methods and compositions for the stimulation of immune responses. In particular, the present invention provides nanoemulsion compositions and methods of using the same for the induction of immune responses (e.g., immunologic cell death/apoptosis (e.g., for the induction of innate and/or adaptive immune responses)). Compositions and methods of the invention find use in, among other things, clinical (e.g. therapeutic and preventative medicine (e.g., for infectious disease, cancer, autoimmunity, and/or tissue injury (e.g., via alteration of host immune responses))) and research applications.

### BACKGROUND

The body's immune system activates a variety of mechanisms for attacking pathogens (See, e.g., Janeway, Jr, C A. and Travers P., eds., in Immunobiology, “The Immune System in Health and Disease,” Second Edition, Current Biology Ltd., London, Great Britain (1996)). However, not all of these mechanisms are necessarily activated after immunization. Protective immunity induced by immunization is dependent upon the capacity of an immunogenic composition to elicit an appropriate immune response to resist or eliminate the pathogen. Depending on the pathogen, cell-mediated and/or humoral immune responses are important for pathogen neutralization and/or elimination.

Many antigens are poorly immunogenic or non-immunogenic when administered by themselves. Strong adaptive immune responses to antigens generally require that the antigens be administered together with an adjuvant, a substance that enhances the immune response (See, e.g., Audbert, F. M. and Lise, L. D. 1993 Immunology Today, 14: 281-284).

The need for effective therapeutic and/or preventative procedures is particularly acute with respect to infectious organisms that cause acute infections at, or gain entrance to the body through, the gastrointestinal, pulmonary, nasopharyngeal or genitourinary surfaces. Therapeutic and preventative approaches are also needed in the field of cancer biology.

### SUMMARY OF THE INVENTION

The present invention provides methods and compositions for the stimulation of immune responses. In particular, the present invention provides nanoemulsion compositions and methods of using the same for the induction of immune responses (e.g., immunologic cell death/apoptosis (e.g., for the induction of innate and/or adaptive immune responses)). Compositions and methods of the invention find use in, among other things, clinical (e.g. therapeutic and preventative medicine (e.g., for infectious disease, cancer, autoimmunity, and/or tissue injury (e.g., via alteration of host immune responses))) and research applications.

Accordingly, in one embodiment of the invention, the invention provides a method of therapeutically or prophylactically treating a condition that benefits from the induction of immunogenic apoptosis comprising administering to a mucosal surface (e.g., nasal mucosa) of a subject in need thereof an effective amount of a composition comprising a nanoemulsion. The invention is not limited by the type of condition to be treated. Indeed, a variety of conditions that will benefit from the induction of immunogenic apoptosis exist including, but not limited to, infections (e.g., bacterial, viral, fungal, yeast, etc.) and diseases such as cancer. The invention is not limited by the type of infection or the type of disease. Indeed, any condition that is therapeutically or prophylactically treatable via induction of immunogenic apoptosis will benefit from administration of a nanoemulsion as disclosed herein. For example, use of a nanoemulsion finds particular use in treating a condition wherein robust mucosal immunity, high serum antibody titers and cellular immunity that comprises both Th1 and Th17 responses are desired while concurrently avoiding the induction of histological nasal inflammation and/or epithelial disruption (e.g., no disruption of tight junctions or cell membrane). For example, in some embodiments, the nanoemulsion induces signaling pathways that activate immunogenic apoptosis/cell death (e.g., in the absence of nasal inflammation and/or epithelial disruption). In some embodiments, the nanoemulsion induces immunologic apoptosis/cell death (e.g., that induce innate and/or adaptive immune responses (e.g., not achievable with conventional, toxin based-adjuvants (e.g., CT, LT, etc.))). In some embodiments, a NE composition of the invention is used to induce immunomodulatory activities (e.g., induction of cytokine expression and/or signaling profiles) that are different from immunomodulatory activities induced by toxin based adjuvants. In some embodiments, a NE composition of the invention is used to induce antigen trafficking activities (e.g., via ciliated epithelial cells) that are different from antigen trafficking activities induced by toxin based adjuvants (e.g., via classical antigen presenting cells (e.g., macrophages and/or dendritic cells)). In some embodiments, the immunogenic apoptosis induces innate and/or adaptive immune responses. The invention is not limited by the type of innate and/or adaptive immune response induced via the immunogenic apoptosis. Indeed, a variety of immune responses can therapeutically or prophylactically treat the condition. In a preferred embodiment, the innate and/or adaptive immune responses take place in the absence of inflammation and/or epithelial disruption. In some embodiments, the innate and/or adaptive immune responses comprise mucosal innate and adaptive immune responses (e.g., including Th1 immune responses, Th17 immune responses, high avidity CD8+ cytotoxic T lymphocytes, neutralizing antibodies (e.g., neutralizing IgG1, and secretory IgA) at the site of mucosal entry, etc). In some embodiments, the immune responses comprise immunoregulatory cytokine production by ciliated nasal epithelial cells (e.g., in the absence of inflammation). In some embodiments, host DNA released from dying cells acts as a damage-associated molecular pattern (DAMP) that mediates NE adjuvant activity. In some embodiments, local and/or systemic immune responses are a result of direct interaction between NE and antigen-loaded ciliated epithelial cells and antigen-specific CD4⁺ and CD8⁺ cells in sinonasal epithelium. In other embodiments, the invention provides that epithelial cells secrete biologically active exosomes capable of uptake in DC or presenting antigenic peptide in the context of MHC class I or class II to naïve T cells. In further preferred embodiments, the nanoemulsion induces antigen uptake and trafficking via ciliated epithelial cells. In additional preferred embodiments, the antigen trafficking via ciliated epithelial cells target antigens to dendritic cells within regional and/or draining lymph nodes (e.g., targets the antigens away from the spleen). In some embodiments, the antigen targeted dendritic cells recruit lymphocytes to regional and/or draining lymph nodes and subsequent polarization toward a Th1/Th17 immune response (e.g., useful for prophylactically and/or therapeutically treating the condition). In some embodiments, polarization toward a Th1/Th17 immune response prevents the onset of infection, treats infection, prevents disease, treats disease or otherwise benefits the condition. In some embodiments, antigen-loaded ciliated epithelial cells interact directly with antigen specific lymphocytes in sinonasal epithelium (e.g., leading to beneficial local and systemic immune responses with regard to the condition). In further embodiments, DC antigen loading via epithelial cell antigen loading plays an important role in the migration of antigen loaded DCs to local, draining lymph nodes and/or to the recruitment of lymphocytes to the local, draining lymph nodes. In some embodiments, use of a NE activate apoptotic cells that possess endogenous adjuvant properties. In some embodiments, use of NE induce the exposure of heat shock proteins and/or other chaperone proteins on cellular surfaces (e.g., ciliated epithelial cells). In some embodiments, apoptotic cells induced by a NE of the invention emit and/or secrete signals that attract professional antigen presenting cells (e.g., macrophages, dendritic cells, B cells (e.g., that in turn stimulate NE specific immune responses (e.g., those disclosed herein))). In some embodiments, use of NE induces release of or surface expression of damage-associated molecular patterns (DAMPs). The invention is not limited by the type of DAMP released or expressed. For example, the DAMP may be a nucleotide product (e.g., uric acid), a nuclear and/or DNA binding protein (e.g., high-mobility group box 1 protein (HMGB1) 47, etc. In some embodiments, use of NE induces caspase activation. In some embodiments, use of NE is used to traffic antigen to local lymph nodes (e.g., away from the spleen). Although an understanding of a mechanism is not necessary to practice the invention and while the invention is not limited to any particular mechanism of action, in some embodiments, the invention provides that chemical and/or biological properties of NE (e.g., including, but not limited to, mucoadhesion properties, antigen uptake induction, toxicity or lack thereof, and/or induction of cytokine induction/secretion) are involved in the induction of innate and adaptive immune responses that occur post administration of NE to a subject. In some embodiments, immune responses comprise induction and/or expression of cytokines ganulocyte-macrophage colony-stimulating factor (GM-CSF), IL-6, IL-1a, IL-1b and MIP1α and does not comprise induction and/or expression of the cytokines IL-4 or TNF-α. In some embodiments, the induction and/or expression of cytokines occurs through activated epithelial cells. The invention is not limited by the type of nanoemulsion utilized. Indeed, a nanoemulsion may be any nanoemulsion disclosed herein. In some embodiments, the nanoemulsion comprises a positive surface charge. In some embodiments, the nanoemulsion comprises a cationic compound. The invention is not limited by the type of cationic compound. In a preferred embodiment, the cationic compound is cetylpyridinium chloride (CPC).

In an additional aspect of the invention, there is provided a method of generating an immune response in a host, including a human, comprising administering thereto an immunogenic nanoemulsion adjuvant of the present invention (e.g., independently and/or in combination with one or more antigenic (e.g., microbial pathogen (e.g., bacteria, viruses, etc.) protein, glycoprotein, lipoprotein, peptide, glycopeptide, lipopeptide, toxoid, carbohydrate, tumor-specific antigen))) components. In some embodiments, a host immune response attained via administration of a nanoemulsion adjuvant to a host subject is a humoral immune response. In some embodiments, a host immune response attained via administration of a nanoemulsion adjuvant to a host subject is a cell-mediated immune response. In some embodiments, a host immune response attained via administration of a nanoemulsion adjuvant to a host subject is an innate immune response. In some embodiments, a host immune response attained via administration of a nanoemulsion adjuvant to a host subject is a combination of innate, cell-mediated and/or humoral immune responses. In some embodiments, a composition comprising a nanoemulsion adjuvant further comprises a pharmaceutically acceptable carrier.

In some embodiments of the invention, there is provided a kit for preparing an immunogenic nanoemulsion adjuvant composition, comprising: (a) means for containing a nanoemulsion adjuvant; and (b) means for containing at least one antigen/immunogen; and (c) means for combining the nanoemulsion adjuvant and at least one antigen/immunogen to produce the immunogenic composition. The present invention provides several advantages over conventional adjuvants including, but not limited to, ease of formulation; effectiveness of adjuvanticity; lack of unwanted toxicity and/or host morbidity; and compatibility of antigens/immunogens with the adjuvant composition.

The present invention is not limited by the type of antigenic component (e.g., pathogen, pathogen component, antigen, immunogen, etc.) that can be utilized with (e.g., combined with, co-administered, administered before or after, etc.) a nanoemulsion adjuvant In certain embodiments, the antigen/immunogen is selected from the group consisting of virus, bacteria, fungus and pathogen products derived from the virus, bacteria, or fungus. The present invention is not limited to a particular virus. A variety of viral immunogens are contemplated including, but not limited to, influenza A virus, avian influenza virus, H5N1 influenza virus, H1N1 influenza virus, West Nile virus, SARS virus, Marburg virus, Arenaviruses, Nipah virus, alphaviruses, filoviruses, herpes simplex virus I, herpes simplex virus II, sendai virus, sindbis virus, vaccinia virus, parvovirus, human immunodeficiency virus, hepatitis B virus, hepatitis C virus, hepatitis A virus, cytomegalovirus, human papilloma virus, picornavirus, hantavirus, junin virus, and ebola virus. The present invention is not limited to a particular bacteria. A variety of bacterial immunogens are contemplated including, but not limited to, Bacillus cereus, Bacillus circulans and Bacillus megaterium, Bacillus anthracis, bacterial of the genus Brucella, Vibrio cholera, Coxiella burnetii, Francisella tularensis, Chlamydia psittaci, Ricinus communis, Rickettsia prowazekii, bacteria of the genus Salmonella, Cryptosporidium parvum, Burkholderia pseudomallei, Clostridium perfringens, Clostridium botulinum, Vibrio cholerae, Streptococcus pyogenes, Streptococcus agalactiae, Streptococcus pneumonia, Staphylococcus aureus, Neisseria gonorrhea, Haemophilus influenzae, Escherichia coli, Salmonella typhimurium, Shigella dysenteriae, Proteus mirabilis, Pseudomonas aeruginosa, Yersinia pestis, Yersinia enterocolitica, and Yersinia pseudotuberculosis. The present invention is also not limited to a particular fungus. A variety of fungal immunogens are contemplated including, but not limited to, Candida and Aspergillus.

In some embodiments, a nanoemulsion adjuvant provided herein skews an immune response toward a Th1 type response. In some embodiments, a nanoemulsion provided herein skews an immune response toward a Th2 type response. In some embodiments, a nanoemulsion adjuvant provided herein provides a balanced Th1/Th2 response and/or polarization (e.g., an IgG subclass distribution and cytokine response indicative of a balanced Th1/Th2 response). Thus, a variety of immune responses may be generated and/or measured in a subject administered a nanoemulsion adjuvant of the present invention including, but not limited to, activation, proliferation and/or differentiation of cells of the immune system (e.g., B cells, T cells, dendritic cells, antigen presenting cells (APCs), macrophages, natural killer (NK) cells, etc.); up-regulated or down-regulated expression of markers and/or cytokines; stimulation of IgA, IgM, and/or IgG titers; splenomegaly (e.g., increased spleen cellularity); hyperplasia, mixed cellular infiltrates in various organs, and/or other responses (e.g., of cells) of the immune system that can be assessed with respect to immune stimulation known in the art.

In some embodiments, administering comprises contacting a mucosal surface of the subject with the adjuvant. The invention is not limited by the mucosal surface contacted. In some preferred embodiments, the mucosal surface comprises nasal mucosa. In some embodiments, the mucosal surface comprises vaginal mucosa. In some embodiments, administrating comprises parenteral administration. The present invention is not limited by the route chosen for administration of an adjuvant of the present invention. In some embodiments, inducing an immune response primes the immune system of a host to respond to (e.g., to produce a Th1 and/or Th2 type response (e.g., thereby providing protective immunity) one or more pathogens in the host subject (e.g., human or animal subject). In some embodiments, the immunity comprises systemic immunity. In some embodiments, the immunity comprises mucosal immunity. In some embodiments, the immune response comprises increased expression of IFN-γ and/or TNF-α in the subject. In some embodiments, the immune response comprises a systemic IgG response. In some embodiments, the immune response comprises a mucosal IgA response.

In some embodiments, the invention provides an immunogenic composition for eliciting an immune response in a host, including a human, the composition comprising: (a) at least one antigen and/or immunogen; and (b) a nanoemulsion adjuvant. In some embodiments, the composition comprises an additional adjuvant (e.g., a second nanoemulsion adjuvant and/or a non-nanoemulsion adjuvant (e.g., CpG oligonucleotide, toxin, or other adjuvant described herein).

In some embodiments, the invention provides a method of stimulating immunogenic apoptosis in a subject in need thereof comprising administering (e.g., to a mucosal surface) to the subject an effective amount of a composition comprising a nanoemulsion to induce immunogenic apoptosis. In some embodiments, inducing immunogenic apoptosis in a subject comprises the activation, induction, stimulation and/or augmentation of caspase 8 activity. In some embodiments, activation, induction, stimulation and/or augmentation of caspase 8 activity results in calreticulin expression in the subject (e.g., in the nasal mucosa of the subject). The invention is not limited by the type of subject that would benefit from the induction of immunogenic apoptosis. Indeed, a variety of subjects will benefit from the induction of immunogenic apoptosis via administration of an effective amount of a nanoemulsion described herein including, but not limited to, a subject with cancer, a subject with a wound, and a subject with fibrosis (See, e.g., Gold et al. FASEB 2010; 24(3), 665-683).

In yet another aspect of the invention, there is provided a method of modulating and/or inducing an immune response (e.g., toward and/or away from a Th1 and/or Th2 type response) in a subject (e.g., toward an antigen) comprising providing a host subject and a nanoemulsion adjuvant composition of the invention, and administering the nanoemulsion adjuvant to the host subject under conditions such that an immune response is induced and/or modulated in the host subject. In some embodiments, the host immune response is specific for the nanoemulsion adjuvant. In some embodiments, the host immune response comprises enhanced expression and/or activity of Th1 type cytokines (e.g., IL-2, IL-12, IFN-γ and/or TNF-α, etc.) while concurrently lacking enhanced expression and/or activity of Th2 type cytokines (e.g., IL-4, IL-5, IL-10, etc.). In some embodiments, the host immune response comprises enhanced expression of Th2 type cytokines (e.g., IL-4, IL-5, IL-10, etc.) while concurrently lacking enhanced expression and/or activity of Th1 type cytokines (e.g., (e.g., IL-2, IL-12, IFN-γ and/or TNF-α, etc.). In some embodiments, a nanoemulsion adjuvant composition administered to a subject induces expression and/or activity of Th1-type cytokines that increases to a greater extent than the level of expression and/or activity of Th2-type cytokines. For example, in some embodiments, a subject administered a nanoemulsion adjuvant composition induces a greater than 3 fold, greater than 5 fold, greater than 10 fold, greater than 20 fold, greater than 25 fold, greater than 30 fold or more enhanced expression of Th1 type cytokines (e.g., IL-2, IL-12, IFN-γ and/or TNF-α), with lower increases (e.g., less than 3 fold, less than two fold or less) enhanced expression of Th2 type cytokines (e.g., IL-4, IL-5, and/or IL-10). In some embodiments, a nanoemulsion adjuvant composition administered to a subject induces expression and/or activity of Th2-type cytokines that increases to a greater extent than the level of expression and/or activity of Th1-type cytokines. For example, in some embodiments, a subject administered a nanoemulsion adjuvant composition induces a greater than 3 fold, greater than 5 fold, greater than 10 fold, greater than 20 fold, greater than 25 fold, greater than 30 fold or more enhanced expression of Th2 type cytokines (e.g., IL-4, IL-5, and/or IL-10), with lower increases (e.g., less than 3 fold, less than two fold or less) enhanced expression of Th1 type cytokines (e.g., IL-2, IL-12, IFN-γ and/or TNF-α). In some embodiments, the host immune response comprises enhanced IL6 cytokine expression and/or activity while concurrently lacking enhanced expression and/or activity of other cytokines (e.g., IL4, TNF-α and/or IFN-γ) in the host. In some embodiments, the host immune response is specific for an antigen co-administered with the nanoemulsion adjuvant. In some embodiments, administering the nanoemulsion adjuvant to the host subject (e.g., in combination with an antigenic component (e.g., whole cell pathogen or component thereof)) induces and/or enhances the generation of one or more antibodies in the subject (e.g., IgG and/or IgA antibodies) that are not generated or generated at low levels in the host subject in the absence of administration of the nanoemulsion adjuvant. In some embodiments, administering the nanoemulsion adjuvant to the host induces a specific response to the nanoemulsion adjuvant by epithelial cells of the host. In some embodiments, administering the nanoemulsion adjuvant to the host induces uric acid and/or inflamasome activation in the host (e.g., that is distinguishable from uric acid and/or inflamasome activation induced by other types of adjuvants (e.g., alum adjuvants).

In some embodiments, host immune responses resulting from administration of a nanoemulsion adjuvant (e.g., individually and/or in combination with an antigenic/immunogenic component (e.g., whole cell pathogen or component thereof)) protects a subject from challenge with a subsequent exposure to live pathogen. In some embodiments, a nanoemulsion adjuvant further comprises one or more additional adjuvants. The present invention is not limited by the type of additional adjuvant utilized. In some embodiments, the additional adjuvant is a CpG oligonucleotide. In some embodiments, the additional adjuvant is monophosphoryl lipid A. A number of other adjuvants that find use in the present invention are described herein. In some embodiments, the subject is a human. In some embodiments, immune responses resulting from administration of a nanoemulsion adjuvant (e.g., individually and/or in combination with immunogenic pathogen components) reduces the risk of infection upon one or more exposures to a pathogen. In some embodiments, administration of a nanoemulsion adjuvant to a host subject (e.g., in combination with an antigenic component (e.g., whole cell pathogen or component thereof)) induces the generation of one or more antibodies in the subject (e.g., IgG and/or IgA antibodies) that are not generated in the host subject in the absence of administration of the nanoemulsion adjuvant.

### BRIEF DESCRIPTION OF THE DRAWINGS

The following figures form part of the present specification and are included to further demonstrate certain aspects and embodiments of the present invention. The invention may be better understood by reference to one or more of these figures in combination with the description of specific embodiments presented herein.

FIG. 1 shows a comparison of nanoemulsion versus cholera toxin (CT) adjuvant activities in vivo. B6.Cg-Tg(HLA-A/H2-D)2Enge/J mice were intranasally immunized with either 20% NE (poloxamer 470-based)+20 μg HBsAg, 1 μg CT+20 μg HBsAg or 20 μg HBsAg alone 3 times four weeks apart. (A) Serum anti-HBsAg evaluated via ELISA. The antibody concentrations are presented as endpoint titers defined as the reciprocal of the highest serum dilution producing an OD₄₅₀ₙₘ above cutoff value. The cutoff value is determined as OD₄₅₀ₙₘ of the corresponding dilution of control sera plus 2 (standard deviations) and plate background. The error bars indicate standard error measurement. (B) Anti-HBsAg IgA antibody measured via ELISA using bronchial lavage (BAL) collected post-mortem from immunized mice. The results are expressed as measurements of OD₄₅₀ₙₘ. The BAL was analyzed undiluted and represents an infusion and aspiration on 1600 μl of fluid representing a dilution of 1:1600. Data were analyzed by unpaired Student's t-test.

FIG. 2 shows that anoemulsion promotes mucosal antigen uptake and trafficking to regional lymphoid tissue. (A) Nanoemulsion enhanced in vivo trafficking of co-mixed QDOTs to regional lymphoid tissue following nasal administration. Mice treated with QDOTs mixed with NE (upper left), QDOTs in PBS (upper middle) or 20% NE only (upper right). In vivo fluorescence was measured with an IVIS Imaging System 200 Spectrum series bioluminometer at 18 hours. The fifth mouse in each group is a non-treated control (mouse to the farthest right in each photo). QDOT-specific fluorescent intensity is represented on an increasing scale from blue (1×10⁷), green (5×10⁷), yellow, (7.5×10⁷), and red (1×10⁸) photons/sec/cm²/sr, respectively. The fluorescent measurement was quantified using IVIS Living Image 3.1 software (Caliper Life Sciences, Hopkinton, Mass.) in three regions overlying the nose, the cervical LN and the mediastinal LN as indicated. The fluorescent intensity for each region was normalized to the signal collected from the blank on each mouse. Group average (±SEM) quantified results (y-axis) are presented as the ratio of the quantified fluorescence from the identified quantification area/the quantified result from the whole body of each individual mouse for each time point of measurement x-axis). The graph shows quantified group-average results for nose, cervical LN and mediastinal LN. “*” indicates significant (p<0.05) difference in the normalized quantified fluorescence for NE-QDOT treated mice in comparison to QDOT only treated mice. Mouse #2 in the NE only treatment group died under anesthesia during the 4 hour imaging time point and was not included in further measurements. (B) NE-facilitated in vivo uptake and lymphoid distribution of GFP. In situ fluorescent evaluation of cryo-preserved nasal epithelium in CD-1 mice 18 hours following instillation of 7.5 μl/nare of GFP (10 μg) diluted in PBS (Upper left) or mixed with 20% NE (Lower left). Photomicrographs of cervical LN (Upper middle) & (Lower middle) and mediastinal LN (Upper right) & (Lower right) tissues of GFP and GFP-NE mice, respectively. Images presented at 400× magnification using an epiflourescent microscope. (C) NE enhanced antigen uptake in NALT. In situ fluorescent evaluation of NALT in CD-1 mice 18 hours following instillation of 6 μl/nare of GFP (10 μg). Naïve mice (Photomicrograph upper left), Mice treated with GFP in PBS (Photomicrograph upper right), Mice treated with 1 μg CT mixed with GFP (Photomicrograph lower left) or mice treated with 20% NE mixed with GFP (Photomicrograph lower right). Tissues were probed with anti-GFP antibody. Images presented at 400× magnification using laser confocal microscopy. Insets in FIGS. B and D represent zoom magnification of selected areas. “*” and the dashed white line demark the area of the sub-epithelial dome. (D) FACS analysis of OVA-Alexa 647 uptake in NALT tissues. Single cell suspension of NALT cells (1−2×10⁶/sample) isolated from CD-1 mice 36 hours following nasal treatment with 10 μg OVA-Alexa 647±20% NE (10 μl/nare) or 20% NE alone (10 μl/nare).

Numbers represent the percentage of cells that have internalized OVA-Alexa 647 among CD11c⁺ lymphocytes. (E) Group average population NALT derived of antigen-loaded CD11c⁺±STD. “*” indicates statistically significant change in the percentage of antigen-loaded cells isolated from mice treated with OVA plus NE versus those treated with OVA only. Data were analyzed by unpaired Student's t-test.

FIG. 3 shows that NE promotes in vivo sampling by ciliated nasal epithelial cells without disruption of the epithelial barrier. TEM images of nasal epithelium from CD-1 mice 18 hours after nasal inoculation with QDOTs: images of epithelium following 20% NE treatment (7.5 μl/nare) at various magnifications (Photomicrographs in the top row). Arrows in the upper right hand photomicrograph point to tight junctions between adjacent epithelial cells. Epithelium following intranasal inoculation with QDOTs mixed with 20% NE (The photomicrograph in the lower left hand corner) (7,900× magnification) and (The middle photomicrograph in the lower row) (130,000× magnification). Arrows in (The photomicrograph in the lower left hand corner) point to the basal lamina. The vesicle-like structure containing aggregates of QDOTs (The middle photomicrograph in the lower row) has an average diameter of 0.455 microns. The QDOT-like material present in the vesicle-like structure measured on average 5 nm. (The photomicrograph in the lower right hand corner is an image of control (non-treated) nasal epithelium. Data were analyzed by unpaired Student's t-test.

FIG. 4 shows NE stimulates immunogenic epithelial cell apoptosis and necrosis. (A) Evaluation of apoptosis and necrosis in vivo. Nasal respiratory epithelium was harvested from 10 week old female C57BL/6N mice 2 hours following treatment with 15 μl 20% NE (Lower left) or its components CPC (Upper right) or nanoemulsion without CPC (W₈₀5E) (Lower right). The tissue was fixed in buffered formalin and stained for the apoptotic marker caspase-3. Cells staining with caspase-3 appear to have dark brown inclusions (red arrows). These results were compared to PBS negative treatment control mice (Upper left). Necrotic cell death was evaluated morphologically. Necrotic cells contain dilated organelles and dissociated ribosomes from the endoplasmic reticulum (black arrows). These cells do not contain pyknotic or fragmented nuclei and the degeneration proceeds without any detectable involvement of lysosomes. Neutrophilic inflammation (green arrows) is observed in CPC alone treated groups. Sections were imaged at 400×. (B) Evaluation of dying cells for surface expression of immunogenic apoptotic marker calreticulin. Epithelial cells expressing calreticulin appear dark in color (lower left). Sections were imaged at 200×.

FIG. 5 shows NE-modulated MHC class I and class II gene expression in nasal mucosa (A) NE-modulated MHC class I and class II gene expression in nasal mucosa. Hierarchal cluster analysis of antigen processing and presentation pathway gene expression in CD-1 mouse nasal mucosa 6 or 24 hours following exposure to 20% NE (15 μl). Gene regulating expression of MHC class I or class II is indicated by the light or dark arrows, respectively. The colors represent significant (p<0.05 and fold change >2 over control tissue) changes in gene expression (dark/red=up-regulated and light/green=down-regulated). (B) MHC class II surface expression in primary nasal epithelial cells. 1×10⁶ nasal epithelial cells harvested from C57BL/6N mice were treated with either 0.0001% NE, 10 μg CT, or media alone for 12 hours. The cells were probed for MHC II expression and analyzed via flow cytometry. MHC II expression on primary epithelial cells untreated (media alone) (black histogram), NE treated cells (light grey histogram), and CT treated cells (dark grey histogram).

FIG. 6 shows NE adjuvant promotes in vivo GFP localization in cells expressing DEC205. Co-localization of GFP and DEC205 surface markers in the cervical LN of CD-1 mice 18 h after nasal treatment with 13 μg GFP plus 20% NE (15 μl). The photomicrograph in the upper left corner represents green channel fluorescence (GFP). The photomicrograph in the upper right corner represents red channel fluorescence (DEC205). The photomicrograph in the lower left corner represents ultraviolet channel fluorescence (DAPI). The photomicrograph in the lower right corner represents the overlay image of all of the channels.

FIG. 7 shows that nanoemulsion has unique immunomodulatory function in respiratory mucosa. (A) Immunodetection of NE-driven mucosally secreted innate cytokines and chemokines after intranasal treatment with NE. Cytokine and chemokine secretion as detected by Luminex Multiplex22 assay in homogenized nasal septal tissues collected from C57BL/6N mice 18 hours following intranasal administration of 20% NE (7.5 μl/nare), or 1 μg CT (7.5 μl/nare), or PBS (7.5 μl/nare). TGF-β and TSLP were measured by ELISA. Protein detection is expressed in protein concentration pg/ml. (B) Immunodetection of cytokines and chemokines in bone marrow derived dendritic cells (BMDC). 4×10⁶ BMDCs were stimulated with 0.001%, or 0.01% or 0.1% of nanoemulsion, or 1 μg/ml, or 10 μg/ml or 30 μg/ml of cholera toxin for 24 hours. Cytokine secretion was measured in supernatant using Multiplex22. Protein detection is expressed in protein concentration pg/ml. (C) Nanomeulsion mediates a unique cytokine profile and requires the participation of stromal cells. VEN diagram comparing the NE-specific versus CT-specific profiles of cytokines and chemokines in nasal mucosa (Top) versus BMDC (Bottom). Supernatant from TC-1 cells treated with 0.001%, or 0.01% or 0.1% of NE, or 1 μg/ml, or 10 μg/ml or 30 μg/ml of cholera toxin were also evaluated for IL-6, TGF-β and TSLP by ELISA. “*” indicates validated NE-specific production in TC-1 epithelial cells. Data were analyzed by unpaired Student's t-test.

FIG. 8 shows caspase 8 activation increases along with increased concentrations of NE. At higher concentrations of NE>0.045% NE becomes cytotoxic to the cells and kills cells directly (e.g., via lysis and/or necrosis).

### DEFINITIONS

To facilitate an understanding of the present invention, a number of terms and phrases are defined below:

As used herein, the term “microorganism” refers to any species or type of microorganism, including but not limited to, bacteria, viruses, archaea, fungi, protozoans, mycoplasma, prions, and parasitic organisms. The term microorganism encompasses both those organisms that are in and of themselves pathogenic to another organism (e.g., animals, including humans, and plants) and those organisms that produce agents that are pathogenic to another organism, while the organism itself is not directly pathogenic or infective to the other organism.

As used herein the term “pathogen,” and grammatical equivalents, refers to an organism (e.g., biological agent), including microorganisms, that causes a disease state (e.g., infection, pathologic condition, disease, etc.) in another organism (e.g., animals and plants) by directly infecting the other organism, or by producing agents that causes disease in another organism (e.g., bacteria that produce pathogenic toxins and the like). “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc.

As used herein, the term “fungi” is used in reference to eukaryotic organisms such as molds and yeasts, including dimorphic fungi.

As used herein the terms “disease” and “pathologic condition” are used interchangeably, unless indicated otherwise herein, to describe a deviation from the condition regarded as normal or average for members of a species or group (e.g., humans), and which is detrimental to an affected individual under conditions that are not inimical to the majority of individuals of that species or group. Such a deviation can manifest as a state, signs, and/or symptoms (e.g., diarrhea, nausea, fever, pain, blisters, boils, rash, immune suppression, inflammation, etc.) that are associated with any impairment of the normal state of a subject or of any of its organs or tissues that interrupts or modifies the performance of normal functions. A disease or pathological condition may be caused by or result from contact with a microorganism (e.g., a pathogen or other infective agent (e.g., a virus or bacteria)), may be responsive to environmental factors (e.g., malnutrition, industrial hazards, and/or climate), may be responsive to an inherent defect of the organism (e.g., genetic anomalies) or to combinations of these and other factors.

The terms “host” or “subject,” as used herein, refer to an individual to be treated by (e.g., administered) the compositions and methods of the present invention. Subjects include, but are not limited to, mammals (e.g., murines, simians, equines, bovines, porcines, canines, felines, and the like), and most preferably includes humans. In the context of the invention, the term “subject” generally refers to an individual who will be administered or who has been administered one or more compositions of the present invention (e.g., a nanoemulson composition (e.g., for inducing immunogenic apoptosis (e.g. for inducing innate and/or adaptive immune responses)).

As used herein, the terms “inactivating,” “inactivation” and grammatical equivalents, when used in reference to a microorganism (e.g., a pathogen (e.g., a bacterium or a virus)), refer to the killing, elimination, neutralization and/or reducing of the capacity of the microorganism (e.g., a pathogen (e.g., a bacterium or a virus)) to infect and/or cause a pathological response and/or disease in a host. For example, in some embodiments, the present invention provides a composition comprising nanoemulsion (NE)-inactivated vaccinia virus (VV). Accordingly, as referred to herein, compositions comprising “NE-inactivated VV,” “NE-killed V,” NE-neutralized V” or grammatical equivalents refer to compositions that, when administered to a subject, are characterized by the absence of, or significantly reduced presence of, VV replication (e.g., over a period of time (e.g., over a period of days, weeks, months, or longer)) within the host.

As used herein, the term “fusigenic” is intended to refer to an emulsion that is capable of fusing with the membrane of a microbial agent (e.g., a bacterium or bacterial spore). Specific examples of fusigenic emulsions are described herein.

As used herein, the term “lysogenic” refers to an emulsion (e.g., a nanoemulsion) that is capable of disrupting the membrane of a microbial agent (e.g., a virus (e.g., viral envelope) or a bacterium or bacterial spore). In preferred embodiments of the present invention, the presence of a lysogenic and a fusigenic agent in the same composition produces an enhanced inactivating effect compared to either agent alone. Methods and compositions (e.g., for inducing an immune response (e.g., used as a vaccine) using this improved antimicrobial composition are described in detail herein.

The term “emulsion,” as used herein, includes classic oil-in-water or water in oil dispersions or droplets, as well as other lipid structures that can form as a result of hydrophobic forces that drive apolar residues (e.g., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. Similarly, the term “nanoemulsion,” as used herein, refers to oil-in-water dispersions comprising small lipid structures. For example, in some embodiments, the nanoemulsions comprise an oil phase having droplets with a mean particle size of approximately 0.1 to 5 microns (e.g., about 150, 200, 250, 300, 350, 400, 450, 500 nm or larger in diameter), although smaller and larger particle sizes are contemplated. The terms “emulsion” and “nanoemulsion” are often used herein, interchangeably, to refer to the nanoemulsions of the present invention.

As used herein, the terms “contact,” “contacted,” “expose,” and “exposed,” when used in reference to a nanoemulsion and a live microorganism, refer to bringing one or more nanoemulsions into contact with a microorganism (e.g., a pathogen) such that the nanoemulsion inactivates the microorganism or pathogenic agent, if present. The present invention is not limited by the amount or type of nanoemulsion used for microorganism inactivation. A variety of nanoemulsion that find use in the present invention are described herein and elsewhere (e.g., nanoemulsions described in U.S. Pat. Apps. 20020045667 and 20040043041, and U.S. Pat. Nos. 6,015,832, 6,506,803, 6,635,676, and 6,559,189, each of which is incorporated herein by reference in its entirety for all purposes). Ratios and amounts of nanoemulsion (e.g., sufficient for inactivating the microorganism (e.g., virus inactivation)) and microorganisms (e.g., sufficient to provide an antigenic composition (e.g., a composition capable of inducing an immune response)) are contemplated in the present invention including, but not limited to, those described herein.

The term “surfactant” refers to any molecule having both a polar head group, which energetically prefers solvation by water, and a hydrophobic tail that is not well solvated by water. The term “cationic surfactant” refers to a surfactant with a cationic head group. The term “anionic surfactant” refers to a surfactant with an anionic head group.

The terms “Hydrophile-Lipophile Balance Index Number” and “HLB Index Number” refer to an index for correlating the chemical structure of surfactant molecules with their surface activity. The HLB Index Number may be calculated by a variety of empirical formulas as described, for example, by Meyers, (See, e.g., Meyers, Surfactant Science and Technology, VCH Publishers Inc., New York, pp. 231-245 (1992)), incorporated herein by reference. As used herein where appropriate, the HLB Index Number of a surfactant is the HLB Index Number assigned to that surfactant in McCutcheon's Volume 1: Emulsifiers and Detergents North American Edition, 1996 (incorporated herein by reference). The HLB Index Number ranges from 0 to about 70 or more for commercial surfactants. Hydrophilic surfactants with high solubility in water and solubilizing properties are at the high end of the scale, while surfactants with low solubility in water that are good solubilizers of water in oils are at the low end of the scale.

As used herein the term “interaction enhancers” refers to compounds that act to enhance the interaction of an emulsion with a microorganism (e.g., with a cell wall of a bacteria (e.g., a Gram negative bacteria) or with a viral envelope (e.g., Vaccinia virus envelope)). Contemplated interaction enhancers include, but are not limited to, chelating agents (e.g., ethylenediaminetetraacetic acid (EDTA), ethylenebis(oxyethylenenitrilo)tetraacetic acid (EGTA), and the like) and certain biological agents (e.g., bovine serum albumin (BSA) and the like).

The terms “buffer” or “buffering agents” refer to materials, that when added to a solution, cause the solution to resist changes in pH.

The terms “reducing agent” and “electron donor” refer to a material that donates electrons to a second material to reduce the oxidation state of one or more of the second material's atoms.

The term “monovalent salt” refers to any salt in which the metal (e.g., Na, K, or Li) has a net 1+ charge in solution (i.e., one more proton than electron).

The term “divalent salt” refers to any salt in which a metal (e.g., Mg, Ca, or Sr) has a net 2+ charge in solution.

The terms “chelator” or “chelating agent” refer to any materials having more than one atom with a lone pair of electrons that are available to bond to a metal ion.

The term “solution” refers to an aqueous or non-aqueous mixture.

As used herein, the terms “a composition for inducing immunologic cell death” and “a composition for inducing immunologic apoptosis” refer to a composition that, once administered to a subject (e.g., once, twice, three times or more (e.g., separated by weeks, months or years)), stimulates, generates and/or elicits immune responses and/or signals that emanate from and/or that are associated with dying cells (e.g. that promote innate and/or adaptive immune responses (e.g., directed at infectious disease organisms/immunogens and/or cancer/tumors)) in a host administered the composition (e.g., resulting in total or partial immunity to a microorganism (e.g., pathogen) or tumor that is capable of causing disease).

As used herein, the term “a composition for inducing an immune response” refers to a composition that, once administered to a subject (e.g., once, twice, three times or more (e.g., separated by weeks, months or years)), stimulates, generates and/or elicits an immune response in the subject (e.g., resulting in total or partial immunity to a microorganism (e.g., pathogen) capable of causing disease). In preferred embodiments of the invention, the composition comprises a nanoemulsion. In a further preferred embodiments of the invention, the composition comprises a nanoemulsion and one or more immunogens. In further preferred embodiments, a composition comprising a nanoemulsion and an immunogen comprises one or more other compounds or agents including, but not limited to, therapeutic agents, physiologically tolerable liquids, gels, carriers, diluents, adjuvants, excipients, salicylates, steroids, immunosuppressants, immunostimulants, antibodies, cytokines, antibiotics, binders, fillers, preservatives, stabilizing agents, emulsifiers, and/or buffers. An immune response may be an innate (e.g., a non-specific) immune response or an adaptive/learned (e.g., acquired) immune response (e.g. that decreases the infectivity, morbidity, or onset of mortality in a subject (e.g., caused by exposure to a pathogenic microorganism) or that prevents infectivity, morbidity, or onset of mortality in a subject (e.g., caused by exposure to a pathogenic microorganism)). Thus, in some preferred embodiments, a composition comprising a nanoemulsion and an immunogen is administered to a subject as a vaccine (e.g., to prevent or attenuate a disease (e.g., by providing to the subject total or partial immunity against the disease or the total or partial attenuation (e.g., suppression) of a sign, symptom or condition of the disease (e.g., infectious disease or cancer).

As used herein, the term “adjuvant” refers to a substance that can stimulate an immune response (e.g., innate and/or adaptive immune response (e.g., a mucosal immune response)). Some adjuvants can cause activation of a cell of the immune system (e.g., an adjuvant can cause an immune cell to produce and secrete a cytokine). Examples of adjuvants that can cause activation of a cell of the immune system include, but are not limited to, a nanoemulsion formulations described herein, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”). In some embodiments, compositions comprising nanoemulsion of the invention are administered alone or with one or more adjuvants to a subject (e.g., in order to induce immunologic apoptosis (e.g., to induce innate and/or adaptive immune responses (e.g., to skew a host's immune response towards a Th1, Th2, or Th17 type immune response).

As used herein, the term “effective amount” for example, as in “an effective amount to induce an immune response” (e.g., of a composition for inducing an immune response), refers to the amount or dosage level required (e.g., when administered to a subject) to have a desired effect (e.g., to stimulate, generate and/or elicit an immune response in a subject). An effective amount can be administered in one or more administrations (e.g., via the same or different route), applications or dosages and is not intended to be limited to a particular formulation or administration route.

As used herein, the term “under conditions such that said subject generates an immune response” refers to any qualitative or quantitative induction, generation, and/or stimulation of an immune response (e.g., innate or adaptive/acquired).

A used herein, the term “immune response” refers to a response by the immune system of a subject. For example, immune responses include, but are not limited to, a detectable alteration (e.g., increase) in Toll-like receptor (TLR) activation, lymphokine (e.g., cytokine (e.g., Th1, Th2 or Th17 type cytokines) or chemokine) expression and/or secretion, macrophage activation, dendritic cell activation, T cell activation (e.g., CD4+ or CD8+ T cells), NK cell activation, B cell activation (e.g., antibody generation and/or secretion), immunogenic cell death/apoptosis, altered (e.g., enhanced) antigenicity of dying cells, caspase expression and/or activity, immune cell recruitment, induction of heat shock protein expression and/or translocation to cell surfaces, alteration (e.g., elevation) of cellular signaling molecules (e.g., damage-associated molecular patterns (DAMPS) (e.g., lysophosphatidylcholine, oligonucleotides, nucleosides, urate)), and/or altered (e.g., enhanced) engulfment of apoptotic bodies, antigen processing (e.g. via non-classical antigen presenting cells (e.g. epithelial cell antigen uptake (e.g. ciliated nasal epithelial cells)), dendritic cell maturation, and/or T cell activation.

Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the terms “toll receptors” and “TLRs” refer to a class of receptors (e.g., TLR1, TLR2, TLR3, TLR4, TLR5, TLR6, TLR7, TLR8, TLR9, TLRT0, TLR 11) that recognize special patterns of pathogens, termed pathogen-associated molecular patterns (See, e.g., Janeway and Medzhitov, (2002) Annu Rev. Immunol. 20, 197-216). These receptors are expressed in innate immune cells (e.g., neutrophils, monocytes, macrophages, dendritic cells) and in other types of cells such as endothelial cells. Their ligands include bacterial products such as LPS, peptidoglycans, lipopeptides, and CpG DNA. TLRs are receptors that bind to exogenous ligands and mediate innate immune responses leading to the elimination of invading microbes. The TLR-triggered signaling pathway leads to activation of transcription factors including NFkB, which is important for the induced expression of proinflammatory cytokines and chemokines TLRs also interact with each other. For example, TLR2 can form functional heterodimers with TLR1 or TLR6. The TLR2/1 dimer has different ligand binding profile than the TLR2/6 dimer (Ozinsky et al., 2000). In some embodiments, a nanoemulsion adjuvant activates cell signaling through a TLR (e.g., TLR2 and/or TLR4). Thus, methods described herein include a nanoemulsion adjuvant composition (e.g., composition comprising NE adjuvant optionally combined with one or more immunogens (e.g., proteins and/or NE adjuvant inactivated pathogen (e.g., a virus (e.g., VV)))) that when administered to a subject, activates one or more TLRs and stimulates an immune response (e.g., innate and/or adaptive/acquired immune response) in a subject. Such an adjuvant can activate TLRs (e.g., TLR2 and/or TLR4) by, for example, interacting with TLRs (e.g., NE adjuvant binding to TLRs) or activating any downstream cellular pathway that occurs upon binding of a ligand to a TLR. NE adjuvants described herein that activate TLRs can also enhance the availability or accessibility of any endogenous or naturally occurring ligand of TLRs. A NE adjuvant that activates one or more TLRs can alter transcription of genes, increase translation of mRNA or increase the activity of proteins that are involved in mediating TLR cellular processes. For example, NE adjuvants described herein that activate one or more TLRs (e.g., TLR2 and/or TLR4) can induce expression of one or more cytokines (e.g., IL-8, IL-12p40, and/or IL-23).

As used herein, the term “immunity” refers to protection from disease (e.g., preventing or attenuating (e.g., suppression) of a sign, symptom or condition of the disease) upon exposure to a microorganism (e.g., pathogen) capable of causing the disease. Immunity can be innate (e.g., non-adaptive (e.g., non-acquired) immune responses that exist in the absence of a previous exposure to an antigen) and/or acquired/adaptive (e.g., immune responses that are mediated by B and T cells following a previous exposure to antigen (e.g., that exhibit increased specificity and reactivity to the antigen)).

As used herein, the terms “immunogen” and “antigen” refer to an agent (e.g., a microorganism (e.g., bacterium, virus or fungus) and/or portion or component thereof (e.g., a protein antigen (e.g., gp120 or rPA)) or a part or portion of a cancer/tumor) that is capable of eliciting an immune response in a subject. In preferred embodiments, immunogens elicit innate and/or adaptive immune responses against the immunogen (e.g., microorganism (e.g., pathogen or a pathogen product) or cancer/tumor) when administered in combination with a nanoemulsion of the present invention.

As used herein, the term “pathogen product” refers to any component or product derived from a pathogen including, but not limited to, polypeptides, peptides, proteins, nucleic acids, membrane fractions, and polysaccharides.

As used herein, the term “enhanced immunity” refers to an increase in the level of adaptive and/or acquired immunity in a subject to a given immunogen (e.g., microorganism (e.g., pathogen)) following administration of a composition (e.g., composition for inducing an immune response of the present invention) relative to the level of adaptive and/or acquired immunity in a subject that has not been administered the composition (e.g., composition for inducing an immune response of the present invention).

As used herein, the terms “purified” or “to purify” refer to the removal of contaminants or undesired compounds from a sample or composition. As used herein, the term “substantially purified” refers to the removal of from about 70 to 90%, up to 100%, of the contaminants or undesired compounds from a sample or composition.

As used herein, the terms “administration” and “administering” refer to the act of giving a composition of the present invention (e.g., a composition for inducing an immune response (e.g., a composition comprising a nanoemulsion)) to a subject. Exemplary routes of administration to the human body include, but are not limited to, through the eyes (ophthalmic), mouth (oral), skin (transdermal), nose (nasal), lungs (inhalant), oral mucosa (buccal), ear, rectal, by injection (e.g., intravenously, subcutaneously, intraperitoneally, etc.), topically, and the like.

As used herein, the terms “co-administration” and “co-administering” refer to the administration of at least two agent(s) (e.g., a composition comprising a nanoemulsion a and one or more other agents—e.g., an immunogen and/or antigen) or therapies to a subject. In some embodiments, the co-administration of two or more agents or therapies is concurrent. In other embodiments, a first agent/therapy is administered prior to a second agent/therapy. In some embodiments, co-administration can be via the same or different route of administration. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent. In other embodiments, co-administration is preferable to elicit an immune response in a subject to two or more different immunogens (e.g., microorganisms (e.g., pathogens)) at or near the same time (e.g., when a subject is unlikely to be available for subsequent administration of a second, third, or more composition for inducing an immune response).

As used herein, the term “topically” refers to application of a compositions of the present invention (e.g., a composition comprising a nanoemulsion and an immunogen) to the surface of the skin and/or mucosal cells and tissues (e.g., alveolar, buccal, lingual, masticatory, vaginal or nasal mucosa, and other tissues and cells which line hollow organs or body cavities).

In some embodiments, the compositions of the present invention are administered in the form of topical emulsions, injectable compositions, ingestible solutions, and the like. When the route is topical, the form may be, for example, a spray (e.g., a nasal spray), a cream, or other viscous solution (e.g., a composition comprising a nanoemulsion and an immunogen in polyethylene glycol).

The terms “pharmaceutically acceptable” or “pharmacologically acceptable,” as used herein, refer to compositions that do not substantially produce adverse reactions (e.g., toxic, allergic or immunological reactions) when administered to a subject.

As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintrigrants (e.g., potato starch or sodium starch glycolate), polyethylethe glycol, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a composition of the present invention that is physiologically tolerated in the target subject. “Salts” of the compositions of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compositions of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compositions of the present invention are contemplated as being pharmaceutically acceptable. However, salts of acids and bases that are non-pharmaceutically acceptable may also find use, for example, in the preparation or purification of a pharmaceutically acceptable composition.

As used herein, the term “at risk for disease” refers to a subject that is predisposed to experiencing a particular disease. This predisposition may be genetic (e.g., a particular genetic tendency to experience the disease, such as heritable disorders), or due to other factors (e.g., environmental conditions, exposures to detrimental compounds present in the environment, etc.). Thus, it is not intended that the present invention be limited to any particular risk (e.g., a subject may be “at risk for disease” simply by being exposed to and interacting with other people), nor is it intended that the present invention be limited to any particular disease.

“Nasal application”, as used herein, means applied through the nose into the nasal or sinus passages or both. The application may, for example, be done by drops, sprays, mists, coatings or mixtures thereof applied to the nasal and sinus passages.

“Vaginal application”, as used herein, means applied into or through the vagina so as to contact vaginal mucosa. The application may contact the urethra, cervix, formix, uterus or other area surrounding the vagina. The application may, for example, be done by drops, sprays, mists, coatings, lubricants or mixtures thereof applied to the vagina or surrounding tissue.

As used herein, the term “kit” refers to any delivery system for delivering materials. In the context of immunogenic agents (e.g., compositions comprising a nanoemulsion and an immunogen), such delivery systems include systems that allow for the storage, transport, or delivery of immunogenic agents and/or supporting materials (e.g., written instructions for using the materials, etc.) from one location to another. For example, kits include one or more enclosures (e.g., boxes) containing the relevant immunogenic agents (e.g., nanoemulsions) and/or supporting materials. As used herein, the term “fragmented kit” refers to delivery systems comprising two or more separate containers that each contain a subportion of the total kit components. The containers may be delivered to the intended recipient together or separately. For example, a first container may contain a composition comprising a nanoemulsion and an immunogen for a particular use, while a second container contains a second agent (e.g., an antibiotic or spray applicator). Indeed, any delivery system comprising two or more separate containers that each contains a subportion of the total kit components are included in the term “fragmented kit.” In contrast, a “combined kit” refers to a delivery system containing all of the components of an immunogenic agent needed for a particular use in a single container (e.g., in a single box housing each of the desired components). The term “kit” includes both fragmented and combined kits.

### DETAILED DESCRIPTION OF THE INVENTION

The present invention provides methods and compositions for the stimulation of immune responses. In particular, the present invention provides nanoemulsion compositions and methods of using the same for the induction of immune responses (e.g., immunologic cell death/apoptosis (e.g., for the induction of innate and/or adaptive immune responses)). Compositions and methods of the invention find use in, among other things, clinical (e.g. therapeutic and preventative medicine (e.g., for infectious disease, cancer, autoimmunity, and/or tissue injury (e.g., via alteration of host immune responses))) and research applications.

Thus, in some embodiments, the invention provides nanoemulsion compositions and use of the same for the stimulation of immune responses (e.g., immunologic apoptosis (e.g., inducing innate and/or adaptive immune response)). In some embodiments, the invention provides methods of using nanoemulsion compositions for inducing immunologic cell death (e.g., in the context of treating and/or preventing infectious disease and/or cancer). In some embodiments, the invention provides nanoemulsion adjuvant compositions that stimulate and/or elicit immune responses (e.g., innate immune responses and/or adaptive/acquired immune responses) when administered to a subject (e.g., a human subject (e.g., for generating pathogen-specific immune responses (e.g., to therapeutically or prophylactically protect a subject (e.g., from disease (e.g., cancer) or infection (e.g., caused by a pathogen))))). In some embodiments, the invention provides nanoemulsion adjuvant compositions comprising one or a plurality of immunogens (e.g., pathogen components, inactivated pathogens, cancer epitope and/or antigen). The invention is not limited to any particular nanoemulsion or immunogen. Exemplary nanoemulsion compositions (e.g., vaccine compositions) and methods of using the same (e.g., to induce immunogenic apoptosis and/or innate or adaptive immune responses) are described herein.

The respiratory mucosa is a main portal of entry for many human pathogens (e.g., including, but not limited to, influenza, adenovirus, coronavirus, rhinovirus, respiratory syncytial virus, Mycobacteria tuberculosis, Streptococcus pneumonia, etc.). The nasal cavity is the first anatomical interface between airborne microorganisms and the airway mucosa (See, e.g., Ramanathan, M., Jr. and Lane, A. P, Otolaryngol Head Neck Surg 2007. 136: 348-356). To date, conventional understanding of antigen sampling across the nasal mucosa and the role that the various nasal cells and tissues (e.g., epithelial cells, M-cells, antigen presenting cells (APCs), lymphocytes, connective tissue, etc.) play in immunoregulation has remained enigmatic. Early events in nasal pathogen invasion and the penetration of microbes beyond the nasal sub-mucosa are believed to be important for the induction of innate and adaptive immune responses (See, e.g., Isaacson et al., Curr Top Microbiol Immunol 2008. 325: 85-100). However, challenges to effective mucosal vaccination have included difficulties in generating effective mucosal immune responses (e.g., mucosal immunity), and the lack of safe, effective mucosal adjuvants and delivery systems. Accordingly, experiments were conducted during development of embodiments of the invention in order to identify and characterize compositions and methods for inducing (e.g., that are involved in) antigen sampling, processing and distribution (e.g., in order to identify means to induce and/or enhance antigen sampling, processing and/or distribution (e.g., for the generation of innate and/or adaptive immune responses (e.g., that provide new and/or superior immunization strategies and/or compositions useful for the same))).

Transport of antigens across the nasal epithelial barrier is a preliminary step in the induction of a mucosal immune response and antigen sampling. This process is influenced by a number of factors. The nasal mucosa comprises epithelium coated with mucus above a serous pericililiary layer (See, e.g., Ooi et al., Am J Rhinol 2008. 22: 13-19). The epithelial, mucus and anionic glycocalyceal layers serve as physical barriers to block invasion of bacteria, viruses, fungi or toxins through a variety of mechanisms (See, e.g., Pickles, Proc Am Thorac Soc 2004. 1: 302-308). Entry of antigens or microbial pathogens into mucosal epithelium is dependent on molecular interaction between the surface of the foreign material and host cells receptors. These receptors include glycoproteins, proteoglycans and glycolipids, and complex transmembrane protein structures (See, e.g., Bomsel and Alfsen, Nat Rev Mol Cell Biol 2003. 4: 57-68; Joenvarra et al., J Allergy Clin Immunol 2009. 124: 135-142 e131-121). A main function of innate mucosal cells, such as macrophages, dendritic cells (DCs) and respiratory epithelial cells is to identify dangerous microorganisms, through the recognition of specific pathogen-associated molecular patterns (PAMP). This takes place through the activation of a variety of receptor structures including Toll-like receptors (TLRs), NOD-like receptors, retinoic acid (RA)-inducible gene I-like helicases, and C-type lectins (See, e.g., Ichinohe et al., J Exp Med 2009. 206: 79-87). Translocation of particulate antigens, intact bacteria or viruses from the upper airways to APCs, is believed to occur in non-ciliated microfold cells (M-cells) (See, e.g., Kraehenbuhl and Neutra, Annu Rev Cell Dev Biol 2000. 16: 301-332) that are located in specialized tissues such as the Waldeyer's ring in humans and nasal-associated lymphoid tissue (NALT) in rodents (See, e.g., Spit et al., Cell Tissue Res 1989. 255: 193-198). Also, while non-soluble antigen uptake does not occur in sinonasal ciliated epithelial cells (See, e.g., Giannasca et al., Infect Immun 1997. 65: 4288-4298), TLR activated DCs (e.g., CD11c+ CX3CR1+ cells) residing in the basolateral side of gut mucosal tissues extend processes across the tight junctions between epithelial cells and have been documented to be capable of capturing pathogens (See, e.g., Chieppa et al., J Exp Med 2006. 203: 2841-2852). It is unclear whether this process occurs in nasal mucosa, although direct luminal sampling by DC is likely in areas of nasal stratified squamous epithelia where no directional trans-cytosis is thought to occur (See, e.g., Neutra et al., Annu Rev. Immunol. 1996. 14: 275-300).

The balance between inflammation/immunity and tolerance in the nasal mucosa is related to early recognition and entry events in the mucosa. Innate immune recognition has been shown to be important for the development of antigen-specific T-helper cell type 1 and 2 (Th1 and Th2) responses in non-respiratory epithelium (See, e.g., Sato and Iwasaki, Proc Natl Acad Sci USA 2004. 101: 16274-16279; Liu et al., Annu Rev Immunol 2007. 25: 193-219; Rimoldi et al., Nat Immunol 2005. 6: 507-514; Zaph et al., Nature 2007. 446: 552-556; Minns et al., J Immunol 2006. 176: 7589-759). The cytokine profile in mucosal epithelium associated with activation of MHC class I-restricted cytotoxic T lymphocytes (CTL) has been characterized to involve the epithelial cell or APC production of IL-1, IL-12, IL-18, GM-CSF and IFN-γ (See, e.g., Staats et al., J Immunol 2001. 167: 5386-5394). The involvement of stromal-cell mediated innate immunity in nasal mucosa has heretofore remained unknown.

Enterotoxin-based adjuvants, such as cholera toxin (CT) and E. coli heat-labile toxin (LT), which produce Th2-biased immune responses (See, e.g., Marinaro et al., J. Immunol. 1995. 155: 4621-4629) to co-administered antigens have been documented to effect antigen trafficking when administered intranasally (See, e.g., Van Ginkle et al., Infect Immun 2005. 73: 6892-6902). However, while CT and LT enhance antigen sampling, they also cause toxicity and significant inflammation via ADP-ribosyltransferase activity that permeabilizes the nasal epithelium to allow entry of vaccine proteins into sub-epithelial and olfactory tissues. This latter activity has prevented clinical use due to the associated toxicity and the potential for brain inflammation (See, e.g., Mutsch et al., N Engl J Med 2004. 350: 896-903; Sjoblom-Hallen et al., Mucosal Immunol 2010. 3: 374-386).

Experimental adjuvant formulations containing TLRs agonists stimulate innate immune responses and promote protection against pathogen challenge (See, e.g., Belyakov et al., Blood 2006. 107: 3258-3264; Sui et al., Proc Natl Acad Sci USA 2010. 107: 9843-9848). Adjuvant systems utilizing TLR signaling have been approved for human use in both the United States and Europe. One such example is CERAVIX (GlaxoSmithKline) which is a combination of ALUM and MPL (A TLR 4 ligand). Additionally, several micro- and nano-polymeric vaccine delivery systems do not activate immune cells but protect antigens from proteases in the mucosa. These materials appear to enhance antigen uptake through trans-cytosis in non-ciliated epithelium or M-cells, but the induction of robust or protective immune response (e.g., immunity) with these materials alone has been poor, requiring therefore the addition of inflammatory compounds in order to generate effective immune responses (See, e.g., Borges et al., Pharm Res 2010. 27: 211-223).

Experiments were conducted during development of embodiments of the invention in order to characterize and understand the adjuvant activity of a novel soybean oil-in-water nanoemulsion (NE) (e.g., used as an adjuvant (e.g., nasal adjuvant)). When mixed with antigen, the nanoemulsion promoted robust mucosal immunity, high serum antibody titers and cellular immunity that comprises both Th1 and Th17 responses (See, e.g., Bielinska et al., AIDS Res Hum Retroviruses 2008. 24: 271-281; Bielinska et al., Crit Rev Immunol 2010. 30: 189-199; Makidon et al., PLoS ONE 2008. 3: e2954).

The activity (e.g., adjuvant activity) of NE described herein displayed distinctly different properties than conventional adjuvants in that NE failed to induce histological nasal inflammation and also failed to provoke epithelial disruption, both of which, as referenced above and documented herein, have heretofore been thought to be of critical importance in the induction of robust and effective immune responses (e.g., protective immune responses (e.g., immunity)) (See, e.g., Bielinska et al., AIDS Res Hum Retroviruses 2008. 24: 271-281; Makidon et al., PLoS ONE 2008. 3: e2954; Bielinska et al., Infect Immun 2007. 75: 4020-4029; Bielinska et al., Clin Vaccine Immunol 2008. 15: 348-358). Accordingly, experiments were conducted during development of embodiments of the invention in order to identify and characterize properties of NE and methods of using the same that have heretofore remained unknown. As described herein, in some preferred embodiments, the invention provides that NE compositions disclosed herein are used to induce immunologic apoptosis/cell death (e.g., that induce innate and/or adaptive immune responses (e.g., not achievable with conventional, toxin based-adjuvants (e.g., CT, LT, alum, etc.). In some embodiments, a NE composition of the invention is used to induce immunomodulatory activities (e.g., induction of cytokine expression and/or signaling profiles) that are different from immunomodulatory activities induced by toxin based adjuvants. In some embodiments, a NE composition of the invention is used to induce antigen trafficking activities (e.g., via ciliated epithelial cells) that are different from antigen trafficking activities induced by toxin based adjuvants (e.g., via classical antigen presenting cells (e.g., macrophages and/or dendritic cells)).

Generation of mucosal innate and adaptive immune responses (including Th1, Th17, high avidity CD8+ CTL, neutralizing IgG1 Abs and secretory IgA) at the site of mucosal entry may be important for effective protection against pathogens that lead to chronic infection (See, e.g., Belyakov et al., Blood 2006. 107: 3258-3264;Sui et al., Proc Natl Acad Sci USA 2010. 107: 9843-9848; Mascola et al., Nat Med 2000. 6: 207-210; Belyakov et al., J Immunol 2007. 178: 7211-7221). Accordingly, in some embodiments, the invention provides adjuvants (e.g., mucosal adjuvants (e.g., NE adjuvants) that improve the efficacy of mucosal vaccination (e.g., although an understanding of a mechanism is not necessary to practice the invention and while the invention is not limited to any particular mechanism of action, in some embodiments, the invention provides NE that participate in targeting viral antigens to mucosal CD103+ DC (e.g., that imprint mucosal-homing receptors on pathogen-specific T and B cells and the induction of Th1/Th17 responses, See, e.g., Examples 2-12)).

Experiments were conducted during development of embodiments of the invention in order to characterize and understand biological properties of NE described herein. For example, an enhanced understanding of the biological properties of NE adjuvant (e.g., mucosal adjuvant) as well as other, convention adjuvants (e.g., cholera toxin), contribute to the rationale design of new and/or improved NE adjuvant formulations and uses. Accordingly, as described herein, the invention provides NE adjuvants and methods of using the same that provoke and/or enhance active uptake of antigen (e.g., by non-traditional antigen presenting cells) when administered to a subject as well as that provoke and/or induce immunoregulatory cytokine production by ciliated nasal epithelial cells in the absence of inflammation (See, e.g., Examples 3-12). Accordingly, in some embodiments, the invention provides NE and methods of using the same to effect antigen internalization (e.g., in nasal mucosa (e.g., by ciliated nasal epithelial cells)), antigen uptake and trafficking to regional (e.g., draining) lymph nodes, and recruitment of lymphocytes to the site of immunization (e.g., nasal associated lymphoid tissue (NALT)) (See, e.g., Examples 3-10).

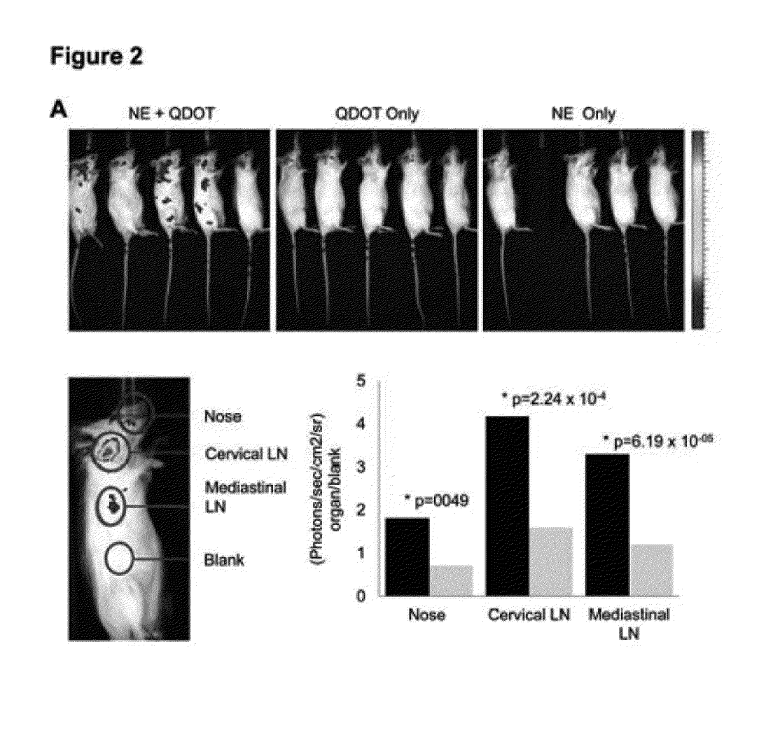

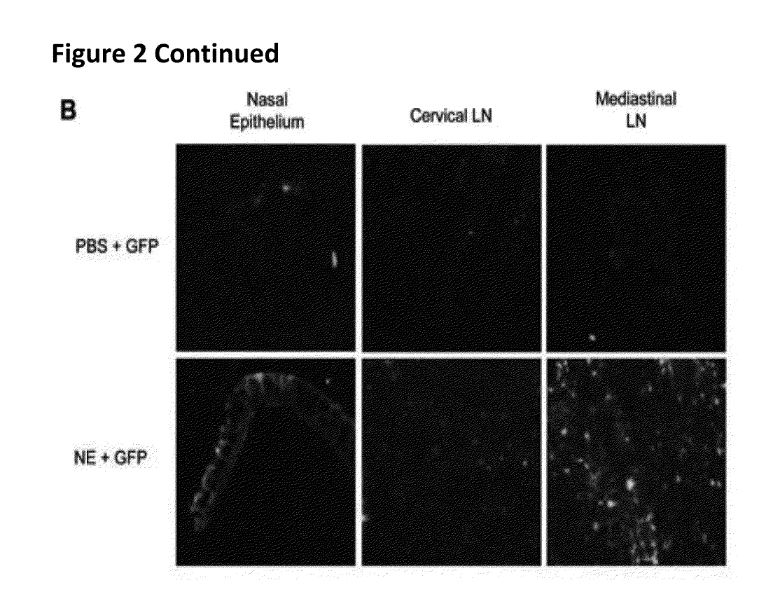

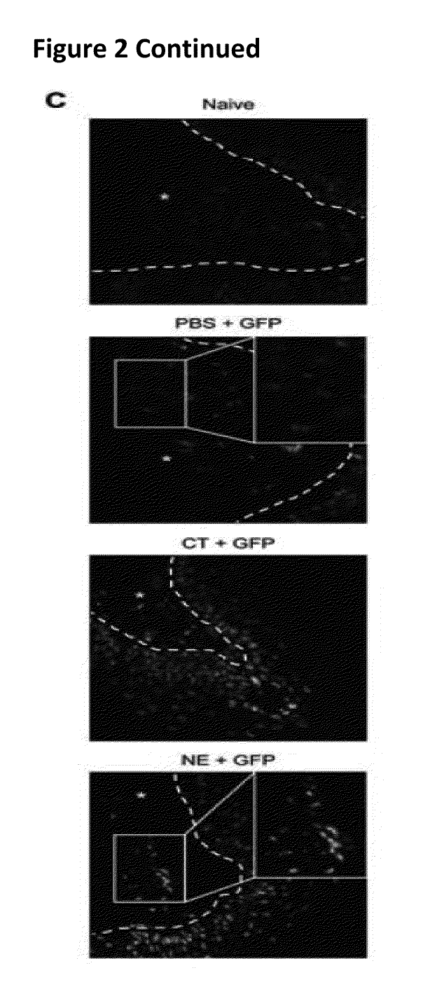

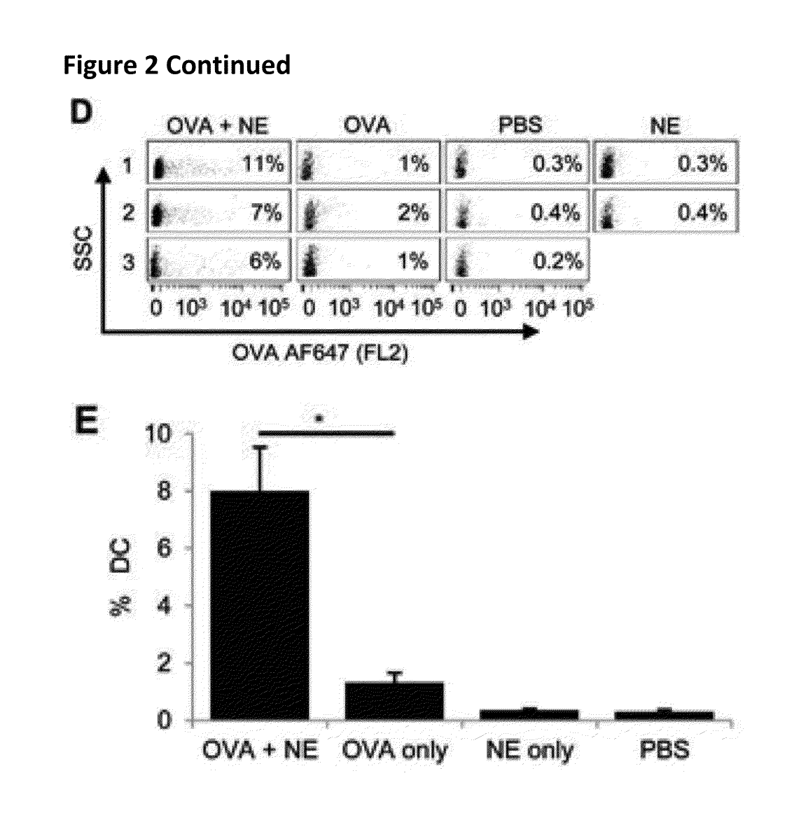

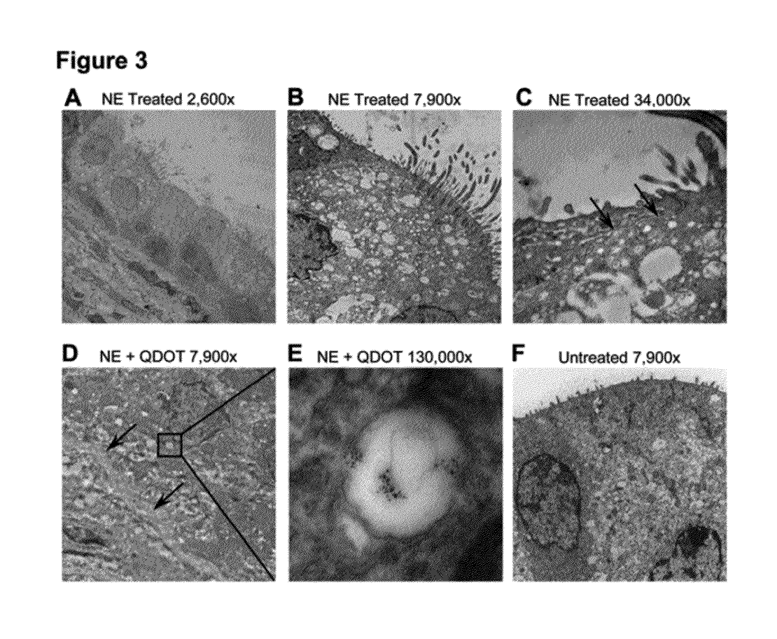

It has heretofore remained conventional wisdom that particulate antigen sampling did not occur in ciliated mucosal epithelial cells. However, experiments carried out during development of embodiments of the invention have characterized, for the first time, that NE enhanced antigen uptake in NALT, and that the antigen uptake involved ciliated epithelial cells (See, e.g., FIG. 2-3). The presence of emulsion droplets containing QDOTs in ciliated epithelial cells (See, e.g., FIGS. 3D and 3E) indicates that the ciliated nasal epithelial cells internalize intact complexes of NE and QDOTs (e.g., although an understanding of a mechanism is not necessary to practice the invention and while the invention is not limited to any particular mechanism of action, in some embodiments, the invention provides that antigen is internalized by ciliated nasal epithelial cells via a macropinocytic-like process that does not disrupt tight junctions or cell membranes). Thus, in some embodiments, the invention provides compositions (e.g., comprising NE) and methods of inducing antigen internalization via non-traditional antigen presenting cells (e.g., via ciliated epithelial cells) present within nasal associated lymphoid tissue (NALT) and subsequent innate and immune responses (e.g., those described herein).

Experiments conducted during development of embodiments of the invention provide that nasal mucosa NE antigen uptake did not require M-cell or direct DC luminal sampling. This unique route of facilitated antigen uptake in nasal tissues appeared to be trans-cellular as opposed to para-cellular as evidenced by the finding that tight junctions remained intact despite the presence of NE-associated vesicles (See, e.g., FIG. 3C). Further, permeablization or disruptive changes in the epithelial layer were absent (in contrast to significant inflammation and epithelial layer disruption observed with conventional adjuvants (e.g., CT)) as documented by the EM images, the lack of histological inflammation and the absence of clinical side effects. Thus, the invention provides, in some embodiments, NE and methods of using the same to induce unique immune responses (e.g., immunologic apoptosis (e.g., that induce innate and adaptive immune responses)) via loading antigens into epithelia cells. As such, the invention provides new pathways for inducing innate and adaptive immune responses (e.g., both local and systemic immune responses) via mucosal immunization (e.g., that leads to DC migration from mucosa to systemic lymphatic tissues).

Although an understanding of a mechanism is not necessary to practice the invention and while the invention is not limited to any particular mechanism of action, in some embodiments, the invention provides that the ability of NE to promote and/or induce trans-cellular antigen uptake is related to “lipidizing” proteins (e.g., within a NE particle) thereby enhancing trans-cellular absorption (e.g., in respiratory epithelial barriers). In some embodiments, mixing antigen with nanoemulsion traps antigen in the oil phase thereby inducing transcellular migration across the apical membrane, through the cell cytoplasm, and across the basolateral membrane. This route of permeation for hydrophobic compounds across the mucosa is very efficient given that the surface area of the transcellular approach is much larger by a factor of 9999 to 1 than the surface area of the paracellular route (tight junctions) (See, e.g., Pappenheimer et al., J Membr Biol 1987. 100: 123-136; Madara and Pappenheimer, J Membr Biol 1987. 100: 149-164).

The data and information generated during development of embodiments of the invention provide a new and enhanced understanding of mucosal antigen sampling. Accordingly, the invention provides new and useful methods for inducing innate and/or adaptive immune responses (e.g., via immunologic apoptosis (e.g., for the prevention and/or treatment of infections and/or disease transmitted through mucosal surfaces)). For example, in some embodiments, the invention provides methods of inducing Th1/Th17 type immune responses in a subject via mucosal administration of composition comprising NE (e.g., with an immunogen and/or antigen) to the subject. Although an understanding of a mechanism is not necessary to practice the invention and while the invention is not limited to any particular mechanism of action, in some embodiments, because the emulsion droplets are approximately the same size as viruses, are highly surface active and readily endocytosed by epithelial cells, the invention provides that lipid droplets penetrating the nasal mucosa induce Th1/Th17 type immune responses that mimic how mammalian immune systems have evolved to deal with lipid-covered respiratory viruses.

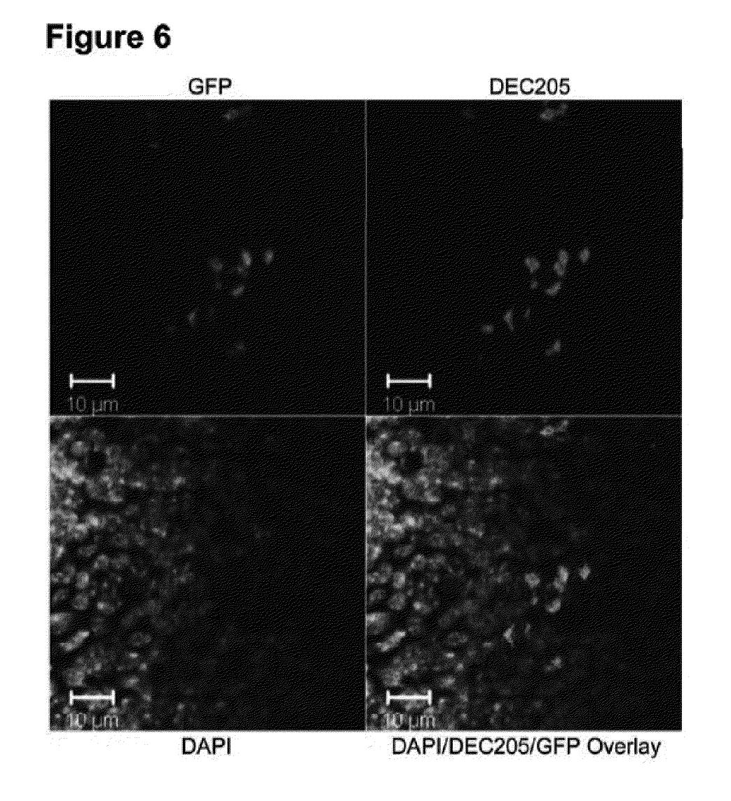

In some embodiments, the invention provides that antigen loading of DC after intranasal vaccination with NE occurs in the epithelia since the NE-antigen complex does not appear to penetrate below the epithelium (See, e.g., FIG. 2B). Following epithelium cell uploading of NE-antigen complex, DEC205⁺ cells appeared to locally sample antigen in the epithelia and then migrate to the systemic lymphatic circulation (See, e.g., FIG. 6). Thus, the invention provides, in some embodiments, the ability to target antigen loading to DC occurs via epithelial cell antigen loading (e.g., non-traditional, non-professional antigen presenting cell loading). In further embodiments, DC antigen loading via epithelial cell antigen loading plays an important role in the migration of antigen loaded DCs to local, draining lymph nodes and/or to the recruitment of lymphocytes to the local, draining lymph nodes. Although an understanding of a mechanism is not necessary to practice the invention and while the invention is not limited to any particular mechanism of action, in some embodiments, the invention provides that chemical and/or biological properties of NE (e.g., including, but not limited to, mucoadhesion properties, antigen uptake induction, toxicity or lack thereof, and/or induction of cytokine induction/secretion) are involved in the induction of innate and adaptive immune responses that occur post administration of NE to a subject.

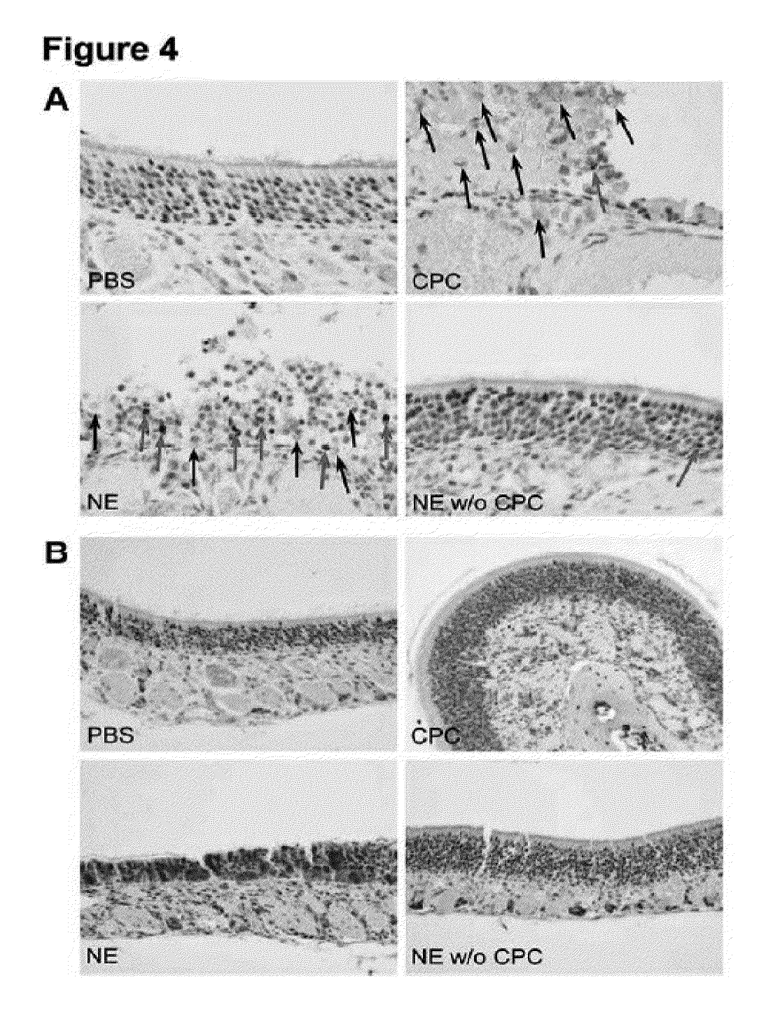

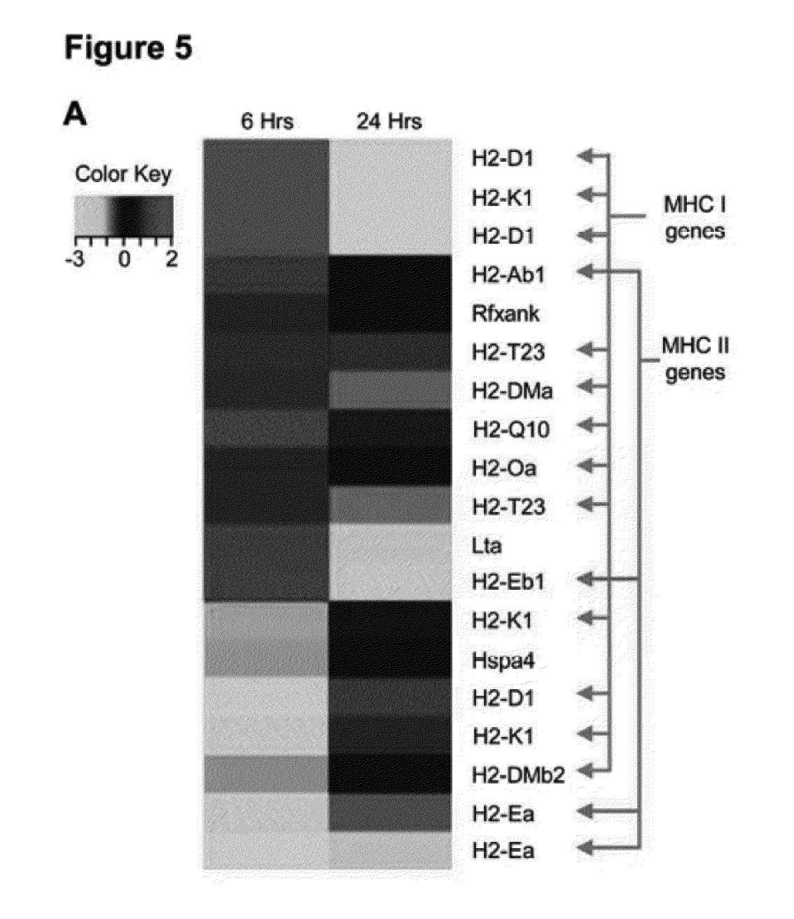

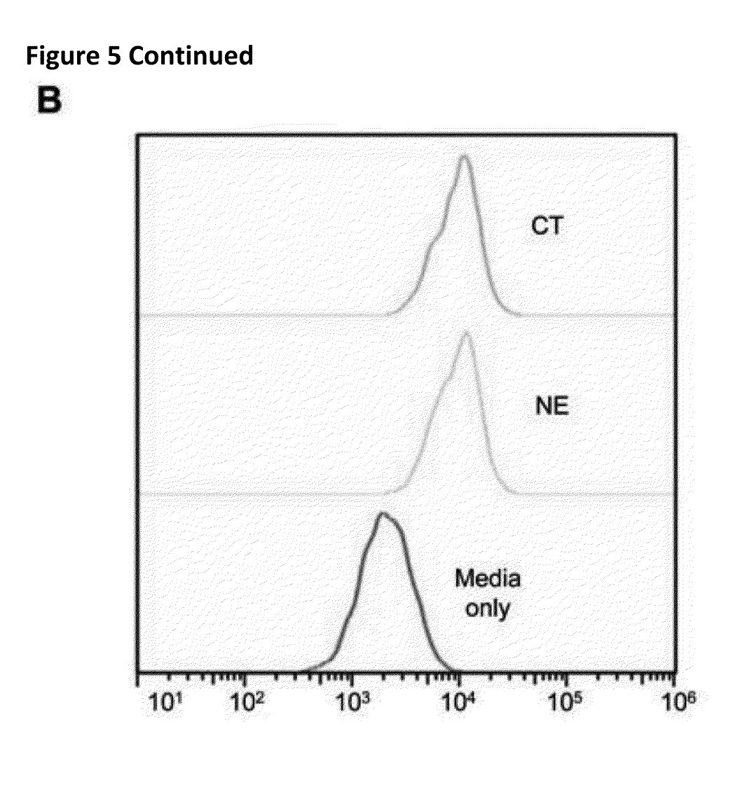

As disclosed herein, the invention provides that NE-antigen containing ciliated epithelial cells initiate immune responses in the nares. For example, it is shown that NE and antigen-loaded epithelial cells undergo immunogenic apoptosis and necrosis (See, e.g., FIG. 4). Although an understanding of a mechanism is not necessary to practice the invention and while the invention is not limited to any particular mechanism of action, in some embodiments, the invention provides that DC take up and/or engulf antigen-loaded dead cells. In some embodiments, host DNA released from dying cells acts as a damage-associated molecular pattern (DAMP) that mediates NE adjuvant activity. In some embodiments, local and/or systemic immune responses are a result of direct interaction between NE and antigen-loaded ciliated epithelial cells and antigen-specific CD4⁺ and CD8⁺ cells in sinonasal epithelium. For example, the invention provides that NE stimulates accessory antigen processing and presentation activities by MHC class I and II in epithelial cells (See, e.g., FIG. 5). In other embodiments, the invention provides that epithelial cells secrete biologically active exosomes capable of uptake in DC or presenting antigenic peptide in the context of MHC class I or class II to naïve T cells (See, e.g., Kesimer et al., Faseb J 2009. 23: 1858-1868; Van Niel et al., Gut 2003. 52: 1690-1697).

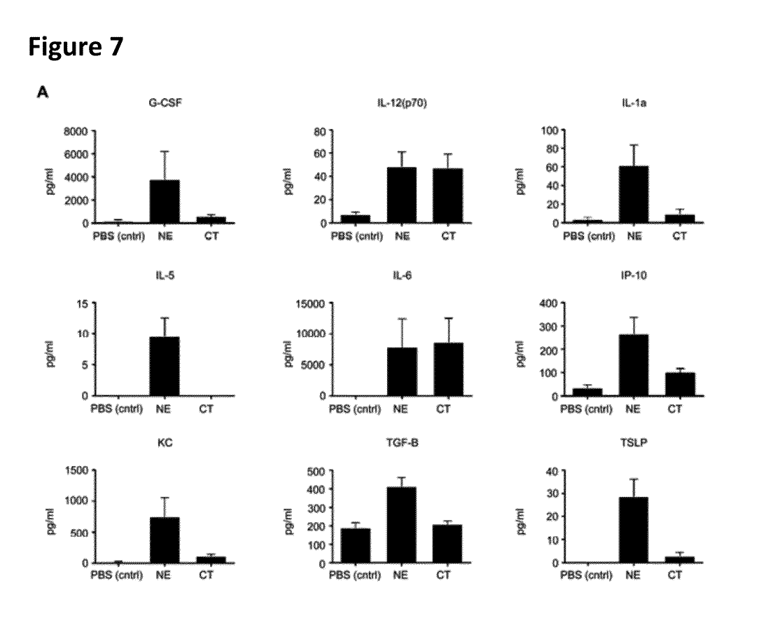

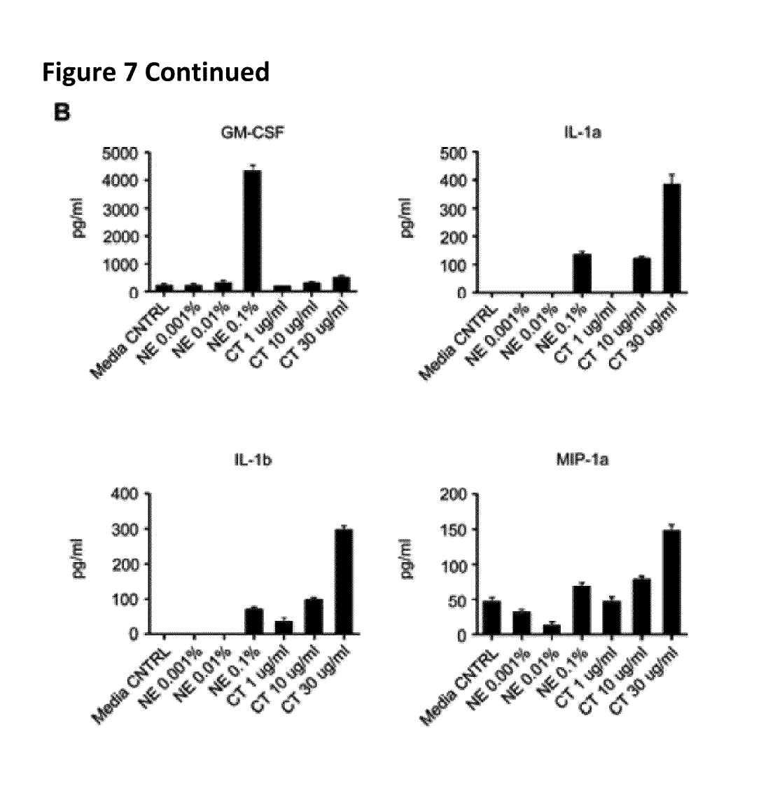

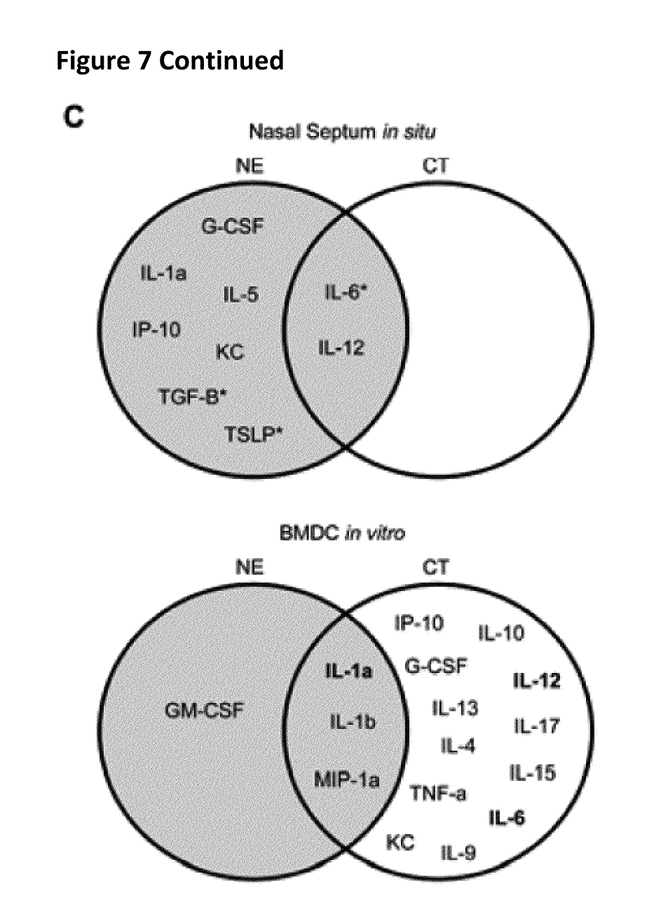

The invention provides that NE adjuvant mediated immune responses are quite distinct from immune responses induced by conventional adjuvants (e.g., CT) (See, e.g., Example 11 and FIG. 7). For example, the invention provides NE and use of the same to induce specific innate cytokine response profiles from numerous cell types (e.g., including non-traditional APCs). Thus, in some embodiments, the invention provides use of a NE to induce a cytokine profile (See, e.g., FIG. 7) in a subject that is distinct from a cytokine profile induced by a conventional adjuvant (e.g., CT) (e.g., in a method of preventing and/or treating infection and/or disease).

The vast majority of mucosal adjuvants cause local inflammation that attracts and activates antigen-presenting cells through cytokines, chemokines and multiple signaling pathways such as MyD88 (See, e.g., Eisenbarth et al., Nature 2008. 453: 1122-1126; van Duin et al., Trends Immunol 2006. 27: 49-55). This microenvironment facilitates local antigen sampling by DC and enhances presentation to the immune system after CT administration. However, the invention provides, in some preferred embodiments, NE and use of the same to induces unique and balanced production of cytokines in epithelium, in the absence of acute nor chronic histological inflammatory changes, and in the absence of antigen redirection to olfactory tissues (e.g., via NE administration to a subject). Based upon microarray results together with cytokine analysis, the invention provides compositions and methods for unique mucosal alterations in signaling pathways (e.g., that are induced by NE via influencing/altering antigen uptake, innate/adaptive immune responses and in the absence of inflammation). In further preferred embodiments, the invention provides compositions and methods for induction of cytokine/chemokine signaling that occurs predominately through activated epithelial cells and from mucosal DC activated by epithelial cells. Thus, in some embodiments, the invention provides compositions comprising NE as a nasopharyngeal vaccine adjuvant (e.g., due to the absence of retrograde transport to olfactory tissues in the brain).

The invention further provides compositions and methods for inducing unique and balanced cytokine profiles in a subject comprising adaptive Th17 immune responses. In the intestinal mucosa Th17 cells are the main source of IL-17, whereas in the respiratory mucosa γδT cells, NKT, NK, RORγt, and NKp46+ cells are the main producers of IL-17. Effector anti-bacterial and anti-fungi functions of IL-17 are attributed to the interaction with IL-17R expressed on fibroblasts and epithelial cells to induce MCP-2, G-CSF and CXC chemokines IL-22 is another important cytokine produced by Th17 for inducing the secretion of antimicrobial peptides and β-defensin-2 by epithelial cells and for contributing to barrier function and tissue repair (See, e.g., Lochner et al. J Exp Med 2008. 205: 1381-1393). In some embodiments, the invention provides that NE described herein is used to induce production of IL-17 (e.g., via mucosal administration of NE).

Experiments conducted during development of embodiments of the invention determined that IL-6 is a major cytokine associated with NE adjuvant effect (See, e.g., FIG. 7). Beyond its role in the Th17 pathway, IL-6 has important roles for the generation of immunity and antibody production following mucosal vaccination (See, e.g., Bettelli et al., Curr Opin Immunol 2007. 19: 652-657; Awasthi et al., Int Immunol 2009. 21: 489-498). In some embodiments, the invention provides that induction of IL-6 by NE provides muco-protective down-regulation of TNF and IL-1 and enhances trans-cellular passage of microbes through epithelial barrier (See, e.g., Prins et al., Am J Surg 2002. 183: 372-383; Kida et al., Am J Physiol Lung Cell Mol Physiol 2005. 288: L342-349). In some embodiments, the invention provides safe and effective mucosal adjuvants and delivery systems.

Accordingly, the invention provides compositions and methods for the stimulation of immune responses. In some embodiments, the invention provides methods of therapeutically and/or prophylactically treating a condition that benefits from the induction of immunogenic apoptosis comprising administering to a subject (e.g., mammalian subject) in need thereof an effective amount of a composition comprising NE disclosed herein (e.g., an immunogenic composition). The invention is not limited by the type of condition treated. In some embodiments, the condition is infection (e.g., bacterial, viral, fungal, yeast, etc.). In some embodiments, the condition is a disease (e.g., cancer). In some embodiments, the invention provides use of a NE disclosed herein for inducing signaling pathways that activate immunogenic apoptosis (e.g., to induce innate and/or adaptive immune responses). In some embodiments, use of a NE activate apoptotic cells that possess endogenous adjuvant properties. In some embodiments, use of NE induce the exposure of heat shock proteins and/or other chaperone proteins on cellular surfaces (e.g., ciliated epithelial cells). In some embodiments, apoptotic cells induced by a NE of the invention emit and/or secrete signals that attract antigen presenting cells (e.g., macrophages, dendritic cells, B cells (e.g., that in turn stimulate NE specific immune responses (e.g., those disclosed herein))). In some embodiments, use of NE induces release of or surface expression of damage-associated molecular patterns (DAMPs). The invention is not limited by the type of DAMP released or expressed. For example, the DAMP may be a nucleotide product (e.g., uric acid), a nuclear and/or DNA binding protein (e.g., high-mobility group box 1 protein (HMGB1) 47, etc. In some embodiments, use of NE induces caspase activation. In some embodiments, use of NE is used to traffic antigen to local lymph nodes (e.g., away from the spleen).

Immune response to cell death depends on the nature in which cells die, where they die, how they die, which cell engulfs them and when (or if) an associated antigen has been or will be recognized. Variations in these factors can have consequences that range from effective anti-pathogen or anti-tumor responses to autoimmune pathology (See, e.g., Green et al., Reviews Immunol., 2009, 9, 353-363). Classically described apoptosis, also referred to as “non-immunogenic apoptosis” is associated with Caspase3/7 expression and is one pathway involved in anti-pathogen and anti-tumor activity. Non-immunogenic apoptosis is understood as referring to apoptotic events in which apoptotic cells are tolerogenic or non-immunogenic.

However, an alternative “immunogenic apoptotic” pathway involving pre-apoptotic activation of caspase-8 and surface expression of calreticulin has also been described (See, e.g., Green et al., Reviews Immunol., 2009, 9, 353-363; Panaretakis et al., EMBO, 2009, 28, 578-590). Immunogenic apoptosis has many of the major hallmarks of non-immunogenic apoptosis except that it can activate (rather than suppress) the immune system by emitting distinct “immunogenic signals” comprising damage-associated molecular patterns (DAMPs). Thus, cells undergoing immunogenic apoptosis have acquired the ability to communicate their antigenic memory to the immune system thereby leading to potent anti-pathogen or anti-tumor immunity (See, e.g., Galluzzi et al., EMBO J. 2012; 31:1062-79; Garg et al. Biochim Biophys Acta 2010; 1805:53-71; Garg et al., EMBO J. 2012; 31:1055-7; Obeid et al., Nat Med. 2007; 13:54-61; Garg et al., Oncoimunology 2012; 1(5): 786-788)). Calreticulin serves as one major “eat me” signal for phagocytes to clear the dying and dead cells (See, e.g., Clarke et al., Nat Biotechnol 25: 192-193).

Accordingly, experiments were conducted during development of embodiments of the invention in order to determine whether nanoemulsion adjuvants promoted immunogenic apoptosis via activation of caspase 8. It was discovered that human nasal septum cells administerd/exposed to nanoemulsion displayed a dose-dependent increase in expression of caspase-8 (See Example 13). However, the dose-dependent activation of caspase 8 terminated at higher NE concentrations (e.g., >0.045%), whereby expression of active caspase 8 was inhibited (e.g., indicating that these cells die due to necrosis and/or lysis rather than apoptosis (e.g., due to cytotoxic amount of NE)). Accordingly, although an understanding of a mechanism of action is not needed to practice the present invention, and while the present invention is not limited to any particular mechanism of action, in some embodiments, the presence of dead or dying cells (e.g., caspase-8 induced apoptotic cells alone or in combination with necrotic cells) generates danger signals that stimulate migration of antigen presenting cells (APCs), facilitates antigen uptake, and induces the maturation of dendritic cells (See, e.g., Albert et al., Nature 1998; 392:86-9; Sauter et al. J Exp Med 2000; 191:423-34), whereby dendritic cells subsequently load antigens on MHC class I and II and trigger downstream antigen-specific immune responses (See e.g., Guermonprez et al., Annu Rev Immunol 2002; 20:621-67; Iyoda et al., J Exp Med 2002; 195:1289-302.))

Thus, the invention provides, in some embodiments, a method of stimulating immunogenic apoptosis in a subject in need thereof comprising administering (e.g., to a mucosal surface) to the subject an effective amount of a composition comprising a nanoemulsion to induce immunogenic apoptosis. In some embodiments, inducing immunogenic apoptosis in a subject comprises the activation, induction, stimulation and/or augmentation of caspase 8 activity. In some embodiments, activation, induction, stimulation and/or augmentation of caspase 8 activity results in calreticulin expression in the subject (e.g., in the nasal mucosa of the subject). Calreticulin is a multicompartmental protein that regulates a wide array of cellular responses important in physiological and pathological processes, such as wound healing, the immune response, fibrosis, and cancer. Thus, the invention is not limited by the type of subject that benefits from the induction of immunogenic apoptosis. Indeed, a variety of subjects will benefit from the induction of immunogenic apoptosis via administration of an effective amount of a nanoemulsion described herein including, but not limited to, a subject with cancer, a subject with a wound, and a subject with fibrosis (See, e.g., Gold et al. FASEB 2010; 24(3), 665-683). In some embodiments, the invention provides use of a composition comprising a nanoemulsion (e.g., disclosed herein) in the manufacture of a medicament for the induction of immunogenic apoptosis and/or induction of caspase 8 activity (e.g., for the treatment or prevention of a disease or condition (e.g., cancer, fibrosis and/or a wound).

The invention provides nanoemulsion adjuvants compositions and methods of using the same (e.g., individually, or together with one or more antigens/immunogens or components thereof (e.g., recombinant proteins) to induce an immune response in a subject (e.g., to prime, enable and/or enhance an immune response (e.g., against infection or disease in a subject)). Compositions and methods of the invention find use in, among other things, clinical (e.g. therapeutic and preventative medicine (e.g., vaccination)) and research applications. In some embodiments, a nanoemulsion adjuvant of the invention is utilized by itself, or together with another adjuvant (e.g., another nanoemulsion adjuvant and/or non-nanoemulsion adjuvant) in the absence of an antigen/immunogen present in the emulsion to stimulate an immune response (e.g., innate immune response and/or adaptive immune response) in a host subject. In some embodiments, one or a plurality of pathogens are mixed with a nanoemulsion adjuvant prior to administration (e.g., for a time period sufficient to inactivate the one or plurality of pathogens). In some embodiments, one or a plurality of protein components (e.g., isolated and/or purified and/or recombinant protein) from one or a plurality of pathogens are mixed with the nanoemulsion.

Although an understanding of the mechanism is not necessary to practice the present invention and the present invention is not limited to any particular mechanism of action, in some embodiments, nanoemulsion adjuvants penetrate mucosa to which it is administered (e.g., through pores) and carry immunogens to submucosal locations (e.g., harboring dendritic cells (e.g., thereby initiating and/or stimulating an immune response)). In some embodiments, nanoemulsion adjuvants of the invention preserve and/or stabilize antigenic epitopes (e.g., recognizable by a subject's immune system), stabilizing their hydrophobic and/or hydrophilic components in the oil and water interface of the emulsion (e.g., thereby providing one or more immunogens (e.g., stabilized antigens) against which a subject can mount an immune response). In some embodiments, a nanoemulsion adjuvant of the invention (e.g., comprising one or more protein and/or cellular antigens) creates an environment in which a protein or cellular antigen is maintained for a longer period of time in a subject (e.g., thereby providing enhanced opportunity for the protein or cellular antigen to be recognized and responded to by a host immune system).

Although an understanding of the mechanism is not necessary to practice the present invention and the present invention is not limited to any particular mechanism of action, in some embodiments, because the nasal cavity of a host subject comprises an overall negative charge, nanoemulsion adjuvants comprising cationic properties exhibit enhanced muco-adhesive properties compared to other materials lacking cationic properties. In some embodiments, combining a nanoemulsion adjuvant and one or a plurality of immunogenic proteins stabilizes the immunogens and provides a proper immunogenic material for generation of an immune response.

Both cellular and humoral immunity play a role in protection against multiple pathogens and both can be induced with the NE adjuvant formulations of the present invention. Thus, in some embodiments, administration (e.g., mucosal administration) of a nanoemulsion adjuvant of the present invention primes, enables and/or enhances induction of both humoral (e.g., development of specific antibodies) and cellular (e.g., cytotoxic T lymphocyte) immune responses (e.g., against a pathogen). In some embodiments, a nanoemulsion adjuvant composition of the present invention is used in a vaccine (e.g., as an immunostimulatory adjuvant (e.g., that elicits and/or enhances immune responses (e.g., innate and or adaptive immune responses) in a host administered the nanoemulsion adjuvant).

Furthermore, in some embodiments, a composition of the present invention (e.g., a composition comprising a NE adjuvant) induces (e.g., when administered to a subject) both systemic and mucosal immune responses (e.g., generates systemic and or mucosal immunity). Thus, in some embodiments, administration of a composition of the present invention to a subject results in protection against an exposure (e.g., a lethal mucosal exposure) to one or a plurality of pathogens (e.g., one or a plurality of viruses and/or bacteria). Although an understanding of the mechanism is not necessary to practice the present invention and the present invention is not limited to any particular mechanism of action, mucosal administration provides protection against pathogen infection (e.g., that initiates at a mucosal surface).

In some embodiments, the present invention provides nanoemulsion adjuvant compositions that replace the use of other adjuvants (e.g., adjuvants that cause inflammation, morbidity, and/or adverse side reactions in a host administered the composition). For example, in some embodiments, a nanoemulsion adjuvant of the invention is utilized in an immunogenic composition (e.g., a vaccine) in place of a Th1-type adjuvant. In some embodiments, a nanoemulsion adjuvant of the invention is utilized in an immunogenic composition (e.g., a vaccine) in place of a Th2-type adjuvant. In some embodiments, a nanoemulsion adjuvant of the invention provides, when administered to a host subject, an immune response (e.g., an innate, cell mediated, adaptive and/or acquired immune response) that is similar to, the same as, or greater than an immune response elicited by a conventional adjuvant compositions (e.g., cholera toxin, CpG oligonucleotide, alum, and/or other adjuvant described herein) without adverse and/or unwanted side-effects.

In some embodiments, the present invention provides compositions for inducing immune responses comprising a nanoemulsion adjuvant (e.g., independently and/or combined with one or more immunogens (e.g., inactivated pathogens or pathogen products)). A variety of nanoemulsion that find use in the present invention are described herein and elsewhere (e.g., nanoemulsions described in U.S. Pat. Apps. 20020045667 and 20040043041, and U.S. Pat. Nos. 6,015,832, 6,506,803, 6,635,676, and 6,559,189, each of which is incorporated herein by reference in its entirety for all purposes).

Nanoemulsion adjuvants (e.g., independently or combined with one or more immunogens (e.g., pathogens or pathogen products)) of the present invention may be combined in any suitable amount utilizing a variety of delivery methods. Any suitable pharmaceutical formulation may be utilized, including, but not limited to, those disclosed herein. Suitable formulations may be tested for immunogenicity using any suitable method. For example, in some embodiments, immunogenicity is investigated by quantitating both specific T-cell responses and antibody titer. Nanoemulsion compositions of the present invention may also be tested in animal models of infectious disease states. Suitable animal models, pathogens, and assays for immunogenicity include, but are not limited to, those described herein.

In some embodiments, the present invention provides compositions and methods for skewing and/or redirecting a host's immune response (e.g., away from Th2 type immune responses and toward Th1 type immune responses) to one or a plurality of immunogens/antigens. In some embodiments, skewing and/or redirecting a host's immune response (e.g., away from Th2 type immune responses and toward Th1 type immune responses) to one or a plurality of immunogens/antigens comprises providing one or more antigens (e.g., recombinant antigens, isolated and/or purified antigens, and/or killed whole pathogens) that are historically associated with generation of a Th2 type immune response when administered to a subject (e.g., RSV antigen, hepatitis B virus antigen, etc.), combining the one or more antigens with a nanoemulsion of the invention (e.g., W805EC), characterizing the properties of the nanoemulsion-antigen mixture (e.g., characterizing the zeta potential and/or surface charge of the composition), identifying a nanoemulsion-antigen mixture that displays properties (e.g., positive surface charge, zeta potential above 30 mV, stability, etc.), identified as sufficient to generate a desired immune response (e.g., cell mediated immune response (e.g., Th1 type immune response)) when administered to a subject, and administering the nanoemulsion-antigen mixture to a subject under conditions sufficient to induce the desired immune response.

In some embodiments, the present invention provides adjuvants that reduce the number of booster injections (e.g., of an antigen containing composition) required to achieve protection. In some embodiments, the present invention provides adjuvants that result in a higher proportion of recipients achieving seroconversion. In some embodiments, the present invention provides adjuvants that are useful for selectively skewing adaptive immunity toward Th1, Th2, or cytotoxic T cell responses (e.g., allowing effective immunization by distinct routes (e.g., such as via the skin or mucosa)). In some embodiments, the present invention provides adjuvants that elicit optimal responses in subjects in which most contemporary vaccination strategies are not optimally effective (e.g., in very young and/or very old populations). In some embodiments, the present invention provides adjuvants that provide efficacy and safety needed for vaccination regimens that involve different delivery routes and elicitation of distinct types of immunity. In some embodiments, the present invention provides adjuvants that stimulate antibody responses and have little toxicity and that can be utilized with a range of antigens for which they provide adjuvanticity and the types of immune responses they elicit. In some embodiments, the present invention provides adjuvants that meet global supply requirements (e.g., in response to a pathogenic (e.g., influenza) pandemic).

### Generation of Antibodies

An immunogenic composition comprising a nanoemulsion adjuvant (e.g., independently or together with an antigen) can be used to immunize a mammal, such as a mouse, rat, rabbit, guinea pig, monkey, or human, to produce polyclonal antibodies. If desired, an antigen can be conjugated to a carrier protein, such as bovine serum albumin, thyroglobulin, keyhole limpet hemocyanin or other carrier described herein. Depending on the host species, various additional adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, nanoemulsions described herein, keyhole limpet hemocyanin, and dinitrophenol). Among adjuvants used in humans, BCG (bacilli Calmette-Guerin) and Corynebacterium parvum are especially useful.

Monoclonal antibodies can be prepared using any technique which provides for the production of antibody molecules by continuous cell lines in culture. These techniques include, but are not limited to, the hybridoma technique, the human B cell hybridoma technique, and the EBV hybridoma technique (See, e.g., Kohler et al., Nature 256, 495 497, 1985; Kozbor et al., J. Immunol. Methods 81, 3142, 1985; Cote et al., Proc. Natl. Acad. Sci. 80, 2026 2030, 1983; Cole et al., Mol. Cell. Biol. 62, 109 120, 1984).

In addition, techniques developed for the production of “chimeric antibodies,” the splicing of mouse antibody genes to human antibody genes to obtain a molecule with appropriate antigen specificity and biological activity, can be used (See, e.g., Morrison et al., Proc. Natl. Acad. Sci. 81, 68516855, 1984; Neuberger et al., Nature 312, 604 608, 1984; Takeda et al., Nature 314, 452 454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (See, e.g., Burton, Proc. Natl. Acad. Sci. 88, 11120 23, 1991).

Single-chain antibodies also can be constructed using a DNA amplification method, such as PCR, using hybridoma cDNA as a template (See, e.g., Thirion et al., 1996, Eur. J. Cancer Prey. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol. 15, 159-63. Construction of bivalent, bispecific single-chain antibodies is taught, for example, in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (See, e.g., Verhaar et al., 1995, Int. J. Cancer 61, 497-501; Nicholls et al., 1993, J. Immunol. Meth. 165, 81-91).

Antibodies can be produced by inducing in vivo production in the lymphocyte population or by screening immunoglobulin libraries or panels of highly specific binding reagents as disclosed in the literature (See, e.g., Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared. Antibodies can be purified by methods well known in the art. For example, antibodies can be affinity purified by passage over a column to which the relevant antigen is bound. The bound antibodies can then be eluted from the column using a buffer with a high salt concentration.

### Nanoemulsions

The present invention is not limited by the type of nanoemulsion adjuvant utilized (e.g., for respiratory administration). Indeed, a variety of nanoemulsion adjuvants are contemplated to be useful in the present invention.

For example, in some embodiments, a nanoemulsion comprises (i) an aqueous phase; (ii) an oil phase; and at least one additional compound. In some embodiments of the present invention, these additional compounds are admixed into either the aqueous or oil phases of the composition. In other embodiments, these additional compounds are admixed into a composition of previously emulsified oil and aqueous phases. In certain of these embodiments, one or more additional compounds are admixed into an existing emulsion composition immediately prior to its use. In other embodiments, one or more additional compounds are admixed into an existing emulsion composition prior to the compositions immediate use.

Additional compounds suitable for use in a nanoemulsion of the present invention include, but are not limited to, one or more organic, and more particularly, organic phosphate based solvents, surfactants and detergents, cationic halogen containing compounds, germination enhancers, interaction enhancers, food additives (e.g., flavorings, sweeteners, bulking agents, and the like) and pharmaceutically acceptable compounds (e.g., carriers). Certain exemplary embodiments of the various compounds contemplated for use in the compositions of the present invention are presented below. Unless described otherwise, nanoemulsions are described in undiluted form.

Nanoemulsion adjuvant compositions of the present invention are not limited to any particular nanoemulsion. Any number of suitable nanoemulsion compositions may be utilized in the vaccine compositions of the present invention, including, but not limited to, those disclosed in Hamouda et al., J. Infect Dis., 180:1939 (1999); Hamouda and Baker, J. Appl. Microbiol., 89:397 (2000); and Donovan et al., Antivir. Chem. Chemother., 11:41 (2000). Preferred nanoemulsions of the present invention are those that are non-toxic to animals. In preferred embodiments, nanoemulsions utilized in the methods of the present invention are stable, and do not decompose even after long storage periods (e.g., one or more years). Additionally, preferred emulsions maintain stability even after exposure to high temperature and freezing. This is especially useful if they are to be applied in extreme conditions (e.g., extreme heat or cold).

Some embodiments of the present invention employ an oil phase containing ethanol. For example, in some embodiments, the emulsions of the present invention contain (i) an aqueous phase and (ii) an oil phase containing ethanol as the organic solvent and optionally a germination enhancer, and (iii) TYLOXAPOL as the surfactant (preferably 2-5%, more preferably 3%). This formulation is highly efficacious for inactivation of pathogens and is also non-irritating and non-toxic to mammalian subjects (e.g., and thus can be used for administration to a mucosal surface).

In some other embodiments, the emulsions of the present invention comprise a first emulsion emulsified within a second emulsion, wherein (a) the first emulsion comprises (i) an aqueous phase; and (ii) an oil phase comprising an oil and an organic solvent; and (iii) a surfactant; and (b) the second emulsion comprises (i) an aqueous phase; and (ii) an oil phase comprising an oil and a cationic containing compound; and (iii) a surfactant.

### Exemplary Formulations

The following description provides a number of exemplary emulsions including formulations for compositions BCTP and X₈W₆₀PC. BCTP comprises a water-in oil nanoemulsion, in which the oil phase was made from soybean oil, tri-n-butyl phosphate, and TRITON X-100 in 80% water. X₈W₆₀PC comprises a mixture of equal volumes of BCTP with W₈₀8P. W₈₀8P is a liposome-like compound made of glycerol monostearate, refined oy a sterols (e.g., GENEROL sterols), TWEEN 60, soybean oil, a cationic ion halogen-containing CPC and peppermint oil. The GENEROL family are a group of a polyethoxylated soya sterols (Henkel Corporation, Ambler, Pa.). Exemplary emulsion formulations useful in the present invention are provided in Table 1. These particular formulations may be found in U.S. Pat. Nos. 5,700,679 (NN); 5,618,840; 5,549,901 (W₈₀8P); and 5,547,677, each of which is hereby incorporated by reference in their entireties. Certain other emulsion formulations are presented U.S. patent application Ser. No. 10/669,865, hereby incorporated by reference in its entirety.

The X₈W₆₀PC emulsion is manufactured by first making the W₈₀8P emulsion and BCTP emulsions separately. A mixture of these two emulsions is then re-emulsified to produce a fresh emulsion composition termed X₈W₆₀PC. Methods of producing such emulsions are described in U.S. Pat. Nos. 5,103,497 and 4,895,452 (each of which is herein incorporated by reference in their entireties).

TABLE 1

| | | Water |
|---|---|---|
| | Oil Phase Formula | to Oil Phase Ratio (Vol/Vol) |
| BCTP | 1 vol. Tri(N-butyl)phosphate | 4:1 |
| | 1 vol. TRITON X-100 | |
| | 8 vol. Soybean oil | |
| NN | 86.5 g Glycerol monooleate | 3:1 |
| | 60.1 ml Nonoxynol-9 | |
| | 24.2 g GENEROL 122 | |
| | 3.27 g Cetylpyridinium chloride | |
| | 554 g Soybean oil | |
| W₈₀8P | 86.5 g Glycerol monooleate | 3.2:1 |
| | 21.2 g Polysorbate 60 | |
| | 24.2 g GENEROL 122 | |
| | 3.27 g Cetylpyddinium chloride | |
| | 4 ml Peppermint oil | |
| | 554 g Soybean oil | |
| SS | 86.5 g Glycerol monooleate | 3.2:1 |
| | 21.2 g Polysorbate 60 | (1% bismuth in water) |
| | 24.2 g GENEROL 122 | |
| | 3.27 g Cetylpyridinium chloride | |
| | 554 g Soybean oil | |

The compositions listed above are only exemplary and those of skill in the art will be able to alter the amounts of the components to arrive at a nanoemulsion composition suitable for the purposes of the present invention. Those skilled in the art will understand that the ratio of oil phase to water as well as the individual oil carrier, surfactant CPC and organic phosphate buffer, components of each composition may vary.

Although certain compositions comprising BCTP have a water to oil ratio of 4:1, it is understood that the BCTP may be formulated to have more or less of a water phase. For example, in some embodiments, there is 3, 4, 5, 6, 7, 8, 9, 10, or more parts of the water phase to each part of the oil phase. The same holds true for the W₈₀8P formulation. Similarly, the ratio of Tri (N-butyl) phosphate: TRITON X-100: soybean oil also may be varied.

Although Table 1 lists specific amounts of glycerol monooleate, polysorbate 60, GENEROL 122, cetylpyridinium chloride, and carrier oil for W₈₀8P, these are merely exemplary. An emulsion that has the properties of W₈₀8P may be formulated that has different concentrations of each of these components or indeed different components that will fulfill the same function. For example, the emulsion may have between about 80 to about 100 g of glycerol monooleate in the initial oil phase. In other embodiments, the emulsion may have between about 15 to about 30 g polysorbate 60 in the initial oil phase. In yet another embodiment the composition may comprise between about 20 to about 30 g of a GENEROL sterol, in the initial oil phase.

Individual components of nanoemulsions (e.g. in an immunogenic composition of the present invention) can function both to inactivate a pathogen as well as to contribute to the non-toxicity of the emulsions. For example, the active component in BCTP, TRITON-X100, shows less ability to inactivate a virus at concentrations equivalent to 11% BCTP. Adding the oil phase to the detergent and solvent markedly reduces the toxicity of these agents in tissue culture at the same concentrations. While not being bound to any theory (an understanding of the mechanism is not necessary to practice the present invention, and the present invention is not limited to any particular mechanism), it is suggested that the nanoemulsion enhances the interaction of its components with the pathogens thereby facilitating the inactivation of the pathogen and reducing the toxicity of the individual components. Furthermore, when all the components of BCTP are combined in one composition but are not in a nanoemulsion structure, the mixture is not as effective at inactivating a pathogen as when the components are in a nanoemulsion structure.

Numerous additional embodiments presented in classes of formulations with like compositions are presented below. The following compositions recite various ratios and mixtures of active components. One skilled in the art will appreciate that the below recited formulation are exemplary and that additional formulations comprising similar percent ranges of the recited components are within the scope of the present invention.

In certain embodiments of the present invention, a nanoemulsion comprises from about 3 to 8 vol. % of TYLOXAPOL, about 8 vol. % of ethanol, about 1 vol. % of cetylpyridinium chloride (CPC), about 60 to 70 vol. % oil (e.g., soybean oil), about 15 to 25 vol. % of aqueous phase (e.g., DiH₂O or PBS), and in some formulations less than about 1 vol. % of 1N NaOH. Some of these embodiments comprise PBS. It is contemplated that the addition of 1N NaOH and/or PBS in some of these embodiments, allows the user to advantageously control the pH of the formulations, such that pH ranges from about 7.0 to about 9.0, and more preferably from about 7.1 to 8.5 are achieved. For example, one embodiment of the present invention comprises about 3 vol. % of TYLOXAPOL, about 8 vol. % of ethanol, about 1 vol. % of CPC, about 64 vol. % of soybean oil, and about 24 vol. % of DiH₂O (designated herein as Y3EC). Another similar embodiment comprises about 3.5 vol. % of TYLOXAPOL, about 8 vol. % of ethanol, and about 1 vol. % of CPC, about 64 vol. % of soybean oil, and about 23.5 vol. % of DiH₂O (designated herein as Y3.5EC). Yet another embodiment comprises about 3 vol. % of TYLOXAPOL, about 8 vol. % of ethanol, about 1 vol. % of CPC, about 0.067 vol. % of 1N NaOH, such that the pH of the formulation is about 7.1, about 64 vol. % of soybean oil, and about 23.93 vol. % of DiH₂O (designated herein as Y3EC pH 7.1). Still another embodiment comprises about 3 vol. % of TYLOXAPOL, about 8 vol. % of ethanol, about 1 vol. % of CPC, about 0.67 vol. % of 1N NaOH, such that the pH of the formulation is about 8.5, and about 64 vol. % of soybean oil, and about 23.33 vol. % of DiH₂O (designated herein as Y3EC pH 8.5). Another similar embodiment comprises about 4% TYLOXAPOL, about 8 vol. % ethanol, about 1% CPC, and about 64 vol. % of soybean oil, and about 23 vol. % of DiH₂O (designated herein as Y4EC). In still another embodiment the formulation comprises about 8% TYLOXAPOL, about 8% ethanol, about 1 vol. % of CPC, and about 64 vol. % of soybean oil, and about 19 vol. % of DiH₂O (designated herein as Y8EC). A further embodiment comprises about 8 vol. % of TYLOXAPOL, about 8 vol. % of ethanol, about 1 vol. % of CPC, about 64 vol. % of soybean oil, and about 19 vol. % of 1×PBS (designated herein as Y8EC PBS).

In some embodiments of the present invention, a nanoemulsion comprises about 8 vol. % of ethanol, and about 1 vol. % of CPC, and about 64 vol. % of oil (e.g., soybean oil), and about 27 vol. % of aqueous phase (e.g., DiH₂O or PBS) (designated herein as EC).

In some embodiments, a nanoemulsion comprises from about 8 vol. % of sodium dodecyl sulfate (SDS), about 8 vol. % of tributyl phosphate (TBP), and about 64 vol. % of oil (e.g., soybean oil), and about 20 vol. % of aqueous phase (e.g., DiH₂O or PBS) (designated herein as S8P).

In some embodiments, a nanoemulsion comprises from about 1 to 2 vol. % of TRITON X-100, from about 1 to 2 vol. % of TYLOXAPOL, from about 7 to 8 vol. % of ethanol, about 1 vol. % of cetylpyridinium chloride (CPC), about 64 to 57.6 vol. % of oil (e.g., soybean oil), and about 23 vol. % of aqueous phase (e.g., DiH₂O or PBS). Additionally, some of these formulations further comprise about 5 mM of L-alanine/Inosine, and about 10 mM ammonium chloride. Some of these formulations comprise PBS. It is contemplated that the addition of PBS in some of these embodiments, allows the user to advantageously control the pH of the formulations. For example, one embodiment of the present invention comprises about 2 vol. % of TRITON X-100, about 2 vol. % of TYLOXAPOL, about 8 vol. % of ethanol, about 1 vol. % CPC, about 64 vol. % of soybean oil, and about 23 vol. % of aqueous phase DiH₂O. In another embodiment the formulation comprises about 1.8 vol. % of TRITON X-100, about 1.8 vol. % of TYLOXAPOL, about 7.2 vol. % of ethanol, about 0.9 vol. % of CPC, about 5 mM L-alanine/Inosine, and about 10 mM ammonium chloride, about 57.6 vol. % of soybean oil, and the remainder of 1×PBS (designated herein as 90% X2Y2EC/GE).

In alternative embodiments, a nanoemulsion comprises from about 5 vol. % of TWEEN 80, from about 8 vol. % of ethanol, from about 1 vol. % of CPC, about 64 vol. % of oil (e.g., soybean oil), and about 22 vol. % of DiH₂O (designated herein as W₈₀5EC). In yet another alternative embodiment, a nanoemulsion comprises from about 5 vol. % of TWEEN 80, from about 8 vol. % of ethanol, about 64 vol. % of oil (e.g., soybean oil), and about 23 vol. % of DiH₂O (designated herein as W₈₀5E).

In some embodiments, the present invention provides a nanoemulsion comprising from about 5 vol. % of Poloxamer-407, from about 8 vol. % of ethanol, from about 1 vol. % of CPC, about 64 vol. % of oil (e.g., soybean oil), and about 22 vol. % of DiH₂O (designated herein as P₄₀₇5EC). Although an understanding of the mechanism is not necessary to practice the present invention, and the present invention is not limited to any particular mechanism, in some embodiments, a nanoemulsion comprising Poloxamer-407 does not elicit and/or augment immune responses (e.g., in the lung) in a subject. In some embodiments, various dilutions of a nanoemulsion provided herein (e.g., P₄₀₇5EC) can be utilized to treat (e.g., kill and/or inhibit growth of) bacteria. In some embodiments, undiluted nanoemulsion is utilized. In some embodiments, P₄₀₇5EC is diluted (e.g., in serial, two fold dilutions) to obtain a desired concentration of one of the constituents of the nanoemulsion (e.g., CPC).

In still other embodiments of the present invention, a nanoemulsion comprises from about 5 vol. % of TWEEN 20, from about 8 vol. % of ethanol, from about 1 vol. % of CPC, about 64 vol. % of oil (e.g., soybean oil), and about 22 vol. % of DiH₂O (designated herein as W₂₀5EC).

In still other embodiments of the present invention, a nanoemulsion comprises from about 2 to 8 vol. % of TRITON X-100, about 8 vol. % of ethanol, about 1 vol. % of CPC, about 60 to 70 vol. % of oil (e.g., soybean, or olive oil), and about 15 to 25 vol. % of aqueous phase (e.g., DiH₂O or PBS). For example, the present invention contemplates formulations comprising about 2 vol. % of TRITON X-100, about 8 vol. % of ethanol, about 64 vol. % of soybean oil, and about 26 vol. % of DiH₂O (designated herein as X2E). In other similar embodiments, a nanoemulsion comprises about 3 vol. % of TRITON X-100, about 8 vol. % of ethanol, about 64 vol. % of soybean oil, and about 25 vol. % of DiH₂O (designated herein as X3E). In still further embodiments, the formulations comprise about 4 vol. % Triton of X-100, about 8 vol. % of ethanol, about 64 vol. % of soybean oil, and about 24 vol. % of DiH₂O (designated herein as X4E). In yet other embodiments, a nanoemulsion comprises about 5 vol. % of TRITON X-100, about 8 vol. % of ethanol, about 64 vol. % of soybean oil, and about 23 vol. % of DiH₂O (designated herein as X5E). In some embodiments, a nanoemulsion comprises about 6 vol. % of TRITON X-100, about 8 vol. % of ethanol, about 64 vol. % of soybean oil, and about 22 vol. % of DiH₂O (designated herein as X6E). In still further embodiments of the present invention, a nanoemulsion comprises about 8 vol. % of TRITON X-100, about 8 vol. % of ethanol, about 64 vol. % of soybean oil, and about 20 vol. % of DiH₂O (designated herein as X8E). In still further embodiments, a nanoemulsion comprises about 8 vol. % of TRITON X-100, about 8 vol. % of ethanol, about 64 vol. % of olive oil, and about 20 vol. % of DiH₂O (designated herein as X8E O). In yet another embodiment, a nanoemulsion comprises 8 vol. % of TRITON X-100, about 8 vol. % ethanol, about 1 vol. % CPC, about 64 vol. % of soybean oil, and about 19 vol. % of DiH₂O (designated herein as X8EC).

In alternative embodiments of the present invention, a nanoemulsion comprises from about 1 to 2 vol. % of TRITON X-100, from about 1 to 2 vol. % of TYLOXAPOL, from about 6 to 8 vol. % TBP, from about 0.5 to 1.0 vol. % of CPC, from about 60 to 70 vol. % of oil (e.g., soybean), and about 1 to 35 vol. % of aqueous phase (e.g., DiH₂O or PBS). Additionally, certain of these nanoemulsions may comprise from about 1 to 5 vol. % of trypticase soy broth, from about 0.5 to 1.5 vol. % of yeast extract, about 5 mM L-alanine/Inosine, about 10 mM ammonium chloride, and from about 20-40 vol. % of liquid baby formula. In some embodiments comprising liquid baby formula, the formula comprises a casein hydrolysate (e.g., Neutramigen, or Progestimil, and the like). In some of these embodiments, a nanoemulsion further comprises from about 0.1 to 1.0 vol. % of sodium thiosulfate, and from about 0.1 to 1.0 vol. % of sodium citrate. Other similar embodiments comprising these basic components employ phosphate buffered saline (PBS) as the aqueous phase. For example, one embodiment comprises about 2 vol. % of TRITON X-100, about 2 vol. % TYLOXAPOL, about 8 vol. % TBP, about 1 vol. % of CPC, about 64 vol. % of soybean oil, and about 23 vol. % of DiH₂O (designated herein as X2Y2EC). In still other embodiments, the inventive formulation comprises about 2 vol. % of TRITON X-100, about 2 vol. % TYLOXAPOL, about 8 vol. % TBP, about 1 vol. % of CPC, about 0.9 vol. % of sodium thiosulfate, about 0.1 vol. % of sodium citrate, about 64 vol. % of soybean oil, and about 22 vol. % of DiH₂O (designated herein as X2Y2PC STS1). In another similar embodiment, a nanoemulsion comprises about 1.7 vol. % TRITON X-100, about 1.7 vol. % TYLOXAPOL, about 6.8 vol. % TBP, about 0.85% CPC, about 29.2% NEUTRAMIGEN, about 54.4 vol. % of soybean oil, and about 4.9 vol. % of DiH₂O (designated herein as 85% X2Y2PC/baby). In yet another embodiment of the present invention, a nanoemulsion comprises about 1.8 vol. % of TRITON X-100, about 1.8 vol. % of TYLOXAPOL, about 7.2 vol. % of TBP, about 0.9 vol. % of CPC, about 5 mM L-alanine/Inosine, about 10 mM ammonium chloride, about 57.6 vol. % of soybean oil, and the remainder vol. % of 0. 1×PBS (designated herein as 90% X2Y2 PC/GE). In still another embodiment, a nanoemulsion comprises about 1.8 vol. % of TRITON X-100, about 1.8 vol. % of TYLOXAPOL, about 7.2 vol. % TBP, about 0.9 vol. % of CPC, and about 3 vol. % trypticase soy broth, about 57.6 vol. % of soybean oil, and about 27.7 vol. % of DiH₂O (designated herein as 90% X2Y2PC/TSB). In another embodiment of the present invention, a nanoemulsion comprises about 1.8 vol. % TRITON X-100, about 1.8 vol. % TYLOXAPOL, about 7.2 vol. % TBP, about 0.9 vol. % CPC, about 1 vol. % yeast extract, about 57.6 vol. % of soybean oil, and about 29.7 vol. % of DiH₂O (designated herein as 90% X2Y2PC/YE).

In some embodiments of the present invention, a nanoemulsion comprises about 3 vol. % of TYLOXAPOL, about 8 vol. % of TBP, and about 1 vol. % of CPC, about 60 to 70 vol. % of oil (e.g., soybean or olive oil), and about 15 to 30 vol. % of aqueous phase (e.g., DiH₂O or PBS). In a particular embodiment of the present invention, a nanoemulsion comprises about 3 vol. % of TYLOXAPOL, about 8 vol. % of TBP, and about 1 vol. % of CPC, about 64 vol. % of soybean, and about 24 vol. % of DiH₂O (designated herein as Y3PC).

In some embodiments of the present invention, a nanoemulsion comprises from about 4 to 8 vol. % of TRITON X-100, from about 5 to 8 vol. % of TBP, about 30 to 70 vol. % of oil (e.g., soybean or olive oil), and about 0 to 30 vol. % of aqueous phase (e.g., DiH₂O or PBS). Additionally, certain of these embodiments further comprise about 1 vol. % of CPC, about 1 vol. % of benzalkonium chloride, about 1 vol. % cetylyridinium bromide, about 1 vol. % cetyldimethyletylammonium bromide, 500 μM EDTA, about 10 mM ammonium chloride, about 5 mM Inosine, and about 5 mM L-alanine. For example, in a certain preferred embodiment, a nanoemulsion comprises about 8 vol. % of TRITON X-100, about 8 vol. % of TBP, about 64 vol. % of soybean oil, and about 20 vol. % of DiH₂O (designated herein as X8P). In another embodiment of the present invention, a nanoemulsion comprises about 8 vol. % of TRITON X-100, about 8 vol. % of TBP, about 1% of CPC, about 64 vol. % of soybean oil, and about 19 vol. % of DiH₂O (designated herein as X8PC). In still another embodiment, a nanoemulsion comprises about 8 vol. % TRITON X-100, about 8 vol. % of TBP, about 1 vol. % of CPC, about 50 vol. % of soybean oil, and about 33 vol. % of DiH₂O (designated herein as ATB-X1001). In yet another embodiment, the formulations comprise about 8 vol. % of TRITON X-100, about 8 vol. % of TBP, about 2 vol. % of CPC, about 50 vol. % of soybean oil, and about 32 vol. % of DiH₂O (designated herein as ATB-X002). In some embodiments, a nanoemulsion comprises about 4 vol. % TRITON X-100, about 4 vol. % of TBP, about 0.5 vol. % of CPC, about 32 vol. % of soybean oil, and about 59.5 vol. % of DiH₂O (designated herein as 50% X8PC). In some embodiments, a nanoemulsion comprises about 8 vol. % of TRITON X-100, about 8 vol. % of TBP, about 0.5 vol. % CPC, about 64 vol. % of soybean oil, and about 19.5 vol. % of DiH₂O (designated herein as X8PC_{1/2}). In some embodiments of the present invention, a nanoemulsion comprises about 8 vol. % of TRITON X-100, about 8 vol. % of TBP, about 2 vol. % of CPC, about 64 vol. % of soybean oil, and about 18 vol. % of DiH₂O (designated herein as X8PC2). In other embodiments, a nanoemulsion comprises about 8 vol. % of TRITON X-100, about 8% of TBP, about 1% of benzalkonium chloride, about 50 vol. % of soybean oil, and about 33 vol. % of DiH₂O (designated herein as X8P BC). In an alternative embodiment of the present invention, a nanoemulsion comprises about 8 vol. % of TRITON X-100, about 8 vol. % of TBP, about 1 vol. % of cetylyridinium bromide, about 50 vol. % of soybean oil, and about 33 vol. % of DiH₂O (designated herein as X8P CPB). In another exemplary embodiment of the present invention, a nanoemulsion comprises about 8 vol. % of TRITON X-100, about 8 vol. % of TBP, about 1 vol. % of cetyldimethyletylammonium bromide, about 50 vol. % of soybean oil, and about 33 vol. % of DiH₂O (designated herein as X8P CTAB). In still further embodiments, a nanoemulsion comprises about 8 vol. % of TRITON X-100, about 8 vol. % of TBP, about 1 vol. % of CPC, about 500 μM EDTA, about 64 vol. % of soybean oil, and about 15.8 vol. % DiH₂O (designated herein as X8PC EDTA). In some embodiments, a nanoemulsion comprises 8 vol. % of TRITON X-100, about 8 vol. % of TBP, about 1 vol. % of CPC, about 10 mM ammonium chloride, about 5 mM Inosine, about 5 mM L-alanine, about 64 vol. % of soybean oil, and about 19 vol. % of DiH₂O or PBS (designated herein as X8PC GE₁ₓ). In another embodiment of the present invention, a nanoemulsion comprises about 5 vol. % of TRITON X-100, about 5% of TBP, about 1 vol. % of CPC, about 40 vol. % of soybean oil, and about 49 vol. % of DiH₂O (designated herein as X5P₅C).

In some embodiments of the present invention, a nanoemulsion comprises about 2 vol. % TRITON X-100, about 6 vol. % TYLOXAPOL, about 8 vol. % ethanol, about 64 vol. % of soybean oil, and about 20 vol. % of DiH₂O (designated herein as X2Y6E).

In an additional embodiment of the present invention, a nanoemulsion comprises about 8 vol. % of TRITON X-100, and about 8 vol. % of glycerol, about 60 to 70 vol. % of oil (e.g., soybean or olive oil), and about 15 to 25 vol. % of aqueous phase (e.g., DiH₂O or PBS). Certain nanoemulsion compositions (e.g., used to generate an immune response (e.g., for use as a vaccine) comprise about 1 vol. % L-ascorbic acid. For example, one particular embodiment comprises about 8 vol. % of TRITON X-100, about 8 vol. % of glycerol, about 64 vol. % of soybean oil, and about 20 vol. % of DiH₂O (designated herein as X8G). In still another embodiment, a nanoemulsion comprises about 8 vol. % of TRITON X-100, about 8 vol. % of glycerol, about 1 vol. % of L-ascorbic acid, about 64 vol. % of soybean oil, and about 19 vol. % of DiH₂O (designated herein as X8GV_{c}).

In still further embodiments, a nanoemulsion comprises about 8 vol. % of TRITON X-100, from about 0.5 to 0.8 vol. % of TWEEN 60, from about 0.5 to 2.0 vol. % of CPC, about 8 vol. % of TBP, about 60 to 70 vol. % of oil (e.g., soybean or olive oil), and about 15 to 25 vol. % of aqueous phase (e.g., DiH₂O or PBS). For example, in one particular embodiment a nanoemulsion comprises about 8 vol. % of TRITON X-100, about 0.70 vol. % of TWEEN 60, about 1 vol. % of CPC, about 8 vol. % of TBP, about 64 vol. % of soybean oil, and about 18.3 vol. % of DiH₂O (designated herein as X8W60PC₁). In some embodiments, a nanoemulsion comprises about 8 vol. % of TRITON X-100, about 0.71 vol. % of TWEEN 60, about 1 vol. % of CPC, about 8 vol. % of TBP, about 64 vol. % of soybean oil, and about 18.29 vol. % of DiH₂O (designated herein as W60_{0.7}X8PC). In yet other embodiments, a nanoemulsion comprises from about 8 vol. % of TRITON X-100, about 0.7 vol. % of TWEEN 60, about 0.5 vol. % of CPC, about 8 vol. % of TBP, about 64 to 70 vol. % of soybean oil, and about 18.8 vol. % of DiH₂O (designated herein as X8W60PC₂). In still other embodiments, a nanoemulsion comprises about 8 vol. % of TRITON X-100, about 0.71 vol. % of TWEEN 60, about 2 vol. % of CPC, about 8 vol. % of TBP, about 64 vol. % of soybean oil, and about 17.3 vol. % of DiH₂O. In another embodiment of the present invention, a nanoemulsion comprises about 0.71 vol. % of TWEEN 60, about 1 vol. % of CPC, about 8 vol. % of TBP, about 64 vol. % of soybean oil, and about 25.29 vol. % of DiH₂O (designated herein as W60_{0.7}PC).

In another embodiment of the present invention, a nanoemulsion comprises about 2 vol. % of dioctyl sulfosuccinate, either about 8 vol. % of glycerol, or about 8 vol. % TBP, in addition to, about 60 to 70 vol. % of oil (e.g., soybean or olive oil), and about 20 to 30 vol. % of aqueous phase (e.g., DiH₂O or PBS). For example, in some embodiments, a nanoemulsion comprises about 2 vol. % of dioctyl sulfosuccinate, about 8 vol. % of glycerol, about 64 vol. % of soybean oil, and about 26 vol. % of DiH₂O (designated herein as D2G). In another related embodiment, a nanoemulsion comprises about 2 vol. % of dioctyl sulfosuccinate, and about 8 vol. % of TBP, about 64 vol. % of soybean oil, and about 26 vol. % of DiH₂O (designated herein as D2P).

In still other embodiments of the present invention, a nanoemulsion comprises about 8 to 10 vol. % of glycerol, and about 1 to 10 vol. % of CPC, about 50 to 70 vol. % of oil (e.g., soybean or olive oil), and about 15 to 30 vol. % of aqueous phase (e.g., DiH₂O or PBS). Additionally, in certain of these embodiments, a nanoemulsion further comprises about 1 vol. % of L-ascorbic acid. For example, in some embodiments, a nanoemulsion comprises about 8 vol. % of glycerol, about 1 vol. % of CPC, about 64 vol. % of soybean oil, and about 27 vol. % of DiH₂O (designated herein as GC). In some embodiments, a nanoemulsion comprises about 10 vol. % of glycerol, about 10 vol. % of CPC, about 60 vol. % of soybean oil, and about 20 vol. % of DiH₂O (designated herein as GC10). In still another embodiment of the present invention, a nanoemulsion comprises about 10 vol. % of glycerol, about 1 vol. % of CPC, about 1 vol. % of L-ascorbic acid, about 64 vol. % of soybean or oil, and about 24 vol. % of DiH₂O (designated herein as GCV_{c}).

In some embodiments of the present invention, a nanoemulsion comprises about 8 to 10 vol. % of glycerol, about 8 to 10 vol. % of SDS, about 50 to 70 vol. % of oil (e.g., soybean or olive oil), and about 15 to 30 vol. % of aqueous phase (e.g., DiH₂O or PBS). Additionally, in certain of these embodiments, a nanoemulsion further comprise about 1 vol. % of lecithin, and about 1 vol. % of p-Hydroxybenzoic acid methyl ester. Exemplary embodiments of such formulations comprise about 8 vol. % SDS, 8 vol. % of glycerol, about 64 vol. % of soybean oil, and about 20 vol. % of DiH₂O (designated herein as S8G). A related formulation comprises about 8 vol. % of glycerol, about 8 vol. % of SDS, about 1 vol. % of lecithin, about 1 vol. % of p-Hydroxybenzoic acid methyl ester, about 64 vol. % of soybean oil, and about 18 vol. % of DiH₂O (designated herein as S8GL1B1).

In yet another embodiment of the present invention, a nanoemulsion comprises about 4 vol. % of TWEEN 80, about 4 vol. % of TYLOXAPOL, about 1 vol. % of CPC, about 8 vol. % of ethanol, about 64 vol. % of soybean oil, and about 19 vol. % of DiH₂O (designated herein as W₈₀4Y4EC).

In some embodiments of the present invention, a nanoemulsion comprises about 0.01 vol. % of CPC, about 0.08 vol. % of TYLOXAPOL, about 10 vol. % of ethanol, about 70 vol. % of soybean oil, and about 19.91 vol. % of DiH₂O (designated herein as Y.08EC.01).

In yet another embodiment of the present invention, a nanoemulsion comprises about 8 vol. % of sodium lauryl sulfate, and about 8 vol. % of glycerol, about 64 vol. % of soybean oil, and about 20 vol. % of DiH₂O (designated herein as SLS8G).

The specific formulations described above are simply examples to illustrate the variety of nanoemulsion adjuvants that find use in the present invention. The present invention contemplates that many variations of the above formulations, as well as additional nanoemulsions, find use in the methods of the present invention. Candidate emulsions can be easily tested to determine if they are suitable. First, the desired ingredients are prepared using the methods described herein, to determine if an emulsion can be formed. If an emulsion cannot be formed, the candidate is rejected. For example, a candidate composition made of 4.5% sodium thiosulfate, 0.5% sodium citrate, 10% n-butanol, 64% soybean oil, and 21% DiH₂O does not form an emulsion.

Second, the candidate emulsion should form a stable emulsion. An emulsion is stable if it remains in emulsion form for a sufficient period to allow its intended use (e.g., to generate an immune response in a subject). For example, for emulsions that are to be stored, shipped, etc., it may be desired that the composition remain in emulsion form for months to years. Typical emulsions that are relatively unstable, will lose their form within a day. For example, a candidate composition made of 8% 1-butanol, 5% TWEEN 10, 1% CPC, 64% soybean oil, and 22% DiH₂O does not form a stable emulsion. Nanoemulsions that have been shown to be stable include, but are not limited to, 8 vol. % of TRITON X-100, about 8 vol. % of TBP, about 64 vol. % of soybean oil, and about 20 vol. % of DiH₂O (designated herein as X8P); 5 vol. % of TWEEN 20, from about 8 vol. % of ethanol, from about 1 vol. % of CPC, about 64 vol. % of oil (e.g., soybean oil), and about 22 vol. % of DiH₂O (designated herein as W₂₀5EC); 0.08% Triton X-100, 0.08% Glycerol, 0.01% Cetylpyridinium Chloride, 99% Butter, and 0.83% diH₂O (designated herein as 1% X8GC Butter); 0.8% Triton X-100, 0.8% Glycerol, 0.1% Cetylpyridinium Chloride, 6.4% Soybean Oil, 1.9% diH₂O, and 90% Butter (designated herein as 10% X8GC Butter); 2% W₂₀5EC, 1% Natrosol 250 L NF, and 97% diH₂O (designated herein as 2% W₂₀5EC L GEL); 1% Cetylpyridinium Chloride, 5% TWEEN 20, 8% Ethanol, 64% 70 Viscosity Mineral Oil, and 22% diH₂O (designated herein as W₂₀5EC 70 Mineral Oil); 1% Cetylpyridinium Chloride, 5% TWEEN 20, 8% Ethanol, 64% 350 Viscosity Mineral Oil, and 22% diH₂O (designated herein as W₂₀5EC 350 Mineral Oil). In some embodiments, nanoemulsions of the present invention are stable for over a week, over a month, or over a year.

Third, the candidate emulsion should have efficacy for its intended use. For example, a nanoemuslion should inactivate (e.g., kill or inhibit growth of) a pathogen to a desired level (e.g., 1 log, 2 log, 3 log, 4 log, . . . reduction). Using the methods described herein, one is capable of determining the suitability of a particular candidate emulsion against the desired pathogen. Generally, this involves exposing the pathogen to the emulsion for one or more time periods in a side-by-side experiment with the appropriate control samples (e.g., a negative control such as water) and determining if, and to what degree, the emulsion inactivates (e.g., kills and/or neutralizes) the microorganism. For example, a candidate composition made of 1% ammonium chloride, 5% TWEEN 20, 8% ethanol, 64% soybean oil, and 22% DiH₂O was shown not to be an effective emulsion. The following candidate emulsions were shown to be effective using the methods described herein: 5% TWEEN 20, 5% Cetylpyridinium Chloride, 10% Glycerol, 60% Soybean Oil, and 20% diH₂O (designated herein as W₂₀5GC5); 1% Cetylpyridinium Chloride, 5% TWEEN 20, 10% Glycerol, 64% Soybean Oil, and 20% diH₂O (designated herein as W₂₀5GC); 1% Cetylpyridinium Chloride, 5% TWEEN 20, 8% Ethanol, 64% Olive Oil, and 22% diH₂O (designated herein as W₂₀5EC Olive Oil); 1% Cetylpyridinium Chloride, 5% TWEEN 20, 8% Ethanol, 64% Flaxseed Oil, and 22% diH₂O (designated herein as W₂₀5EC Flaxseed Oil); 1% Cetylpyridinium Chloride, 5% TWEEN 20, 8% Ethanol, 64% Corn Oil, and 22% diH₂O (designated herein as W₂₀5EC Corn Oil); 1% Cetylpyridinium Chloride, 5% TWEEN 20, 8% Ethanol, 64% Coconut Oil, and 22% diH₂O (designated herein as W₂₀5EC Coconut Oil); 1% Cetylpyridinium Chloride, 5% TWEEN 20, 8% Ethanol, 64% Cottonseed Oil, and 22% diH₂O (designated herein as W₂₀5EC Cottonseed Oil); 8% Dextrose, 5% TWEEN 10, 1% Cetylpyridinium Chloride, 64% Soybean Oil, and 22% diH₂O (designated herein as W₂₀5C Dextrose); 8% PEG 200, 5% TWEEN 10, 1% Cetylpyridinium Chloride, 64% Soybean Oil, and 22% diH₂O (designated herein as W₂₀5C PEG 200); 8% Methanol, 5% TWEEN 10, 1% Cetylpyridinium Chloride, 64% Soybean Oil, and 22% diH₂O (designated herein as W₂₀5C Methanol); 8% PEG 1000, 5% TWEEN 10, 1% Cetylpyridinium Chloride, 64% Soybean Oil, and 22% diH₂O (designated herein as W₂₀5C PEG 1000); 2% W₂₀5EC, 2% Natrosol 250H NF, and 96% diH₂O (designated herein as 2% W₂₀5EC Natrosol 2, also called 2% W₂₀5EC GEL); 2% W₂₀5EC, 1% Natrosol 250H NF, and 97% diH₂O (designated herein as 2% W₂₀5EC Natrosol 1); 2% W₂₀5EC, 3% Natrosol 250H NF, and 95% diH₂O (designated herein as 2% W₂₀5EC Natrosol 3); 2% W₂₀5EC, 0.5% Natrosol 250H NF, and 97.5% diH₂O (designated herein as 2% W₂₀5EC Natrosol 0.5); 2% W₂₀5EC, 2% Methocel A, and 96% diH₂O (designated herein as 2% W₂₀5EC Methocel A); 2% W₂₀5EC, 2% Methocel K, and 96% diH₂O (designated herein as 2% W₂₀5EC Methocel K); 2% Natrosol, 0.1% X8PC, 0.1×PBS, 5 mM L-alanine, 5 mM Inosine, 10 mM Ammonium Chloride, and diH₂O (designated herein as 0.1% X8PC/GE+2% Natrosol); 2% Natrosol, 0.8% Triton X-100, 0.8% Tributyl Phosphate, 6.4% Soybean Oil, 0.1% Cetylpyridinium Chloride, 0.1×PBS, 5 mM L-alanine, 5 mM Inosine, 10 mM Ammonium Chloride, and diH₂O (designated herein as 10% X8PC/GE+2% Natrosol); 1% Cetylpyridinium Chloride, 5% TWEEN 20, 8% Ethanol, 64% Lard, and 22% diH₂O (designated herein as W₂₀5EC Lard); 1% Cetylpyridinium Chloride, 5% TWEEN 20, 8% Ethanol, 64% Mineral Oil, and 22% diH₂O (designated herein as W₂₀5EC Mineral Oil); 0.1% Cetylpyridinium Chloride, 2% Nerolidol, 5% TWEEN 20, 10% Ethanol, 64% Soybean Oil, and 18.9% diH₂O (designated herein as W₂₀5EC_{0.1}N); 0.1% Cetylpyridinium Chloride, 2% Farnesol, 5% TWEEN 20, 10% Ethanol, 64% Soybean Oil, and 18.9% diH₂O (designated herein as W₂₀5EC_{0.1}F); 0.1% Cetylpyridinium Chloride, 5% TWEEN 20, 10% Ethanol, 64% Soybean Oil, and 20.9% diH₂O (designated herein as W₂₀5EC_{0.1}); 10% Cetylpyridinium Chloride, 8% Tributyl Phosphate, 8% Triton X-100, 54% Soybean Oil, and 20% diH₂O (designated herein as X8PC₁₀); 5% Cetylpyridinium Chloride, 8% Triton X-100, 8% Tributyl Phosphate, 59% Soybean Oil, and 20% diH₂O (designated herein as X8PC₅); 0.02% Cetylpyridinium Chloride, 0.1% TWEEN 20, 10% Ethanol, 70% Soybean Oil, and 19.88% diH₂O (designated herein as W₂₀0.1EC_{0.02}); 1% Cetylpyridinium Chloride, 5% TWEEN 20, 8% Glycerol, 64% Mobil 1, and 22% diH₂O (designated herein as W₂₀5GC Mobil 1); 7.2% Triton X-100, 7.2% Tributyl Phosphate, 0.9% Cetylpyridinium Chloride, 57.6% Soybean Oil, 0.1×PBS, 5 mM L-alanine, 5 mM Inosine, 10 mM Ammonium Chloride, and 25.87% diH₂O (designated herein as 90% X8PC/GE); 7.2% Triton X-100, 7.2% Tributyl Phosphate, 0.9% Cetylpyridinium Chloride, 57.6% Soybean Oil, 1% EDTA, 5 mM L-alanine, 5 mM Inosine, 10 mM Ammonium Chloride, 0.1×PBS, and diH₂O (designated herein as 90% X8PC/GE EDTA); and 7.2% Triton X-100, 7.2% Tributyl Phosphate, 0.9% Cetylpyridinium Chloride, 57.6% Soybean Oil, 1% Sodium Thiosulfate, 5 mM L-alanine, 5 mM Inosine, 10 mM Ammonium Chloride, 0.1×PBS, and diH₂O (designated herein as 90% X8PC/GE STS).

In preferred embodiments of the present invention, the nanoemulsions are non-toxic (e.g., to humans, plants, or animals), non-irritant (e.g., to humans, plants, or animals), and non-corrosive (e.g., to humans, plants, or animals or the environment), while retaining stability when mixed with other agents (e.g., a composition comprising an immunogen (e.g., bacteria, fungi, viruses, and spores). While a number of the above described nanoemulsions meet these qualifications, the following description provides a number of preferred non-toxic, non-irritant, non-corrosive, anti-microbial nanoemulsions of the present invention (hereinafter in this section referred to as “non-toxic nanoemulsions”).

In some embodiments the non-toxic nanoemulsions comprise surfactant lipid preparations (SLPs) for use as broad-spectrum antimicrobial agents that are effective against bacteria and their spores, enveloped viruses, and fungi. In preferred embodiments, these SLPs comprise a mixture of oils, detergents, solvents, and cationic halogen-containing compounds in addition to several ions that enhance their biocidal activities. These SLPs are characterized as stable, non-irritant, and non-toxic compounds compared to commercially available bactericidal and sporicidal agents, which are highly irritant and/or toxic.

Ingredients for use in the non-toxic nanoemulsions include, but are not limited to: detergents (e.g., TRITON X-100 (5-15%) or other members of the TRITON family, TWEEN 60 (0.5-2%) or other members of the TWEEN family, or TYLOXAPOL (1-10%)); solvents (e.g., tributyl phosphate (5-15%)); alcohols (e.g., ethanol (5-15%) or glycerol (5-15%)); oils (e.g., soybean oil (40-70%)); cationic halogen-containing compounds (e.g., cetylpyridinium chloride (0.5-2%), cetylpyridinium bromide (0.5-2%)), or cetyldimethylethyl ammonium bromide (0.5-2%)); quaternary ammonium compounds (e.g., benzalkonium chloride (0.5-2%), N-alkyldimethylbenzyl ammonium chloride (0.5-2%)); ions (calcium chloride (1 mM-40 mM), ammonium chloride (1 mM-20 mM), sodium chloride (5 mM-200 mM), sodium phosphate (1 mM-20 mM)); nucleosides (e.g., inosine (50 μM-20 mM)); and amino acids (e.g., L-alanine (50 μM-20 mM)). Emulsions are prepared, for example, by mixing in a high shear mixer for 3-10 minutes. The emulsions may or may not be heated before mixing at 82° C. for 1 hour.

Quaternary ammonium compounds for use in the present include, but are not limited to, N-alkyldimethyl benzyl ammonium saccharinate; 1,3,5-Triazine-1,3,5(2H,4H,6H)-triethanol; 1-Decanaminium, N-decyl-N,N-dimethyl-, chloride (or) Didecyl dimethyl ammonium chloride; 2-(2-(p-(Diisobuyl)cresosxy)ethoxy)ethyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl)benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl)ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl)octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethyylbenzyl ammonium chloride; and n-octadecyl dimethyl benzyl ammonium chloride.

1. Aqueous Phase

In some embodiments, the emulsion comprises an aqueous phase. In certain preferred embodiments, the emulsion comprises about 5 to 50, preferably 10 to 40, more preferably 15 to 30, vol. % aqueous phase, based on the total volume of the emulsion (although other concentrations are also contemplated). In preferred embodiments, the aqueous phase comprises water at a pH of about 4 to 10, preferably about 6 to 8. The water is preferably deionized (hereinafter “DiH₂O”). In some embodiments, the aqueous phase comprises phosphate buffered saline (PBS). In some preferred embodiments, the aqueous phase is sterile and pyrogen free.

2. Oil Phase

In some embodiments, the emulsion comprises an oil phase. In certain preferred embodiments, the oil phase (e.g., carrier oil) of the emulsion of the present invention comprises 30-90, preferably 60-80, and more preferably 60-70, vol. % of oil, based on the total volume of the emulsion (although higher and lower concentrations also find use in emulsions described herein).

The oil in the nanoemulsion adjuvant of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

In one aspect of the invention, the volatile oil in the silicone component is different than the oil in the oil phase.

In some embodiments, the oil phase comprises 3-15, and preferably 5-10 vol. % of an organic solvent, based on the total volume of the emulsion. While the present invention is not limited to any particular mechanism, it is contemplated that the organic phosphate-based solvents employed in the emulsions serve to remove or disrupt the lipids in the membranes of the pathogens. Thus, any solvent that removes the sterols or phospholipids in the microbial membranes finds use in the methods of the present invention. Suitable organic solvents include, but are not limited to, organic phosphate based solvents or alcohols. In some preferred embodiments, non-toxic alcohols (e.g., ethanol) are used as a solvent. The oil phase, and any additional compounds provided in the oil phase, are preferably sterile and pyrogen free.

3. Surfactants and Detergents

In some embodiments, the emulsions further comprises a surfactant or detergent. In some preferred embodiments, the emulsion comprises from about 3 to 15%, and preferably about 10% of one or more surfactants or detergents (although other concentrations are also contemplated). While the present invention is not limited to any particular mechanism, it is contemplated that surfactants, when present in the emulsions, help to stabilize the emulsions. Both non-ionic (non-anionic) and ionic surfactants are contemplated. Additionally, surfactants from the BRIJ family of surfactants find use in the compositions of the present invention. The surfactant can be provided in either the aqueous or the oil phase. Surfactants suitable for use with the emulsions include a variety of anionic and nonionic surfactants, as well as other emulsifying compounds that are capable of promoting the formation of oil-in-water emulsions. In general, emulsifying compounds are relatively hydrophilic, and blends of emulsifying compounds can be used to achieve the necessary qualities. In some formulations, nonionic surfactants have advantages over ionic emulsifiers in that they are substantially more compatible with a broad pH range and often form more stable emulsions than do ionic (e.g., soap-type) emulsifiers.

The surfactant in the nanoemulsion adjuvant of the invention can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference. Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂ CH₂)_{y}—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis(imidazoyl carbonyl)), nonoxynol-9, Bis(polyethylene glycol bis(imidazoyl carbonyl)), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-100, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl)benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C12-16), Alkyl dimethyl benzyl ammonium chloride (C12-18), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl)ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl)octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with an particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4,1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

The present invention is not limited to the surfactants disclosed herein. Additional surfactants and detergents useful in the compositions of the present invention may be ascertained from reference works (e.g., including, but not limited to, McCutheon's Volume 1: Emulsions and Detergents—North American Edition, 2000) and commercial sources.

4. Cationic Halogens Containing Compounds

In some embodiments, the emulsions further comprise a cationic halogen containing compound. In some preferred embodiments, the emulsion comprises from about 0.5 to 1.0 wt. % or more of a cationic halogen containing compound, based on the total weight of the emulsion (although other concentrations are also contemplated). In preferred embodiments, the cationic halogen-containing compound is preferably premixed with the oil phase; however, it should be understood that the cationic halogen-containing compound may be provided in combination with the emulsion composition in a distinct formulation. Suitable halogen containing compounds may be selected from compounds comprising chloride, fluoride, bromide and iodide ions. In preferred embodiments, suitable cationic halogen containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), and cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen-containing compound is CPC, although the compositions of the present invention are not limited to formulation with any particular cationic containing compound.

5. Germination Enhancers

In other embodiments of the present invention, the nanoemulsions further comprise a germination enhancer. In some preferred embodiments, the emulsions comprise from about 1 mM to 15 mM, and more preferably from about 5 mM to 10 mM of one or more germination enhancing compounds (although other concentrations are also contemplated). In preferred embodiments, the germination enhancing compound is provided in the aqueous phase prior to formation of the emulsion. The present invention contemplates that when germination enhancers are added to the nanoemulsion compositions, the sporicidal properties of the nanoemulsions are enhanced. The present invention further contemplates that such germination enhancers initiate sporicidal activity near neutral pH (between pH 6-8, and preferably 7). Such neutral pH emulsions can be obtained, for example, by diluting with phosphate buffer saline (PBS) or by preparations of neutral emulsions. The sporicidal activity of the nanoemulsion preferentially occurs when the spores initiate germination.

In specific embodiments, it has been demonstrated that the emulsions utilized in the vaccines of the present invention have sporicidal activity. While the present invention is not limited to any particular mechanism and an understanding of the mechanism is not required to practice the present invention, it is believed that the fusigenic component of the emulsions acts to initiate germination and before reversion to the vegetative form is complete the lysogenic component of the emulsion acts to lyse the newly germinating spore. These components of the emulsion thus act in concert to leave the spore susceptible to disruption by the emulsions. The addition of germination enhancer further facilitates the anti-sporicidal activity of the emulsions, for example, by speeding up the rate at which the sporicidal activity occurs.

Germination of bacterial endospores and fungal spores is associated with increased metabolism and decreased resistance to heat and chemical reactants. For germination to occur, the spore must sense that the environment is adequate to support vegetation and reproduction. The amino acid L-alanine stimulates bacterial spore germination (See e.g., Hills, J. Gen. Micro. 4:38 (1950); and Halvorson and Church, Bacteriol Rev. 21:112 (1957)). L-alanine and L-proline have also been reported to initiate fungal spore germination (Yanagita, Arch Mikrobiol 26:329 (1957)). Simple α-amino acids, such as glycine and L-alanine, occupy a central position in metabolism. Transamination or deamination of α-amino acids yields the glycogenic or ketogenic carbohydrates and the nitrogen needed for metabolism and growth. For example, transamination or deamination of L-alanine yields pyruvate, which is the end product of glycolytic metabolism (Embden-Meyerhof Pathway). Oxidation of pyruvate by pyruvate dehydrogenase complex yields acetyl-CoA, NADH, H⁺, and CO₂. Acetyl-CoA is the initiator substrate for the tricarboxylic acid cycle (Kreb's Cycle), which in turns feeds the mitochondrial electron transport chain. Acetyl-CoA is also the ultimate carbon source for fatty acid synthesis as well as for sterol synthesis. Simple α-amino acids can provide the nitrogen, CO₂, glycogenic and/or ketogenic equivalents required for germination and the metabolic activity that follows.

In certain embodiments, suitable germination enhancing agents of the invention include, but are not limited to, α-amino acids comprising glycine and the L-enantiomers of alanine, valine, leucine, isoleucine, serine, threonine, lysine, phenylalanine, tyrosine, and the alkyl esters thereof. Additional information on the effects of amino acids on germination may be found in U.S. Pat. No. 5,510,104; herein incorporated by reference in its entirety. In some embodiments, a mixture of glucose, fructose, asparagine, sodium chloride (NaCl), ammonium chloride (NH₄Cl), calcium chloride (CaCl₂) and potassium chloride (KCl) also may be used. In particularly preferred embodiments of the present invention, the formulation comprises the germination enhancers L-alanine, CaCl₂, Inosine and NH₄Cl. In some embodiments, the compositions further comprise one or more common forms of growth media (e.g., trypticase soy broth, and the like) that additionally may or may not itself comprise germination enhancers and buffers.

The above compounds are merely exemplary germination enhancers and it is understood that other known germination enhancers will find use in the nanoemulsions utilized in some embodiments of the present invention. A candidate germination enhancer should meet two criteria for inclusion in the compositions of the present invention: it should be capable of being associated with the emulsions disclosed herein and it should increase the rate of germination of a target spore when incorporated in the emulsions disclosed herein. One skilled in the art can determine whether a particular agent has the desired function of acting as an germination enhancer by applying such an agent in combination with the nanoemulsions disclosed herein to a target and comparing the inactivation of the target when contacted by the admixture with inactivation of like targets by the composition of the present invention without the agent. Any agent that increases germination, and thereby decreases or inhibits the growth of the organisms, is considered a suitable enhancer for use in the nanoemulsion compositions disclosed herein.

In still other embodiments, addition of a germination enhancer (or growth medium) to a neutral emulsion composition produces a composition that is useful in inactivating bacterial spores in addition to enveloped viruses, Gram negative bacteria, and Gram positive bacteria for use in the vaccine compositions of the present invention.

6. Interaction Enhancers

In still other embodiments, nanoemulsions comprise one or more compounds capable of increasing the interaction of the compositions (i.e., “interaction enhancer” (e.g., with target pathogens (e.g., the cell wall of Gram negative bacteria such as Vibrio, Salmonella, Shigella and Pseudomonas)). In preferred embodiments, the interaction enhancer is preferably premixed with the oil phase; however, in other embodiments the interaction enhancer is provided in combination with the compositions after emulsification. In certain preferred embodiments, the interaction enhancer is a chelating agent (e.g., ethylenediaminetetraacetic acid (EDTA) or ethylenebis(oxyethylenenitrilo)tetraacetic acid (EGTA) in a buffer (e.g., tris buffer)). It is understood that chelating agents are merely exemplary interaction enhancing compounds. Indeed, other agents that increase the interaction of the nanoemulsions used in some embodiments of the present invention (e.g., with microbial agents, pathogens, vaccines, etc.) are contemplated. In particularly preferred embodiments, the interaction enhancer is at a concentration of about 50 to about 250 μM. One skilled in the art will be able to determine whether a particular agent has the desired function of acting as an interaction enhancer by applying such an agent in combination with the compositions of the present invention to a target and comparing the inactivation of the target when contacted by the admixture with inactivation of like targets by the composition of the present invention without the agent. Any agent that increases the interaction of an emulsion with bacteria and thereby decreases or inhibits the growth of the bacteria, in comparison to that parameter in its absence, is considered an interaction enhancer.

In some embodiments, the addition of an interaction enhancer to nanoemulsion produces a composition that is useful in inactivating enveloped viruses, some Gram positive bacteria and some Gram negative bacteria for use in a vaccine composition.

7. Quaternary Ammonium Compounds

In some embodiments, nanoemulsions of the present invention include a quaternary ammonium containing compound. Exemplary quaternary ammonium compounds include, but are not limited to, Alkyl dimethyl benzyl ammonium chloride, didecyl dimethyl ammonium chloride, Alkyl dimethyl benzyl and dialkyl dimethyl ammonium chloride, N,N-Dimethyl-2-hydroxypropylammonium chloride polymer, Didecyl dimethyl ammonium chloride, n-Alkyl dimethyl benzyl ammonium chloride, n-Alkyl dimethyl ethylbenzyl ammonium chloride, Dialkyl dimethyl ammonium chloride, n-Alkyl dimethyl benzyl ammonium chloride, n-Tetradecyl dimethyl benzyl ammonium chloride monohydrate, n-Alkyl dimethyl benzyl ammonium chloride, Dialkyl dimethyl ammonium chloride, Hexahydro-1,3,5-tris(2-hydroxyethyl)-s-triazine, Myristalkonium chloride (and) Quat RNIUM 14, Alkyl bis(2-hydroxyethyl)benzyl ammonium chloride, Alkyl demethyl benzyl ammonium chloride, Alkyl dimethyl 3,4-dichlorobenzyl ammonium chloride, Alkyl dimethyl benzyl ammonium chloride, Alkyl dimethyl benzyl dimethylbenzyl ammonium, Alkyl dimethyl dimethybenzyl ammonium chloride, Alkyl dimethyl ethyl ammonium bromide, Alkyl dimethyl ethyl ammonium bromide, Alkyl dimethyl ethylbenzyl ammonium chloride, Alkyl dimethyl isopropylbenzyl ammonium chloride, Alkyl trimethyl ammonium chloride, Alkyl 1 or 3 benzyl-1-(2-hydroxethyl)-2-imidazolinium chloride, Dialkyl methyl benzyl ammonium chloride, Dialkyl dimethyl ammonium chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Dioctyl dimethyl ammonium chloride, Dodecyl bis(2-hydroxyethyl)octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysilyl quats, and Trimethyl dodecylbenzyl ammonium chloride.

8. Other Components

In some embodiments, a nanoemulsion adjuvant composition comprises one or more additional components that provide a desired property or functionality to the nanoemulsions. These components may be incorporated into the aqueous phase or the oil phase of the nanoemulsions and/or may be added prior to or following emulsification. For example, in some embodiments, the nanoemulsions further comprise phenols (e.g., triclosan, phenyl phenol), acidifying agents (e.g., citric acid (e.g., 1.5-6%), acetic acid, lemon juice), alkylating agents (e.g., sodium hydroxide (e.g., 0.3%)), buffers (e.g., citrate buffer, acetate buffer, and other buffers useful to maintain a specific pH), and halogens (e.g., polyvinylpyrrolidone, sodium hypochlorite, hydrogen peroxide).

Exemplary techniques for making a nanoemulsion are described below. Additionally, a number of specific, although exemplary, formulation recipes are also set forth herein.

In some embodiments, a nanoemulsion adjuvant is administered to a subject before, concurrent with or after administration of a composition comprising an immunogen (e.g., a pathogen and/or pathogen component (e.g., purified, isolated and/or recombinant pathogen peptide and/or protein)). The invention is not limited to the use of any one specific type of composition comprising an immunogen. Indeed, a variety of compositions comprising an immunogen (e.g., utilized for generating an immune response (e.g., for use as a vaccine)) may be utilized with a nanoemulsion adjuvant of the invention. In some embodiments, the composition comprising an immunogen comprises pathogens (e.g., killed pathogens), pathogen components or isolated, purified and/or recombinant parts thereof. Accordingly, in some embodiments, the composition comprising an immunogen comprises a bacterial pathogen or pathogen component including, but not limited to, Bacillus cereus, Bacillus circulans and Bacillus megaterium, Bacillus anthracis, bacteria of the genus Brucella, Vibrio cholera, Coxiella burnetii, Francisella tularensis, Chlamydia psittaci, Ricinus communis, Rickettsia prowazekii, bacterial of the genus Salmonella (e.g., S. typhi), bacteria of the genus Shigella, Cryptosporidium parvum, Burkholderia pseudomallei, Clostridium perfringens, Clostridium botulinum, Vibrio cholerae, Streptococcus pyogenes, Streptococcus agalactiae, Streptococcus pneumonia, Staphylococcus aureus, Neisseria gonorrhea, Haemophilus influenzae, Escherichia coli, Salmonella typhimurium, Shigella dysenteriae, Proteus mirabilis, Pseudomonas aeruginosa, Yersinia pestis, Yersinia enterocolitica, and Yersinia pseudotuberculosis). In other embodiments, the composition comprising an immunogen comprises a viral pathogen or pathogen component including, but not limited to, influenza A virus, avian influenza virus, H5N1 influenza virus, West Nile virus, SARS virus, Marburg virus, Arenaviruses, Nipah virus, alphaviruses, filoviruses, herpes simplex virus I, herpes simplex virus II, sendai, sindbis, vaccinia, parvovirus, human immunodeficiency virus, hepatitis B virus, hepatitis C virus, hepatitis A virus, cytomegalovirus, human papilloma virus, picornavirus, hantavirus, junin virus, and ebola virus). In still further embodiments, the composition comprising an immunogen comprises a fungal pathogen or pathogen component, including, but not limited to, Candida albicnas and parapsilosis, Aspergillus fumigatus and niger, Fusarium spp, Trychophyton spp.

In some embodiments, a nanoemulsion adjuvant is administered to a subject before, concurrent with or after administration of a vaccine containing peptides (e.g., one generally well known in the art, as exemplified by U.S. Pat. Nos. 4,601,903; 4,599,231; 4,599,230; and 4,596,792; each of which is hereby incorporated by reference).

### Formulation Techniques

Nanoemulsions of the present invention can be formed using classic emulsion forming techniques. In brief, the oil phase is mixed with the aqueous phase under relatively high shear forces (e.g., using high hydraulic and mechanical forces) to obtain an oil-in-water nanoemulsion. The emulsion is formed by blending the oil phase with an aqueous phase on a volume-to-volume basis ranging from about 1:9 to 5:1, preferably about 5:1 to 3:1, most preferably 4:1, oil phase to aqueous phase. The oil and aqueous phases can be blended using any apparatus capable of producing shear forces sufficient to form an emulsion such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, and U.S. Patent Application Nos. 20070036831, 20060251684, and 20050208083, herein incorporated by reference in their entireties.

In preferred embodiments, compositions used in the methods of the present invention comprise droplets of an oily discontinuous phase dispersed in an aqueous continuous phase, such as water. In preferred embodiments, nanoemulsions of the present invention are stable, and do not decompose even after long storage periods (e.g., greater than one or more years). Furthermore, in some embodiments, nanoemulsions are stable (e.g., in some embodiments for greater than 3 months, in some embodiments for greater than 6 months, in some embodiments for greater than 12 months, in some embodiments for greater than 18 months) after combination with an immunogen. In preferred embodiments, nanoemulsions of the present invention are non-toxic and safe when administered (e.g., via spraying or contacting mucosal surfaces, swallowed, inhaled, etc.) to a subject.

In some embodiments, a portion of the emulsion may be in the form of lipid structures including, but not limited to, unilamellar, multilamellar, and paucliamellar lipid vesicles, micelles, and lamellar phases.

In general, the preferred non-toxic nanoemulsions are characterized by the following: they are approximately 200-800 nm in diameter, although both larger and smaller diameter nanoemulsions are contemplated; the charge depends on the ingredients; they are stable for relatively long periods of time (e.g., up to two years), with preservation of their biocidal activity; they are non-irritant and non-toxic compared to their individual components due, at least in part, to their oil contents that markedly reduce the toxicity of the detergents and the solvents; they are effective at concentrations as low as, for example, 0.1%; they have antimicrobial activity against most vegetative bacteria (including Gram-positive and Gram-negative organisms), fungi, and enveloped and nonenveloped viruses in 15 minutes (e.g., 99.99% killing); and they have sporicidal activity in 1-4 hours (e.g., 99.99% killing) when produced with germination enhancers.

The present invention is not limited by the type of subject administered a composition of the present invention. The present invention is not limited by the particular formulation of a composition comprising a nanoemulsion adjuvant of the present invention. Indeed, a composition comprising a nanoemulsion of the present invention may comprise one or more different agents in addition to the nanoemulsion. These agents or cofactors include, but are not limited to, adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a composition comprising a nanoemulsion of the present invention comprises an agent and/or co-factor that enhance the ability of the nanoemulsion to induce an immune response. In some preferred embodiments, the presence of one or more co-factors or agents reduces the amount of nanoemulsion required for inducing an immune response. The present invention is not limited by the type of co-factor or agent used in a therapeutic agent of the present invention.

In some embodiments, a co-factor or agent used in a nanoemulsion composition is a bioactive agent. For example, in some embodiments, the bioactive agent may be a bioactive agent useful in a cell (e.g., a cell expressing a CFTR). Bioactive agents, as used herein, include diagnostic agents such as radioactive labels and fluorescent labels. Bioactive agents also include molecules affecting the metabolism of a cell (e.g., a cell expressing a CFTR), including peptides, nucleic acids, and other natural and synthetic drug molecules. Bioactive agents include, but are not limited to, adrenergic agent; adrenocortical steroid; adrenocortical suppressant; alcohol deterrent; aldosterone antagonist; amino acid; ammonia detoxicant; anabolic; analeptic; analgesic; androgen; anesthesia, adjunct to; anesthetic; anorectic; antagonist; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiuretic; antidote; anti-emetic; anti-epileptic; anti-estrogen; antifibrinolytic; antifungal; antiglaucoma agent; antihemophilic; antihemorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimigraine; antimitotic; antimycotic, antinauseant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; cognition adjuvant; cognition enhancer; depressant; diagnostic aid; diuretic; dopaminergic agent; ectoparasiticide; emetic; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; impotence therapy adjunct; inhibitor; keratolytic; LHRH agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; potentiator; progestin; prostaglandin; prostate growth inhibitor; prothyrotropin; psychotropic; pulmonary surface; radioactive agent; regulator; relaxant; repartitioning agent; scabicide; sclerosing agent; sedative; sedative-hypnotic; selective adenosine A1 antagonist; serotonin antagonist; serotonin inhibitor; serotonin receptor antagonist; steroid; stimulant; suppressant; symptomatic multiple sclerosis; synergist; thyroid hormone; thyroid inhibitor; thyromimetic; tranquilizer; amyotrophic lateral sclerosis agent; cerebral ischemia agent; Paget's disease agent; unstable angina agent; uricosuric; vasoconstrictor; vasodilator; vulnerary; wound healing agent; xanthine oxidase inhibitor.

Molecules useful as antimicrobials can be delivered by the methods and compositions of the invention. Antibiotics that may find use in co-administration with a composition comprising a nanoemulsion of the present invention include, but are not limited to, agents or drugs that are bactericidal and/or bacteriostatic (e.g., inhibiting replication of bacteria or inhibiting synthesis of bacterial components required for survival of the infecting organism), including, but not limited to, almecillin, amdinocillin, amikacin, amoxicillin, amphomycin, amphotericin B, ampicillin, azacitidine, azaserine, azithromycin, azlocillin, aztreonam, bacampicillin, bacitracin, benzyl penicilloyl-polylysine, bleomycin, candicidin, capreomycin, carbenicillin, cefaclor, cefadroxil, cefamandole, cefazoline, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, eveminomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezolid, linocomycin, loracarbef, magainin, meclocycline, meropenem, methacycline, methicillin, mezlocillin, minocycline, mitomycin, moenomycin, moxalactam, moxifloxacin, mycophenolic acid, nafcillin, natamycin, neomycin, netilmicin, niphimycin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamycin, BMS-284,756, L-749,345, ER-35,786, S-4661, L-786,392, MC-02479, PepS, RP 59500, and TD-6424.

In some embodiments, a composition comprising a nanoemulsion of the present invention comprises one or more mucoadhesives (See, e.g., U.S. Pat. App. No. 20050281843, hereby incorporated by reference in its entirety). The present invention is not limited by the type of mucoadhesive utilized. Indeed, a variety of mucoadhesives are contemplated to be useful in the present invention including, but not limited to, cross-linked derivatives of poly(acrylic acid) (e.g., carbopol and polycarbophil), polyvinyl alcohol, polyvinyl pyrollidone, polysaccharides (e.g., alginate and chitosan), hydroxypropyl methylcellulose, lectins, fimbrial proteins, and carboxymethylcellulose. Although an understanding of the mechanism is not necessary to practice the present invention and the present invention is not limited to any particular mechanism of action, in some embodiments, use of a mucoadhesive (e.g., in a composition comprising a nanoemulsion) enhances an immune response in a host subject due to an increase in duration and/or amount of exposure to the nanoemulsion that a subject experiences when a mucoadhesive is used compared to the duration and/or amount of exposure to the nanoemulsion in the absence of using the mucoadhesive.

In some embodiments, a composition of the present invention may comprise sterile aqueous preparations. Acceptable vehicles and solvents include, but are not limited to, water, Ringer's solution, phosphate buffered saline and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for mucosal, pulmonary, subcutaneous, intramuscular, intraperitoneal, intravenous, or administration via other routes may be found in Remington's Pharmaceutical Sciences, Mack Publishing Company, Easton, Pa.

A composition comprising a nanoemulsion adjuvant of the present invention can be used therapeutically or as a prophylactic. A composition comprising a nanoemulsion of the present invention can be administered to a subject via a number of different delivery routes and methods.

For example, the compositions of the present invention can be administered to a subject (e.g., mucosally or by pulmonary route) by multiple methods, including, but not limited to: being suspended in a solution and applied to a surface; being suspended in a solution and sprayed onto a surface using a spray applicator; being mixed with a mucoadhesive and applied (e.g., sprayed or wiped) onto a surface (e.g., mucosal or pulmonary surface); being placed on or impregnated onto a nasal and/or pulmonary applicator and applied; being applied by a controlled-release mechanism; applied using a nebulizer, aerosolized, being applied as a liposome; or being applied on a polymer.

In some embodiments, compositions of the present invention are administered mucosally (e.g., using standard techniques; See, e.g., Remington: The Science and Practice of Pharmacy, Mack Publishing Company, Easton, Pa., 19th edition, 1995 (e.g., for mucosal delivery techniques, including intranasal and pulmonary techniques), as well as European Publication No. 517,565 and Illum et al., J. Controlled Rel., 1994, 29:133-141 (e.g., for techniques of intranasal administration), each of which is hereby incorporated by reference in its entirety). The present invention is not limited by the route of administration.

Methods of intranasal and pulmonary administration are well known in the art, including the administration of a droplet or spray form of the nanoemulsion into the nasopharynx of a subject to be treated. In some embodiments, a nebulized or aerosolized composition comprising a nanoemulsion is provided. Enteric formulations such as gastro resistant capsules for oral administration, suppositories for rectal or vaginal administration may also form part of this invention. Compositions of the present invention may also be administered via the oral route. Under these circumstances, a composition comprising a nanoemulsion may comprise a pharmaceutically acceptable excipient and/or include alkaline buffers, or enteric capsules. Formulations for nasal delivery may include those with dextran or cyclodextran and saponin as an adjuvant.

In preferred embodiments, a nanoemulsion of the present invention is administered via a pulmonary delivery route and/or means. In some embodiments, an aqueous solution containing the nanoemulsion is gently and thoroughly mixed to form a solution. The solution is sterile filtered (e.g., through a 0.2 micron filter) into a sterile, enclosed vessel. Under sterile conditions, the solution is passed through an appropriately small orifice to make droplets (e.g., between 0.1 and 10 microns).

The particles may be administered using any of a number of different applicators. Suitable methods for manufacture and administration are described in the following U.S. Pat. Nos. 6,592,904; 6,518,239; 6,423,344; 6,294,204; 6,051,256 and 5,997,848 to INHALE (now NEKTAR); and U.S. Pat. No. 5,985,309; RE37,053; U.S. Pat. Nos. 6,436,443; 6,447,753; 6,503,480; and 6,635,283, to Edwards, et al. (MIT, AIR), each of which is hereby incorporated

Thus, in some embodiments, compositions of the present invention are administered by pulmonary delivery. For example, a composition of the present invention can be delivered to the lungs of a subject (e.g., a human) via inhalation (See, e.g., Adjei, et al. Pharmaceutical Research 1990; 7:565-569; Adjei, et al. Int. J. Pharmaceutics 1990; 63:135-144; Braquet, et al. J. Cardiovascular Pharmacology 1989 143-146; Hubbard, et al. (1989) Annals of Internal Medicine, Vol. III, pp. 206-212; Smith, et al. J. Clin. Invest. 1989; 84:1145-1146; Oswein, et al. “Aerosolization of Proteins”, 1990; Proceedings of Symposium on Respiratory Drug Delivery II Keystone, Colorado; Debs, et al. J. Immunol. 1988; 140:3482-3488; and U.S. Pat. No. 5,284,656 to Platz, et al, each of which are hereby incorporated by reference in its entirety). A method and composition for pulmonary delivery of drugs for systemic effect is described in U.S. Pat. No. 5,451,569 to Wong, et al., hereby incorporated by reference; See also U.S. Pat. No. 6,651,655 to Licalsi et al., hereby incorporated by reference in its entirety)). In some embodiments, a composition comprising a nanoemulsion is administered to a subject by more than one route or means (e.g., administered via pulmonary route as well as a mucosal route).

Further contemplated for use in the practice of this invention are a wide range of mechanical devices designed for pulmonary and/or nasal mucosal delivery of pharmaceutical agents including, but not limited to, nebulizers, metered dose inhalers, and powder inhalers, all of which are familiar to those skilled in the art. Some specific examples of commercially available devices suitable for the practice of this invention are the ULTRAVENT nebulizer (Mallinckrodt Inc., St. Louis, Mo.); the ACORN II nebulizer (Marquest Medical Products, Englewood, Colo.); the VENTOLIN metered dose inhaler (Glaxo Inc., Research Triangle Park, N.C.); and the SPINHALER powder inhaler (Fisons Corp., Bedford, Mass.). All such devices require the use of formulations suitable for dispensing of the therapeutic agent. Typically, each formulation is specific to the type of device employed and may involve the use of an appropriate propellant material, in addition to the usual diluents, adjuvants, surfactants, carriers and/or other agents useful in therapy. Also, the use of liposomes, microcapsules or microspheres, inclusion complexes, or other types of carriers is contemplated.

Thus, in some embodiments, a composition comprising a nanoemulsion of the present invention may be used to protect and/or treat a subject susceptible to, or suffering from, a disease by means of administering compositions comprising a nanoemulsion by mucosal, intramuscular, intraperitoneal, intradermal, transdermal, pulmonary, intravenous, subcutaneous or other route of administration described herein. Methods of systemic administration of the nanoemulsion and/or agent co-administered with the nanoemulsion may include conventional syringes and needles, or devices designed for ballistic delivery (See, e.g., WO 99/27961, hereby incorporated by reference), or needleless pressure liquid jet device (See, e.g., U.S. Pat. No. 4,596,556; U.S. Pat. No. 5,993,412, each of which are hereby incorporated by reference), or transdermal patches (See, e.g., WO 97/48440; WO 98/28037, each of which are hereby incorporated by reference). In some embodiments, the present invention provides a delivery device for systemic administration, pre-filled with the nanoemulsion composition of the present invention.

As described above, the present invention is not limited by the type of subject administered a composition of the present invention. Indeed, a wide variety of subjects are contemplated to be benefited from administration of a composition of the present invention. In preferred embodiments, the subject is a human. In some embodiments, human subjects are of any age (e.g., adults, children, infants, etc.) that have been or are likely to become exposed to a microorganism. In some embodiments, the human subjects are subjects that are more likely to receive a direct exposure to pathogenic microorganisms or that are more likely to display signs and symptoms of disease after exposure to a pathogen (e.g., subjects with CF or asthma, subjects in the armed forces, government employees, frequent travelers, persons attending or working in a school or daycare, health care workers, an elderly person, an immunocompromised person, and emergency service employees (e.g., police, fire, EMT employees)). In some embodiments, any one or all members of the general public can be administered a composition of the present invention (e.g., to prevent the occurrence or spread of disease). For example, in some embodiments, compositions and methods of the present invention are utilized to treat a group of people (e.g., a population of a region, city, state and/or country) for their own health (e.g., to prevent or treat disease) and/or to prevent or reduce the risk of disease spread from animals (e.g., birds, cattle, sheep, pigs, etc.) to humans. In some embodiments, the subjects are non-human mammals (e.g., pigs, cattle, goats, horses, sheep, or other livestock; or mice, rats, rabbits or other animal). In some embodiments, compositions and methods of the present invention are utilized in research settings (e.g., with research animals).

A composition comprising a nanoemulsion of the present invention can be administered (e.g., to a subject (e.g., via pulmonary and/or mucosal route)) as a therapeutic or as a prophylactic to prevent microbial infection.

The compositions of the present invention may additionally contain other adjunct components conventionally found in pharmaceutical compositions. Thus, for example, the compositions may contain additional, compatible, pharmaceutically-active materials such as, for example, antipyruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the nanoemulsion. In some embodiments, nanoemulsion compositions of the present invention are administered in the form of a pharmaceutically acceptable salt. When used the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives may include benzalkonium chloride (0.003-0.03% w/v); chlorobutanol (0.3-0.9% w/v); parabens (0.01-0.25% w/v) and thimerosal (0.004-0.02% w/v).

In some embodiments, a composition comprising a nanoemulsion adjuvant is co-administered with one or more antibiotics. For example, one or more antibiotics may be administered with, before and/or after administration of a composition comprising a nanoemulsion. The present invention is not limited by the type of antibiotic co-administered.

Indeed, a variety of antibiotics may be co-administered including, but not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, doxycycline, quinolones (e.g., ciprofloxacin), sulfonamides, trimethoprim, and quinolines.

A wide variety of antimicrobial agents are currently available for use in treating bacterial, fungal and viral infections. For a comprehensive treatise on the general classes of such drugs and their mechanisms of action, the skilled artisan is referred to Goodman & Gilman's “The Pharmacological Basis of Therapeutics” Eds. Hardman et al., 9th Edition, Pub. McGraw Hill, chapters 43 through 50, 1996, (herein incorporated by reference in its entirety). Generally, these agents include agents that inhibit cell wall synthesis (e.g., penicillins, cephalosporins, cycloserine, vancomycin, bacitracin); and the imidazole antifungal agents (e.g., miconazole, ketoconazole and clotrimazole); agents that act directly to disrupt the cell membrane of the microorganism (e.g., detergents such as polmyxin and colistimethate and the antifungals nystatin and amphotericin B); agents that affect the ribosomal subunits to inhibit protein synthesis (e.g., chloramphenicol, the tetracyclines, erthromycin and clindamycin); agents that alter protein synthesis and lead to cell death (e.g., aminoglycosides); agents that affect nucleic acid metabolism (e.g., the rifamycins and the quinolones); the antimetabolites (e.g., trimethoprim and sulfonamides); and the nucleic acid analogues such as zidovudine, gangcyclovir, vidarabine, and acyclovir which act to inhibit viral enzymes essential for DNA synthesis. Various combinations of antimicrobials may be employed.

The present invention also includes methods involving co-administration of a composition comprising a nanoemulsion adjuvant with one or more additional active and/or anti-infective agents. In co-administration procedures, the agents may be administered concurrently or sequentially. In one embodiment, the compositions described herein are administered prior to the other active agent(s). The pharmaceutical formulations and modes of administration may be any of those described herein. In addition, the two or more co-administered agents may each be administered using different modes (e.g., routes) or different formulations. The additional agents to be co-administered (e.g., antibiotics, a second type of nanoemulsion, etc.) can be any of the well-known agents in the art, including, but not limited to, those that are currently in clinical use.

In some embodiments, a composition comprising a nanoemulsion is administered to a subject via more than one route. For example, a subject may benefit from receiving mucosal administration (e.g., nasal administration or other mucosal routes described herein) and, additionally, receiving one or more other routes of administration (e.g., pulmonary administration (e.g., via a nebulizer, inhaler, or other methods described herein.

Other delivery systems can include time-release, delayed release or sustained release delivery systems. Such systems can avoid repeated administrations of the compositions, increasing convenience to the subject and a physician. Many types of release delivery systems are available and known to those of ordinary skill in the art. They include polymer based systems such as poly (lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109, hereby incorporated by reference. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, each of which is hereby incorporated by reference and (b) diffusional systems in which an active component permeates at a controlled rate from a polymer such as described in U.S. Pat. Nos. 3,854,480, 5,133,974 and 5,407,686, each of which is hereby incorporated by reference. In addition, pump-based hardware delivery systems can be used, some of which are adapted for implantation.

The present invention is not limited by the amount of nanoemulsion used. The amount will vary depending upon which specific nanoemulsion(s) is/are employed, and can vary from subject to subject, depending on a number of factors including, but not limited to, the species, age and general condition (e.g., health) of the subject, and the mode of administration. Procedures for determining the appropriate amount of nanoemulsion administered to a subject to induce an immune response in a subject can be readily determined using known means by one of ordinary skill in the art.

In some embodiments, it is expected that each dose (e.g., of a composition comprising a nanoemulsion comprises 1-40% nanoemulsion, in some embodiments, 20% nanoemulsion, in some embodiments less than 20% (e.g., 15%, 10%, 8%, 5% or less nanoemulsion), and in some embodiments greater than 20% nanoemulsion (e.g., 25%, 30%, 35%, 40% or more nanoemulsion). An optimal amount for a particular administration can be ascertained by one of skill in the art using standard studies involving observation of immune responses described herein.

In some embodiments, it is expected that each dose (e.g., of a composition comprising a nanoemulsion is from 0.001 to 40% or more (e.g., 0.001-10%, 0.5-5%, 1-3%, 2%, 6%, 10%, 15%, 20%, 30%, 40% or more) by weight nanoemulsion.

Similarly, the present invention is not limited by the duration of time a nanoemulsion is administered to a subject (e.g., to induce immune priming). In some embodiments, a nanoemulsion is administered one or more times (e.g. twice, three times, four times or more) daily. In some embodiments, a composition comprising a nanoemulsion is administered one or more times a day until a suitable level of immune response is generated and/or the immune response is sustained. In some embodiments, a composition comprising a nanoemulsion of the present invention is formulated in a concentrated dose that can be diluted prior to administration to a subject. For example, dilutions of a concentrated composition may be administered to a subject such that the subject receives any one or more of the specific dosages provided herein. In some embodiments, dilution of a concentrated composition may be made such that a subject is administered (e.g., in a single dose) a composition comprising 0.5-50% of the nanoemulsion present in the concentrated composition. Concentrated compositions are contemplated to be useful in a setting in which large numbers of subjects may be administered a composition of the present invention (e.g., a hospital). In some embodiments, a composition comprising a nanoemulsion of the present invention (e.g., a concentrated composition) is stable at room temperature for more than 1 week, in some embodiments for more than 2 weeks, in some embodiments for more than 3 weeks, in some embodiments for more than 4 weeks, in some embodiments for more than 5 weeks, and in some embodiments for more than 6 weeks.

Dosage units may be proportionately increased or decreased based on several factors including, but not limited to, the weight, age, and health status of the subject. In addition, dosage units may be increased or decreased for subsequent administrations.

It is contemplated that the compositions and methods of the present invention will find use in various settings, including research settings. For example, compositions and methods of the present invention also find use in studies of the immune system (e.g., characterization of adaptive immune responses (e.g., protective immune responses (e.g., mucosal or systemic immunity))). Uses of the compositions and methods provided by the present invention encompass human and non-human subjects and samples from those subjects, and also encompass research applications using these subjects. Compositions and methods of the present invention are also useful in studying and optimizing nanoemulsions, immunogens, and other components and for screening for new components. Thus, it is not intended that the present invention be limited to any particular subject and/or application setting.

The formulations can be tested in vivo in a number of animal models developed for the study of pulmonary, mucosal and other routes of delivery. As is readily apparent, the compositions of the present invention are useful for preventing and/or treating a wide variety of diseases and infections caused by viruses, bacteria, parasites, and fungi. Not only can the compositions be used prophylactically or therapeutically, as described above, the compositions can also be used in order to prepare antibodies, both polyclonal and monoclonal (e.g., for diagnostic purposes), as well as for immunopurification of an antigen of interest.

In one embodiment, the adjuvant mixtures of the present invention are useful for the production of immunogenic compositions that can be used to generate antigen-specific antibodies that are useful in the specific identification of that antigen in an immunoassay according to a diagnostic embodiment. Such immunoassays include enzyme-linked immunosorbant assays (ELISA), RIAs and other non-enzyme linked antibody binding assays or procedures known in the art. In ELISA assays, the antigen-specific antibodies are immobilized onto a selected surface; for example, the wells of a polystyrene microtiter plate. After washing to remove incompletely adsorbed antibodies, a nonspecific protein, such as a solution of bovine serum albumin (BSA) or casein, that is known to be antigenically neutral with regard to the test sample may be bound to the selected surface. This allows for blocking of nonspecific adsorption sites on the immobilizing surface and thus reduces the background caused by nonspecific bindings of antigens onto the surface. The immobilizing surface is then contacted with a sample, such as clinical or biological materials, to be tested in a manner conducive to immune complex (antigen/antibody) formation. This may include diluting the sample with diluents, such as BSA, bovine gamma globulin (BGG) and/or phosphate buffered saline (PBS)/Tween. The sample is then allowed to incubate for from about 2 to 4 hours, at temperatures such as of the order of about 25-37° C. Following incubation, the sample-contacted surface is washed to remove non-immunocomplexed material. The washing procedure may include washing with a solution such as PBS/Tween, or a borate buffer.

Following formation of specific immunocomplexes between the antigen in the test sample and the bound antigen-specific antibodies, and subsequent washing, the occurrence, and even amount, of immunocomplex formation may be determined by subjecting the immunocomplex to a second antibody having specificity for the antigen. To provide detecting means, the second antibody may have an associated activity, such as an enzymatic activity, that will generate, for example, a color development upon incubating with an appropriate chromogenic substrate. Quantification may then achieved by measuring the degree of color generation using, for example, a visible spectra spectrophotometer. In an additional embodiment, the present invention includes a diagnostic kit comprising antigen-specific antibodies generated by immunization of a host with immunogenic compositions produced according to the present invention.

In some embodiments, the present invention provides a kit comprising a composition comprising a nanoemulsion adjuvant. In some embodiments, the kit further provides a device for administering the composition. The present invention is not limited by the type of device included in the kit. In some embodiments, the device is configured for pulmonary application of the composition of the present invention (e.g., a nasal inhaler or nasal mister). In some embodiments, a kit comprises a composition comprising a nanoemulsion in a concentrated form (e.g., that can be diluted prior to administration to a subject).

In some embodiments, all kit components are present within a single container (e.g., vial or tube). In some embodiments, each kit component is located in a single container (e.g., vial or tube (e.g., a nanoemulsion adjuvant is present in one container and an immunogen is present in a second, separate container)). In some embodiments, one or more kit components are located in a single container (e.g., vial or tube) with other components of the same kit being located in a separate container (e.g., vial or tube). In some embodiments, a kit comprises a buffer. In some embodiments, the kit further comprises instructions for use.

### Animal Models

In some embodiments, nanoemulsion adjuvant compositions (e.g., for generating an immune response (e.g., for use as an adjuvant and/or vaccine) are tested in animal models of infectious diseases. The use of well-developed animal models provides a method of measuring the effectiveness and safety of a vaccine before administration to human subjects. Exemplary animal models of disease are shown in Table 2. These animals are commercially available (e.g., from Jackson Laboratories Charles River; Portage, Mich.).

Animal models of Bacillus cereus (closely related to Bacillus anthracis) are utilized to test Anthrax vaccines of the present invention. Both bacteria are spore forming Gram positive rods and the disease syndrome produced by each bacteria is largely due to toxin production and the effects of these toxins on the infected host (Brown et al., J. Bact., 75:499 (1958); Burdon and Wende, J. Infect Dis., 107:224 (1960); Burdon et al., J. Infect. Dis., 117:307 (1967)). Bacillus cereus infection mimics the disease syndrome caused by Bacillus anthracis. Mice are reported to rapidly succumb to the effects of B. cereus toxin and are a useful model for acute infection. Guinea pigs develop a skin lesion subsequent to subcutaneous infection with B. cereus that resembles the cutaneous form of anthrax.

Clostridium perfringens infection in both mice and guinea pigs has been used as a model system for the in vivo testing of antibiotic drugs (Stevens et al., Antimicrob. Agents Chemother., 31:312 (1987); Stevens et al., J. Infect. Dis., 155:220 (1987); Alttemeier et al., Surgery, 28:621 (1950); Sandusky et al., Surgery, 28:632 (1950)). Clostridium tetani is well known to infect and cause disease in a variety of mammalian species. Mice, guinea pigs, and rabbits have all been used experimentally (Willis, Topley and Wilson's Principles of Bacteriology, Virology and Immunity. Wilson, G., A. Miles, and M. T. Parker, eds. pages 442-475 1983).

Vibrio cholerae infection has been successfully initiated in mice, guinea pigs, and rabbits. According to published reports it is preferred to alter the normal intestinal bacterial flora for the infection to be established in these experimental hosts. This is accomplished by administration of antibiotics to suppress the normal intestinal flora and, in some cases, withholding food from the animals (Butterton et al., Infect. Immun., 64:4373 (1996); Levine et al., Microbiol. Rev., 47:510 (1983); Finkelstein et al., J. Infect. Dis., 114:203 (1964); Freter, J. Exp. Med., 104:411 (1956); and Freter, J. Infect. Dis., 97:57 (1955)).

Shigella flexnerii infection has been successfully initiated in mice and guinea pigs. As is the case with vibrio infections, it is preferred that the normal intestinal bacterial flora be altered to aid in the establishment of infection in these experimental hosts. This is accomplished by administration of antibiotics to suppress the normal intestinal flora and, in some cases, withholding food from the animals (Levine et al., Microbiol. Rev., 47:510 (1983); Freter, J. Exp. Med., 104:411 (1956); Formal et al., J. Bact., 85:119 (1963); LaBrec et al., J. Bact. 88:1503 (1964); Takeuchi et al., Am. J. Pathol., 47:1011 (1965)).

Mice and rats have been used extensively in experimental studies with Salmonella typhimurium and Salmonella enteriditis (Naughton et al., J. Appl. Bact., 81:651 (1996); Carter and Collins, J. Exp. Med., 139:1189 (1974); Collins, Infect. Immun., 5:191 (1972); Collins and Carter, Infect. Immun., 6:451 (1972)).

Mice and rats are well established experimental models for infection with Sendai virus (Jacoby et al., Exp. Gerontol., 29:89 (1994); Massion et al., Am. J. Respir. Cell Mol. Biol. 9:361 (1993); Castleman et al., Am. J. Path., 129:277 (1987); Castleman, Am. J. Vet. Res., 44:1024 (1983); Mims and Murphy, Am. J. Path., 70:315 (1973)).

Sindbis virus infection of mice is usually accomplished by intracerebral inoculation of newborn mice. Alternatively, weanling mice are inoculated subcutaneously in the footpad (Johnson et al., J. Infect. Dis., 125:257 (1972); Johnson, Am. J. Path., 46:929 (1965)).

It is preferred that animals are housed for 3-5 days to rest from shipping and adapt to new housing environments before use in experiments. At the start of each experiment, control animals are sacrificed and tissue is harvested to establish baseline parameters. Animals are anesthetized by any suitable method (e.g., including, but not limited to, inhalation of Isofluorane for short procedures or ketamine/xylazine injection for longer procedure).

TABLE 2
Animal Models of Infectious Diseases

| | | Experimental | | | |
|---|---|---|---|---|---|
| | Experimental | Animal | | | Route of |
| Microorganism | Animal Species | Strains | Sex | Age | Infection |
| Francisella | mice | BALB/C | M | 6 W | Intraperitoneal |
| philomiraga | | | | | |
| Neisseria | mice | BALB/C | F | 6-10 W | Intraperitoneal |
| meningitidis | rats | COBS/CD | M/F | 4 D | Intranasal |
| Streptococcus | mice | BALB/C | F | 6 W | Intranasal |
| pneumoniae | rats | COBS/CD | M | 6-8 W | Intranasal |
| | guinea Pigs | Hartley | M/F | 4-5 W | Intranasal |
| Yersinia | mice | BALB/C | F | 6 W | Intranasal |
| pseudotuberculosis | | | | | |
| Influenza virus | mice | BALB/C | F | 6 W | Intranasal |
| Sendai virus | mice | CD-1 | F | 6 W | Intranasal |
| | rats | Sprague- | M | 6-8 W | Intranasal |
| | | Dawley | | | |
| Sindbis | mice | CD-1 | M/F | 1-2 D | Intracerebral/SC |
| Vaccinia | mice | BALB/C | F | 2-3 W | Intradermal |

### Assays for Evaluation of Adjuvants and Vaccines

In some embodiments, nanoemulsion adjuvants and/or vaccines comprising the same are evaluated using one of several suitable model systems. For example, cell-mediated immune responses can be evaluated in vitro. In addition, an animal model may be used to evaluate in vivo immune response and immunity to pathogen challenge. Any suitable animal model may be utilized, including, but not limited to, those disclosed in Table 2.

Before testing a nanoemulsion vaccine in an animal system, the amount of exposure of the pathogen to a nanoemulsion sufficient to inactivate the pathogen is investigated. It is contemplated that pathogens such as bacterial spores require longer periods of time for inactivation by the nanoemulsion in order to be sufficiently neutralized to allow for immunization. The time period required for inactivation may be investigated using any suitable method, including, but not limited to, those described in the illustrative examples below.

In addition, the stability of emulsion-developed vaccines is evaluated, particularly over time and storage condition, to ensure that vaccines are effective long-term. The ability of other stabilizing materials (e.g., dendritic polymers) to enhance the stability and immunogenicity of vaccines is also evaluated.

Once a given nanoemulsion/pathogen vaccine has been formulated to result in pathogen inactivation, the ability of the vaccine to elicit an immune response and provide immunity is optimized. Non-limiting examples of methods for assaying vaccine effectiveness are described in Example 14 below. For example, the timing and dosage of the vaccine can be varied and the most effective dosage and administration schedule determined. The level of immune response is quantitated by measuring serum antibody levels. In addition, in vitro assays are used to monitor proliferation activity by measuring H³-thymidine uptake. In addition to proliferation, Th1 and Th2 cytokine responses (e.g., including but not limited to, levels of include IL-2, TNF-α, IFN-γ, IL-4, IL-6, IL-11, IL-12, etc.) are measured to qualitatively evaluate the immune response.

Finally, animal models are utilized to evaluate the effect of a nanoemulsion mucosal vaccine. Purified pathogens are mixed in emulsions (or emulsions are contact with a pre-infected animal), administered, and the immune response is determined. The level of protection is then evaluated by challenging the animal with the specific pathogen and subsequently evaluating the level of disease symptoms. The level of immunity is measured over time to determine the necessity and spacing of booster immunizations.

### Therapeutics and Prophylactics

Furthermore, in preferred embodiments, a nanoemulsion adjuvant composition of the present invention induces (e.g., when administered to a subject) innate and adaptive/acquired immune responses (e.g., both systemic and mucosal immunity). Thus, in some preferred embodiments, administration of a composition of the present invention to a subject results in protection against an exposure (e.g., a mucosal exposure) to a pathogen. Although an understanding of the mechanism is not necessary to practice the present invention and the present invention is not limited to any particular mechanism of action, mucosal administration (e.g., vaccination) provides protection against pathogen infection (e.g., that initiates at a mucosal surface). Although it has heretofore proven difficult to stimulate secretory IgA responses and protection against pathogens that invade at mucosal surfaces (See, e.g., Mestecky et al, Mucosal Immunology. 3ed edn. (Academic Press, San Diego, 2005)), the present invention provides compositions and methods for stimulating mucosal immunity (e.g., a protective IgA response) from a pathogen in a subject.

In some embodiments, the present invention provides a composition (e.g., a composition comprising a NE and immunogenic protein antigens to serve as a mucosal vaccine. This material can be produced with NE and pathogen derived protein. The ability to produce this formulation rapidly and administer it via mucosal (e.g., nasal or vaginal) instillation provides a vaccine that can be used in large-scale administrations (e.g., to a population of a town, village, city, state or country).

In some preferred embodiments, the present invention provides a composition for generating an immune response comprising a NE and an immunogen (e.g., a purified, isolated or synthetic protein or derivative, variant, or analogue thereof; or, one or more serotypes of pathogens inactivated by the nanoemulsion). When administered to a subject, a composition of the present invention stimulates an immune response against the immunogen/pathogen within the subject. Although an understanding of the mechanism is not necessary to practice the present invention and the present invention is not limited to any particular mechanism of action, in some embodiments, generation of an immune response (e.g., resulting from administration of a composition comprising a nanoemulsion and an immunogen) stimulates innate and/or adaptive/acquired immune responses that provides total or partial immunity to the subject (e.g., from signs, symptoms or conditions of a disease (e.g., caused by the pathogen)). Without being bound to any specific theory, protection and/or immunity from disease (e.g., the ability of a subject's immune system to prevent or attenuate (e.g., suppress) a sign, symptom or condition of disease) after exposure to an immunogenic composition of the present invention is due to adaptive (e.g., acquired) immune responses (e.g., immune responses mediated by B and T cells following exposure to a NE comprising an immunogen of the present invention (e.g., immune responses that exhibit increased specificity and reactivity towards the pathogen). Thus, in some embodiments, the compositions and methods of the present invention are used prophylactically or therapeutically to prevent or attenuate a sign, symptom or condition associated with the pathogen.

In some embodiments, a nanoemulsion adjuvant is administered alone. In some embodiments, a nanoemulsion adjuvant comprises one or more other agents (e.g., a pharmaceutically acceptable carrier, other adjuvant, excipient, and the like). In some embodiments, a nanoemulsion adjuvant is administered in a manner to induce a humoral immune response. In some embodiments, a nanoemulsion adjuvant is administered in a manner to induce a cellular (e.g., cytotoxic T lymphocyte) immune response, rather than a humoral response. In some embodiments, a nanoemulsion adjuvant induces both a cellular and humoral immune response.

The present invention is not limited by the particular formulation of a composition comprising a nanoemulsion adjuvant (e.g., independently or together with an immunogen) of the present invention. Indeed, a composition comprising a nanoemulsion adjuvant of the present invention may comprise one or more different agents in addition to the nanoemulsion adjuvant. These agents or cofactors include, but are not limited to, additional adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a composition comprising a nanoemulsion adjuvant of the present invention comprises an agent and/or co-factor that enhance the ability of the nanoemulsion adjuvant to induce an immune response. In some preferred embodiments, the presence of one or more co-factors or agents reduces the amount of nanoemulsion adjuvant required for induction of an immune response (e.g., a protective immune response (e.g., protective immunization)). In some embodiments, the presence of one or more co-factors or agents can be used to skew the immune response towards a cellular (e.g., T cell mediated) or humoral (e.g., antibody mediated) immune response. The present invention is not limited by the type of co-factor or agent used in a therapeutic agent of the present invention.

Adjuvants are described in general in Vaccine Design—the Subunit and Adjuvant Approach, edited by Powell and Newman, Plenum Press, New York, 1995. The present invention is not limited by the type of adjuvant utilized (e.g., for use in a composition (e.g., pharmaceutical composition) comprising a nanoemulsion adjuvant). For example, in some embodiments, suitable adjuvants include an aluminium salt such as aluminium hydroxide gel (alum) or aluminium phosphate. In some embodiments, an adjuvant may be a salt of calcium, iron or zinc, or may be an insoluble suspension of acylated tyrosine, or acylated sugars, cationically or anionically derivatised polysaccharides, or polyphosphazenes.

In some embodiments, a composition comprising a nanoemulsion adjuvant described herein (e.g., with or without an immunogen) comprises one or more additional adjuvants that induce and/or skew toward a Th1-type response. However, in other embodiments, it will be preferred that a composition comprising a nanoemulsion adjuvant described herein (e.g., with or without an immunogen) comprises one or more additional adjuvants that induce and/or skew toward a Th2-type response.

In general, an immune response is generated to an antigen through the interaction of the antigen with the cells of the immune system. Immune responses may be broadly categorized into two categories: humoral and cell mediated immune responses (e.g., traditionally characterized by antibody and cellular effector mechanisms of protection, respectively). These categories of response have been termed Th1-type responses (cell-mediated response), and Th2-type immune responses (humoral response).

Stimulation of an immune response can result from a direct or indirect response of a cell or component of the immune system to an intervention (e.g., exposure to an immunogen). Immune responses can be measured in many ways including activation, proliferation or differentiation of cells of the immune system (e.g., B cells, T cells, dendritic cells, APCs, macrophages, NK cells, NKT cells etc.); up-regulated or down-regulated expression of markers and cytokines; stimulation of IgA, IgM, or IgG titer; splenomegaly (including increased spleen cellularity); hyperplasia and mixed cellular infiltrates in various organs. Other responses, cells, and components of the immune system that can be assessed with respect to immune stimulation are known in the art.

Although an understanding of the mechanism is not necessary to practice the present invention and the present invention is not limited to any particular mechanism of action, in some embodiments, compositions and methods of the present invention induce expression and secretion of cytokines (e.g., by macrophages, dendritic cells and CD4+ T cells (See, e.g., Examples 11-12). Modulation of expression of a particular cytokine can occur locally or systemically. It is known that cytokine profiles can determine T cell regulatory and effector functions in immune responses. In some embodiments, Th1-type cytokines can be induced, and thus, the immunostimulatory compositions of the present invention can promote a Th1 type antigen-specific immune response including cytotoxic T-cells. However in other embodiments, Th2-type cytokines can be induced thereby promoting a Th2 type antigen-specific immune response.

Cytokines play a role in directing the T cell response. Helper (CD4+) T cells orchestrate the immune response of mammals through production of soluble factors that act on other immune system cells, including B and other T cells. Most mature CD4+ T helper cells express one of two cytokine profiles: Th1 or Th2. Th1-type CD4+ T cells secrete IL-2, IL-3, IFN-γ, GM-CSF and high levels of TNF-α. Th2 cells express IL-3, IL-4, IL-5, IL-6, IL-9, IL-10, IL-13, GM-CSF and low levels of TNF-α. Th1 type cytokines promote both cell-mediated immunity, and humoral immunity that is characterized by immunoglobulin class switching to IgG2a in mice and IgG1 in humans. Th1 responses may also be associated with delayed-type hypersensitivity and autoimmune disease. Th2 type cytokines induce primarily humoral immunity and induce class switching to IgG1 and IgE. The antibody isotypes associated with Th1 responses generally have neutralizing and opsonizing capabilities whereas those associated with Th2 responses are associated more with allergic responses.

Several factors have been shown to influence skewing of an immune response towards either a Th1 or Th2 type response. The best characterized regulators are cytokines IL-12 and IFN-γ are positive Th1 and negative Th2 regulators. IL-12 promotes IFN-γ production, and IFN-γ provides positive feedback for IL-12. IL-4 and IL-10 appear important for the establishment of the Th2 cytokine profile and to down-regulate Th1 cytokine production.

Thus, in some preferred embodiments, the present invention provides a method of stimulating a Th1-type immune response in a subject comprising administering to a subject a composition comprising a nanoemulsion adjuvant described herein (e.g., with or without an immunogen). However, in other preferred embodiments, the present invention provides a method of stimulating a Th2-type immune response in a subject comprising administering to a subject a composition comprising a nanoemulsion adjuvant described herein (e.g., with or without an immunogen). In further preferred embodiments, additional adjuvants can be used (e.g., can be co-administered with a nanoemulsion adjuvant composition of the present invention) to skew an immune response toward either a Th1 or Th2 type immune response. For example, adjuvants that induce Th2 or weak Th1 responses include, but are not limited to, alum, saponins, and SB-As4. Adjuvants that induce Th1 responses include but are not limited to MPL, MDP, ISCOMS, IL-12, IFN-γ, and SB-AS2.

Several other types of Th1-type immunogens can be used (e.g., as an adjuvant) in compositions and methods of the present invention. These include, but are not limited to, the following. In some embodiments, monophosphoryl lipid A (e.g., in particular 3-de-O-acylated monophosphoryl lipid A (3D-MPL)), is used. 3D-MPL is a well known adjuvant manufactured by Ribi Immunochem, Montana. Chemically it is often supplied as a mixture of 3-de-O-acylated monophosphoryl lipid A with either 4, 5, or 6 acylated chains. In some embodiments, diphosphoryl lipid A, and 3-O-deacylated variants thereof are used. Each of these immunogens can be purified and prepared by methods described in GB 2122204B, hereby incorporated by reference in its entirety. Other purified and synthetic lipopolysaccharides have been described (See, e.g., U.S. Pat. No. 6,005,099 and EP 0 729 473; Hilgers et al., 1986, Int. Arch. Allergy. Immunol., 79(4):392-6; Hilgers et al., 1987, Immunology, 60(1):141-6; and EP 0 549 074, each of which is hereby incorporated by reference in its entirety). In some embodiments, 3D-MPL is used in the form of a particulate formulation (e.g., having a small particle size less than 0.2 μm in diameter, described in EP 0 689 454, hereby incorporated by reference in its entirety).

In some embodiments, saponins are used as an immunogen (e.g., Th1-type adjuvant) in a composition of the present invention. Saponins are well known adjuvants (See, e.g., Lacaille-Dubois and Wagner (1996) Phytomedicine vol 2 pp 363-386). Examples of saponins include Quil A (derived from the bark of the South American tree Quillaja Saponaria Molina), and fractions thereof (See, e.g., U.S. Pat. No. 5,057,540; Kensil, Crit. Rev Ther Drug Carrier Syst, 1996, 12 (1-2):1-55; and EP 0 362 279, each of which is hereby incorporated by reference in its entirety). Also contemplated to be useful in the present invention are the haemolytic saponins QS7, QS17, and QS21 (HPLC purified fractions of Quil A; See, e.g., Kensil et al. (1991). J. Immunology 146, 431-437, U.S. Pat. No. 5,057,540; WO 96/33739; WO 96/11711 and EP 0 362 279, each of which is hereby incorporated by reference in its entirety). Also contemplated to be useful are combinations of QS21 and polysorbate or cyclodextrin (See, e.g., WO 99/10008, hereby incorporated by reference in its entirety.

In some embodiments, an immunogenic oligonucleotide containing unmethylated CpG dinucleotides (“CpG”) is used as an adjuvant in the present invention. CpG is an abbreviation for cytosine-guanosine dinucleotide motifs present in DNA. CpG is known in the art as being an adjuvant when administered by both systemic and mucosal routes (See, e.g., WO 96/02555, EP 468520, Davis et al., J. Immunol, 1998, 160(2):870-876; McCluskie and Davis, J. Immunol., 1998, 161(9):4463-6; and U.S. Pat. App. No. 20050238660, each of which is hereby incorporated by reference in its entirety). For example, in some embodiments, the immunostimulatory sequence is Purine-Purine-C-G-pyrimidine-pyrimidine; wherein the CG motif is not methylated.

Although an understanding of the mechanism is not necessary to practice the present invention and the present invention is not limited to any particular mechanism of action, in some embodiments, the presence of one or more CpG oligonucleotides activate various immune subsets including natural killer cells (which produce IFN-γ) and macrophages. In some embodiments, CpG oligonucleotides are formulated into a composition of the present invention for inducing an immune response. In some embodiments, a free solution of CpG is co-administered together with an antigen (e.g., present within a NE solution (See, e.g., WO 96/02555; hereby incorporated by reference). In some embodiments, a CpG oligonucleotide is covalently conjugated to an antigen (See, e.g., WO 98/16247, hereby incorporated by reference), or formulated with a carrier such as aluminium hydroxide (See, e.g., Brazolot-Millan et al., Proc. Natl. Acad Sci., USA, 1998, 95(26), 15553-8).

In some embodiments, adjuvants such as Complete Freunds Adjuvant and Incomplete Freunds Adjuvant, cytokines (e.g., interleukins (e.g., IL-2, IFN-γ, IL-4, etc.), macrophage colony stimulating factor, tumor necrosis factor, etc.), detoxified mutants of a bacterial ADP-ribosylating toxin such as a cholera toxin (CT), a pertussis toxin (PT), or an E. Coli heat-labile toxin (LT), particularly LT-K63 (where lysine is substituted for the wild-type amino acid at position 63) LT-R72 (where arginine is substituted for the wild-type amino acid at position 72), CT-S 109 (where serine is substituted for the wild-type amino acid at position 109), and PT-K9/G129 (where lysine is substituted for the wild-type amino acid at position 9 and glycine substituted at position 129) (See, e.g., WO93/13202 and WO92/19265, each of which is hereby incorporated by reference), and other immunogenic substances (e.g., that enhance the effectiveness of a composition of the present invention) are used with a composition comprising a NE and immunogen of the present invention.

Additional examples of adjuvants that find use in the present invention include poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Adjuvants may be added to a composition comprising a nanoemulsion adjuvant and an immunogen, or, the adjuvant may be formulated with carriers, for example liposomes, or metallic salts (e.g., aluminium salts (e.g., aluminium hydroxide)) prior to combining with or co-administration with a composition comprising a nanoemulsion adjuvant and an immunogen.

In some embodiments, a composition comprising a nanoemulsion adjuvant and an immunogen comprises a single additional adjuvant. In other embodiments, a composition comprising a nanoemulsion adjuvant and an immunogen comprises two or more additional adjuvants (See, e.g., WO 94/00153; WO 95/17210; WO 96/33739; WO 98/56414; WO 99/12565; WO 99/11241; and WO 94/00153, each of which is hereby incorporated by reference in its entirety).

In some embodiments, a composition comprising a NE adjuvant described herein (e.g., with or without an immunogen) of the present invention comprises one or more mucoadhesives (See, e.g., U.S. Pat. App. No. 20050281843, hereby incorporated by reference in its entirety). The present invention is not limited by the type of mucoadhesive utilized. Indeed, a variety of mucoadhesives are contemplated to be useful in the present invention including, but not limited to, cross-linked derivatives of poly(acrylic acid) (e.g., carbopol and polycarbophil), polyvinyl alcohol, polyvinyl pyrollidone, polysaccharides (e.g., alginate and chitosan), hydroxypropyl methylcellulose, lectins, fimbrial proteins, and carboxymethylcellulose. In some embodiments, one or more components of the NE adjuvant function as a mucoadhesive (e.g., individually, or in combination with other components of the NE adjuvant). Although an understanding of the mechanism is not necessary to practice the present invention and the present invention is not limited to any particular mechanism of action, in some embodiments, use of a mucoadhesive (e.g., in a composition comprising a NE and immunogen) enhances induction of an immune response (e.g., an innate and/or adaptive immune response) in a subject (e.g., a subject administered a composition of the present invention) due to an increase in duration and/or amount of exposure to NE adjuvant and/or immunogen that a subject experiences when a mucoadhesive is used compared to the duration and/or amount of exposure to an immunogen in the absence of using the mucoadhesive).

In some embodiments, a composition of the present invention may comprise sterile aqueous preparations. Acceptable vehicles and solvents include, but are not limited to, water, Ringer's solution, phosphate buffered saline and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for mucosal, subcutaneous, intramuscular, intraperitoneal, intravenous, or administration via other routes may be found in Remington's Pharmaceutical Sciences, Mack Publishing Company, Easton, Pa.

A composition comprising a nanoemulsion adjuvant of the invention (with or without immunogen)can be used therapeutically (e.g., to enhance an immune response) or as a prophylactic (e.g., for immunization (e.g., to prevent signs or symptoms of disease)). A composition comprising a nanoemulsion adjuvant (with or without immunogen) can be administered to a subject via a number of different delivery routes and methods.

For example, the compositions of the invention can be administered to a subject (e.g., mucosally (e.g., nasal mucosa, vaginal mucosa, etc.)) by multiple methods, including, but not limited to: being suspended in a solution and applied to a surface; being suspended in a solution and sprayed onto a surface using a spray applicator; being mixed with a mucoadhesive and applied (e.g., sprayed or wiped) onto a surface (e.g., mucosal surface); being placed on or impregnated onto a nasal and/or vaginal applicator and applied; being applied by a controlled-release mechanism; being applied as a liposome; or being applied on a polymer.

In some preferred embodiments, compositions of the invention are administered mucosally (e.g., using standard techniques; See, e.g., Remington: The Science and Practice of Pharmacy, Mack Publishing Company, Easton, Pa., 19th edition, 1995 (e.g., for mucosal delivery techniques, including intranasal, pulmonary, vaginal and rectal techniques), as well as European Publication No. 517,565 and Illum et al., J. Controlled Rel., 1994, 29:133-141 (e.g., for techniques of intranasal administration), each of which is hereby incorporated by reference in its entirety). Alternatively, the compositions of the present invention may be administered dermally or transdermally, using standard techniques (See, e.g., Remington: The Science arid Practice of Pharmacy, Mack Publishing Company, Easton, Pa., 19th edition, 1995). The present invention is not limited by the route of administration.

Although an understanding of the mechanism is not necessary to practice the invention and the present invention is not limited to any particular mechanism of action, in some embodiments, mucosal vaccination is the preferred route of administration. In some embodiments, mucosal vaccination, such as intranasal vaccination, may induce mucosal immunity not only in the nasal mucosa, but also in distant mucosal sites such as the genital mucosa (See, e.g., Mestecky, Journal of Clinical Immunology, 7:265-276, 1987). More advantageously, in further preferred embodiments, in addition to inducing mucosal immune responses, mucosal vaccination also induces systemic immunity. In some embodiments, non-parenteral administration (e.g., muscosal administration of vaccines) provides an efficient and convenient way to boost systemic immunity (e.g., induced by parenteral or mucosal vaccination (e.g., in cases where multiple boosts are used to sustain a vigorous systemic immunity)).

In some embodiments, a composition comprising a nanoemulsion adjuvant and an immunogen of the invention may be used to protect or treat a subject susceptible to, or suffering from, disease and/or infection by means of administering a composition of the present invention via a mucosal route (e.g., an oral/alimentary or nasal route). Alternative mucosal routes include intravaginal and intra-rectal routes. In preferred embodiments of the present invention, a nasal route of administration is used, termed “intranasal administration” or “intranasal vaccination” herein. Methods of intranasal vaccination are well known in the art, including the administration of a droplet or spray form of the vaccine into the nasopharynx of a subject to be immunized. In some embodiments, a nebulized or aerosolized composition comprising a nanoemulsion adjuvant and immunogen is provided. Enteric formulations such as gastro resistant capsules for oral administration, suppositories for rectal or vaginal administration also form part of this invention.

Compositions of the present invention may also be administered via the oral route. Under these circumstances, a composition comprising a nanoemulsion adjuvant and an immunogen may comprise a pharmaceutically acceptable excipient and/or include alkaline buffers, or enteric capsules. Formulations for nasal delivery may include those with dextran or cyclodextran and saponin as an adjuvant.

Compositions of the present invention may also be administered via a vaginal route. In such cases, a composition comprising a nanoemulsion adjuvant and an immunogen may comprise pharmaceutically acceptable excipients and/or emulsifiers, polymers (e.g., CARBOPOL), and other known stabilizers of vaginal creams and suppositories. In some embodiments, compositions of the present invention are administered via a rectal route. In such cases, a composition comprising a NE and an immunogen may comprise excipients and/or waxes and polymers known in the art for forming rectal suppositories.

In some embodiments, the same route of administration (e.g., mucosal administration) is chosen for both a priming and boosting vaccination. In some embodiments, multiple routes of administration are utilized (e.g., at the same time, or, alternatively, sequentially) in order to stimulate an immune response (e.g., using a composition comprising a nanoemulsion adjuvant and immunogen of the present invention).

For example, in some embodiments, a composition comprising a nanoemulsion adjuvant and an immunogen is administered to a mucosal surface of a subject in either a priming or boosting vaccination regime. Alternatively, in some embodiments, a composition comprising a nanoemulsion adjuvant and an immunogen is administered systemically in either a priming or boosting vaccination regime. In some embodiments, a composition comprising a nanoemulsion adjuvant and an immunogen is administered to a subject in a priming vaccination regimen via mucosal administration and a boosting regimen via systemic administration. In some embodiments, a composition comprising a nanoemulsion adjuvant and an immunogen is administered to a subject in a priming vaccination regimen via systemic administration and a boosting regimen via mucosal administration. Examples of systemic routes of administration include, but are not limited to, a parenteral, intramuscular, intradermal, transdermal, subcutaneous, intraperitoneal or intravenous administration. A composition comprising a NE and an immunogen may be used for both prophylactic and therapeutic purposes.

In some embodiments, compositions of the present invention are administered by pulmonary delivery. For example, a composition of the present invention can be delivered to the lungs of a subject (e.g., a human) via inhalation (e.g., thereby traversing across the lung epithelial lining to the blood stream (See, e.g., Adjei, et al. Pharmaceutical Research 1990; 7:565-569; Adjei, et al. Int. J. Pharmaceutics 1990; 63:135-144; Braquet, et al. J. Cardiovascular Pharmacology 1989 143-146; Hubbard, et al. (1989) Annals of Internal Medicine, Vol. III, pp. 206-212; Smith, et al. J. Clin. Invest. 1989; 84:1145-1146; Oswein, et al. “Aerosolization of Proteins”, 1990; Proceedings of Symposium on Respiratory Drug Delivery II Keystone, Colorado; Debs, et al. J. Immunol. 1988; 140:3482-3488; and U.S. Pat. No. 5,284,656 to Platz, et al, each of which are hereby incorporated by reference in its entirety). A method and composition for pulmonary delivery of drugs for systemic effect is described in U.S. Pat. No. 5,451,569 to Wong, et al., hereby incorporated by reference; See also U.S. Pat. No. 6,651,655 to Licalsi et al., hereby incorporated by reference in its entirety)).

Further contemplated for use in the practice of this invention are a wide range of mechanical devices designed for pulmonary and/or nasal mucosal delivery of pharmaceutical agents including, but not limited to, nebulizers, metered dose inhalers, and powder inhalers, all of which are familiar to those skilled in the art. Some specific examples of commercially available devices suitable for the practice of this invention are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). All such devices require the use of formulations suitable for dispensing of the therapeutic agent. Typically, each formulation is specific to the type of device employed and may involve the use of an appropriate propellant material, in addition to the usual diluents, adjuvants, surfactants, carriers and/or other agents useful in therapy. Also, the use of liposomes, microcapsules or microspheres, inclusion complexes, or other types of carriers is contemplated.

Thus, in some embodiments, a composition comprising a nanoemulsion adjuvant of the present invention may be used to protect and/or treat a subject susceptible to, or suffering from, a disease or infection by means of administering a compositions comprising a nanoemulsion adjuvant by mucosal, intramuscular, intraperitoneal, intradermal, transdermal, pulmonary, intravenous, subcutaneous or other route of administration described herein. Methods of systemic administration of the adjuvant preparations may include conventional syringes and needles, or devices designed for ballistic delivery of solid vaccines (See, e.g., WO 99/27961, hereby incorporated by reference), or needleless pressure liquid jet device (See, e.g., U.S. Pat. No. 4,596,556; U.S. Pat. No. 5,993,412, each of which are hereby incorporated by reference), or transdermal patches (See, e.g., WO 97/48440; WO 98/28037, each of which are hereby incorporated by reference). The present invention may also be used to enhance the immunogenicity of antigens applied to the skin (transdermal or transcutaneous delivery, See, e.g., WO 98/20734; WO 98/28037, each of which are hereby incorporated by reference). Thus, in some embodiments, the present invention provides a delivery device for systemic administration, pre-filled with the adjuvant composition of the present invention.

The present invention is not limited by the type of subject administered (e.g., in order to stimulate an immune response (e.g., in order to generate protective immunity (e.g., mucosal and/or systemic immunity))) a composition of the present invention. Indeed, a wide variety of subjects are contemplated to be benefited from administration of a composition of the present invention. In preferred embodiments, the subject is a human. In some embodiments, human subjects are of any age (e.g., adults, children, infants, etc.) that have been or are likely to become exposed to a microorganism. In some embodiments, the human subjects are subjects that are more likely to receive a direct exposure to pathogenic microorganisms or that are more likely to display signs and symptoms of disease after exposure to a pathogen (e.g., immune suppressed subjects). In some embodiments, the general public is administered (e.g., vaccinated with) a composition of the present invention (e.g., to prevent the occurrence or spread of disease). For example, in some embodiments, compositions and methods of the present invention are utilized to vaccinate a group of people (e.g., a population of a region, city, state and/or country) for their own health (e.g., to prevent or treat disease). In some embodiments, the subjects are non-human mammals (e.g., pigs, cattle, goats, horses, sheep, or other livestock; or mice, rats, rabbits or other animal). In some embodiments, compositions and methods of the present invention are utilized in research settings (e.g., with research animals).

A composition of the present invention may be formulated for administration by any route, such as mucosal, oral, topical, parenteral or other route described herein. The compositions may be in any one or more different forms including, but not limited to, tablets, capsules, powders, granules, lozenges, foams, creams or liquid preparations.

Topical formulations of the present invention may be presented as, for instance, ointments, creams or lotions, foams, and aerosols, and may contain appropriate conventional additives such as preservatives, solvents (e.g., to assist penetration), and emollients in ointments and creams.

Topical formulations may also include agents that enhance penetration of the active ingredients through the skin. Exemplary agents include a binary combination of N-(hydroxyethyl) pyrrolidone and a cell-envelope disordering compound, a sugar ester in combination with a sulfoxide or phosphine oxide, and sucrose monooleate, decyl methyl sulfoxide, and alcohol.

Other exemplary materials that increase skin penetration include surfactants or wetting agents including, but not limited to, polyoxyethylene sorbitan mono-oleoate (Polysorbate 80); sorbitan mono-oleate (Span 80); p-isooctyl polyoxyethylene-phenol polymer (Triton WR-1330); polyoxyethylene sorbitan tri-oleate (Tween 85); dioctyl sodium sulfosuccinate; and sodium sarcosinate (Sarcosyl NL-97); and other pharmaceutically acceptable surfactants.

In certain embodiments of the invention, compositions may further comprise one or more alcohols, zinc-containing compounds, emollients, humectants, thickening and/or gelling agents, neutralizing agents, and surfactants. Water used in the formulations is preferably deionized water having a neutral pH. Additional additives in the topical formulations include, but are not limited to, silicone fluids, dyes, fragrances, pH adjusters, and vitamins.

Topical formulations may also contain compatible conventional carriers, such as cream or ointment bases and ethanol or oleyl alcohol for lotions. Such carriers may be present as from about 1% up to about 98% of the formulation. The ointment base can comprise one or more of petrolatum, mineral oil, ceresin, lanolin alcohol, panthenol, glycerin, bisabolol, cocoa butter and the like.

In some embodiments, pharmaceutical compositions of the present invention may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the nanoemulsion adjuvant and immunogen of the formulation. In some embodiments, immunostimulatory compositions of the present invention are administered in the form of a pharmaceutically acceptable salt. When used the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives may include benzalkonium chloride (0.003-0.03% w/v); chlorobutanol (0.3-0.9% w/v); parabens (0.01-0.25% w/v) and thimerosal (0.004-0.02% w/v).

In some embodiments, a composition comprising a nanoemulsion adjuvant is co-administered with one or more antibiotics. For example, one or more antibiotics may be administered with, before and/or after administration of a composition comprising a nanoemulsion adjuvant. The present invention is not limited by the type of antibiotic co-administered. Indeed, a variety of antibiotics may be co-administered including, but not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, doxycycline, quinolones (e.g., ciprofloxacin), sulfonamides, trimethoprim, and quinolines.

There are an enormous amount of antimicrobial agents currently available for use in treating bacterial, fungal and viral infections. For a comprehensive treatise on the general classes of such drugs and their mechanisms of action, the skilled artisan is referred to Goodman & Gilman's “The Pharmacological Basis of Therapeutics” Eds. Hardman et al., 9th Edition, Pub. McGraw Hill, chapters 43 through 50, 1996, (herein incorporated by reference in its entirety). Generally, these agents include agents that inhibit cell wall synthesis (e.g., penicillins, cephalosporins, cycloserine, vancomycin, bacitracin); and the imidazole antifungal agents (e.g., miconazole, ketoconazole and clotrimazole); agents that act directly to disrupt the cell membrane of the microorganism (e.g., detergents such as polmyxin and colistimethate and the antifungals nystatin and amphotericin B); agents that affect the ribosomal subunits to inhibit protein synthesis (e.g., chloramphenicol, the tetracyclines, erthromycin and clindamycin); agents that alter protein synthesis and lead to cell death (e.g., aminoglycosides); agents that affect nucleic acid metabolism (e.g., the rifamycins and the quinolones); the antimetabolites (e.g., trimethoprim and sulfonamides); and the nucleic acid analogues such as zidovudine, gangcyclovir, vidarabine, and acyclovir which act to inhibit viral enzymes essential for DNA synthesis. Various combinations of antimicrobials may be employed.

The present invention also includes methods involving co-administration of a composition comprising a nanoemulsion adjuvant with one or more additional active and/or immunostimulatory agents. Indeed, it is a further aspect of this invention to provide methods for enhancing prior art immunostimulatory methods (e.g., immunization methods) and/or pharmaceutical compositions by co-administering a composition of the present invention. In co-administration procedures, the agents may be administered concurrently or sequentially. In one embodiment, the compositions described herein are administered prior to the other active agent(s). The pharmaceutical formulations and modes of administration may be any of those described herein. In addition, the two or more co-administered agents may each be administered using different modes (e.g., routes) or different formulations. The additional agents to be co-administered (e.g., antibiotics, adjuvants, etc.) can be any of the well-known agents in the art, including, but not limited to, those that are currently in clinical use.

In some embodiments, a composition comprising a nanoemulsion adjuvant is administered to a subject via more than one route. For example, a subject that would benefit from having a protective immune response (e.g., immunity) towards a pathogenic microorganism may benefit from receiving mucosal administration (e.g., nasal administration or other mucosal routes described herein) and, additionally, receiving one or more other routes of administration (e.g., parenteral or pulmonary administration (e.g., via a nebulizer, inhaler, or other methods described herein). In some preferred embodiments, administration via mucosal route is sufficient to induce both mucosal as well as systemic immunity towards an immunogen or organism from which the immunogen is derived. In other embodiments, administration via multiple routes serves to provide both mucosal and systemic immunity. Thus, although an understanding of the mechanism is not necessary to practice the present invention and the present invention is not limited to any particular mechanism of action, in some embodiments, it is contemplated that a subject administered a composition of the present invention via multiple routes of administration (e.g., immunization (e.g., mucosal as well as airway or parenteral administration of a composition comprising a nanoemulsion adjuvant of the present invention) may have a stronger immune response to an immunogen than a subject administered a composition via just one route.

Other delivery systems can include time-release, delayed release or sustained release delivery systems. Such systems can avoid repeated administrations of the compositions, increasing convenience to the subject and a physician. Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109, hereby incorporated by reference. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, each of which is hereby incorporated by reference and (b) diffusional systems in which an active component permeates at a controlled rate from a polymer such as described in U.S. Pat. Nos. 3,854,480, 5,133,974 and 5,407,686, each of which is hereby incorporated by reference. In addition, pump-based hardware delivery systems can be used, some of which are adapted for implantation.

In preferred embodiments, a composition comprising a nanoemulsion adjuvant and an immunogen of the present invention comprises a suitable amount of the immunogen to induce an immune response in a subject when administered to the subject. In preferred embodiments, the immune response is sufficient to provide the subject protection (e.g., immune protection) against a subsequent exposure to the immunogen or the microorganism (e.g., bacteria or virus) from which the immunogen was derived. The present invention is not limited by the amount of immunogen used. In some preferred embodiments, the amount of immunogen (e.g., virus or bacteria neutralized by the nanoemulsion adjuvant, or, recombinant protein) in a composition comprising a nanoemulsion adjuvant and immunogen (e.g., for use as an immunization dose) is selected as that amount which induces an immunoprotective response without significant, adverse side effects. The amount will vary depending upon which specific immunogen or combination thereof is/are employed, and can vary from subject to subject, depending on a number of factors including, but not limited to, the species, age and general condition (e.g., health) of the subject, and the mode of administration. Procedures for determining the appropriate amount of immunogen administered to a subject to elicit an immune response (e.g., a protective immune response (e.g., protective immunity)) in a subject are well known to those skilled in the art.

In some embodiments, it is expected that each dose (e.g., of a composition comprising a nanoemulsion adjuvant and an immunogen (e.g., administered to a subject to induce an immune response (e.g., a protective immune response (e.g., protective immunity))) comprises 0.05-5000 μg of each immunogen (e.g., recombinant and/or purified protein), in some embodiments, each dose will comprise 1-500 μg, in some embodiments, each dose will comprise 350-750 μg, in some embodiments, each dose will comprise 50-200 μg, in some embodiments, each dose will comprise 25-75 μg of immunogen (e.g., recombinant and/or purified protein). In some embodiments, each dose comprises an amount of the immunogen sufficient to generate an immune response. An effective amount of the immunogen in a dose need not be quantified, as long as the amount of immunogen generates an immune response in a subject when administered to the subject. An optimal amount for a particular administration (e.g., to induce an immune response (e.g., a protective immune response (e.g., protective immunity))) can be ascertained by one of skill in the art using standard studies involving observation of antibody titers and other responses in subjects.

In some embodiments, it is expected that each dose (e.g., of a composition comprising a nanoemulsion adjuvant and an immunogen (e.g., administered to a subject to induce and immune response)) is from 0.001 to 15% or more (e.g., 0.001-10%, 0.5-5%, 1-3%, 2%, 6%, 10%, 15% or more) by weight immunogen (e.g., neutralized bacteria or virus, or recombinant and/or purified protein). In some embodiments, an initial or prime administration dose contains more immunogen than a subsequent boost dose

In some embodiments, a composition comprising a nanoemulsion adjuvant of the present invention is formulated in a concentrated dose that can be diluted prior to administration to a subject. For example, dilutions of a concentrated composition may be administered to a subject such that the subject receives any one or more of the specific dosages provided herein. In some embodiments, dilution of a concentrated composition may be made such that a subject is administered (e.g., in a single dose) a composition comprising about 0.1-50% of the nanoemulsion adjuvant present in the concentrated composition. In some preferred embodiments, a subject is administered in a single dose a composition comprising 1% of the NE and immunogen present in the concentrated composition. Concentrated compositions are contemplated to be useful in a setting in which large numbers of subjects may be administered a composition of the present invention (e.g., an immunization clinic, hospital, school, etc.). In some embodiments, a composition comprising a nanoemulsion adjuvant of the present invention (e.g., a concentrated composition) is stable at room temperature for more than 1 week, in some embodiments for more than 2 weeks, in some embodiments for more than 3 weeks, in some embodiments for more than 4 weeks, in some embodiments for more than 5 weeks, and in some embodiments for more than 6 weeks.

Generally, the emulsion compositions of the invention will comprise at least 0.001% to 100%, preferably 0.01 to 90%, of emulsion per ml of liquid composition. It is envisioned that the formulations may comprise about 0.001%, about 0.0025%, about 0.005%, about 0.0075%, about 0.01%, about 0.025%, about 0.05%, about 0.075%, about 0.1%, about 0.25%, about 0.5%, about 1.0%, about 2.5%, about 5%, about 7.5%, about 10%, about 12.5%, about 15%, about 20%, about 25%, about 30%, about 35%, about 40%, about 50%, about 55%, about 60%, about 65%, about 70%, about 75%, about 80%, about 85%, about 90%, about 95% or about 100% of emulsion per ml of liquid composition. It should be understood that a range between any two figures listed above is specifically contemplated to be encompassed within the metes and bounds of the present invention. Some variation in dosage will necessarily occur depending on the condition of the specific pathogen and the subject being immunized.

In some embodiments, following an initial administration of a composition of the present invention (e.g., an initial vaccination), a subject may receive one or more boost administrations (e.g., around 2 weeks, around 3 weeks, around 4 weeks, around 5 weeks, around 6 weeks, around 7 weeks, around 8 weeks, around 10 weeks, around 3 months, around 4 months, around 6 months, around 9 months, around 1 year, around 2 years, around 3 years, around 5 years, around 10 years) subsequent to a first, second, third, fourth, fifth, sixth, seventh, eighth, ninth, tenth, and/or more than tenth administration. Although an understanding of the mechanism is not necessary to practice the present invention and the present invention is not limited to any particular mechanism of action, in some embodiments, reintroduction of an immunogen in a boost dose enables vigorous systemic immunity in a subject. The boost can be with the same formulation given for the primary immune response, or can be with a different formulation that contains the immunogen. The dosage regimen will also, at least in part, be determined by the need of the subject and be dependent on the judgment of a practitioner.

Dosage units may be proportionately increased or decreased based on several factors including, but not limited to, the weight, age, and health status of the subject. In addition, dosage units may be increased or decreased for subsequent administrations (e.g., boost administrations).

A composition comprising an immunogen of the present invention finds use where the nature of the infectious and/or disease causing agent (e.g., for which protective immunity is sought to be elicited) is known, as well as where the nature of the infectious and/or disease causing agent is unknown (e.g., in emerging disease (e.g., of pandemic proportion (e.g., influenza or other outbreaks of disease))). For example, the present invention contemplates use of the compositions of the present invention in treatment of or prevention of infections associated with an emergent infectious and/or disease causing agent yet to be identified (e.g., isolated and/or cultured from a diseased person but without genetic, biochemical or other characterization of the infectious and/or disease causing agent).

It is contemplated that the compositions and methods of the present invention will find use in various settings, including research settings. For example, compositions and methods of the present invention also find use in studies of the immune system (e.g., characterization of adaptive immune responses (e.g., protective immune responses (e.g., mucosal or systemic immunity))). Uses of the compositions and methods provided by the present invention encompass human and non-human subjects and samples from those subjects, and also encompass research applications using these subjects. Compositions and methods of the present invention are also useful in studying and optimizing nanoemulsions, immunogens, and other components and for screening for new components. Thus, it is not intended that the present invention be limited to any particular subject and/or application setting.

The formulations can be tested in vivo in a number of animal models developed for the study of mucosal and other routes of delivery. As is readily apparent, the compositions of the present invention are useful for preventing and/or treating a wide variety of diseases and infections caused by viruses, bacteria, parasites, and fungi, as well as for eliciting an immune response against a variety of antigens. Not only can the compositions be used prophylactically or therapeutically, as described above, the compositions can also be used in order to prepare antibodies, both polyclonal and monoclonal (e.g., for diagnostic purposes), as well as for immunopurification of an antigen of interest. If polyclonal antibodies are desired, a selected mammal, (e.g., mouse, rabbit, goat, horse, etc.) can be immunized with the compositions of the present invention. The animal is usually boosted 2-6 weeks later with one or more—administrations of the antigen. Polyclonal antisera can then be obtained from the immunized animal and used according to known procedures (See, e.g., Jurgens et al., J. Chrom. 1985, 348:363-370).

In some embodiments, the present invention provides a kit comprising a composition comprising a nanoemulsion adjuvant. In some embodiments, the kit further provides a device for administering the composition. The present invention is not limited by the type of device included in the kit. In some embodiments, the device is configured for nasal application of the composition of the present invention (e.g., a nasal applicator (e.g., a syringe) or nasal inhaler or nasal mister). In some embodiments, a kit comprises a composition comprising a nanoemulsion adjuvant in a concentrated form (e.g., that can be diluted prior to administration to a subject).

In some embodiments, all kit components are present within a single container (e.g., vial or tube). In some embodiments, each kit component is located in a single container (e.g., vial or tube). In some embodiments, one or more kit component are located in a single container (e.g., vial or tube) with other components of the same kit being located in a separate container (e.g., vial or tube). In some embodiments, a kit comprises a buffer. In some embodiments, the kit further comprises instructions for use.

### EXAMPLES

The following examples serve to illustrate certain preferred embodiments and aspects of the present invention and are not to be construed as limiting the scope thereof.

In the experimental disclosure which follows, the following abbreviations apply: eq (equivalents); μ (micron); M (Molar); μM (micromolar); mM (millimolar); N (Normal); mol (moles); mmol (millimoles); pmol (micromoles); nmol (nanomoles); g (grams); mg (milligrams); μg (micrograms); ng (nanograms); L (liters); ml (milliliters); μl (microliters); cm (centimeters); mm (millimeters); μm (micrometers); nM (nanomolar); ° C. (degrees Centigrade); and PBS (phosphate buffered saline).

### Example 1

### Materials and methods

Mice. Naïve 8-10 week-old female CD-1 and C57BL/6N mice were purchased from Charles River Laboratories (Wilmington, Mass.). Naïve 8-10 week-old female B6.Cg-Tg(HLA-A/H2-D)2Enge/J, IL-6 gene deficient mice (IL-6−/−) [B6.129s2-IL6^{TM1KOPF}/J] and C57BL/6J mice were purchased from Jackson Laboratories (Bar Harbor, Me.). All mice were housed in specific pathogen-free conditions in facilities maintained by the University of Michigan Unit for Laboratory Animal Medicine. The University Committee on Use and Care of Animals (UCUCA) at the University of Michigan approved all procedures performed on mice.

Cell lines. Primary nasal epithelial cells were cultured from the nasal septum of C57BL/6N mice as described in Antunes et al., Biotechniques 2007. 43: 195-196, 198, 200 passim). Mouse bone marrow derived dendritic cells (BMDC) were generated (See Lutz et al., J. Immunol. Methods 1999. 223: 77-92). Briefly, BMDCs were isolated from C57BL/6 mouse femurs and tibias. On day 0, BMDCs were re-suspended in RPMI 1640 medium with L-glutamine (MEDIATECH, Inc., Manassas, Va.) supplemented with 5% heat-inactivated FBS (Gemini), 100 U/ml of penicillin-100 μg/ml of streptomycin (MEDIATECH, Inc), 1 mM sodium pyruvate (GIBCO, Carlsbad, Calif.), 100 μM MEM NEAA (Gibco), 50 μM 2-mercaptoethanol (Sigma-Aldrich, St. Louis, Mo.) and 3 ng/ml mGM-CSF (R&D Systems, Emeryville, Calif.) and cultured in T-75 tissue culture flasks (Corning, Union City, Calif.) for 4 days. On day 4 the immature DCs were collected using gentle scraping, re-suspended at 1.33×10⁶ cells/ml and plated at 3 ml/well (4×10⁶ cells/well) in 6-well tissue culture plates (Corning-Costar) overnight.

The murine pulmonary epithelial cell line TC-1 was obtained from ATCC(CRL-2785) and cultured in RPMI 1640 containing L-glutamine (2 mM) supplemented with 10 mM HEPES, 1 mM sodium pyruvate, 100 μM MEM NEAA, 10% heat-inactivated FBS, 100 μg/ml Penicillin, and 100 μg/ml Streptomycin.

Nanoemulsions. Nanoemulsion were obtained from NANOBIO Corporation (Ann Arbor, Mich.) and obtained by nanoemulsification of Tween 80 (or poloxamer 407), cetylpyridinium chloride (CPC), ethanol as a solvent, highly purified soybean oil, and water (referred to herein as W₈₀5EC (NE) or P₄₀₇5EC (NEₚ). Nonionic nanoemulsions were manufactured by emulsifying without CPC (referred to herein as W₈₀5E).

Antigens. Enhanced green fluorescent protein (GFP) was acquired from BioVision Research Products (San Francisco, Calif.). ENDOGRADE ovalbumin (OVA) was purchased from Hyglos (Am Neuland, Germany). Alexa Fluor 647-conjugated OVA protein (OVA-Alexa 647) were purchased from INVITROGEN. Recombinant rPA from B. anthracis were purchased from List Biological Laboratories, Inc (Campbell, Calif.). QDOTs (Qtracker 655 non-targeted) were purchased from Quantum Dot Corporation (Hayward, Calif.). Prolong Gold and DAPI were purchased from MOLECULAR PROBES (Carlsbad, Calif.).

Immunization protocol. Groups of B6.Cg-Tg(HLA-A/H2-D)2Enge/J mice were immunized on day 0 intranasally (i.n.) with 20 μg HBsAg (15 μl/mouse), 1 μg cholera toxin (CT-Sigma-Aldrich)+20 μg HBsAg (15 μl/mouse), or 20% NE+20 μg HBsAg (15 μl/mouse). Mice were boosted with their respective vaccines on days 28 and 56. The negative control group received 15 μl of sterile PBS at the same time points. The mice were phlebotomized every 14 days and serum IgG titers were determined by an antigen-specific ELISA (See Makidon et al., PLoS ONE 2008. 3: e2954). Animals were euthanized 2 weeks following the last boost and a post-mortem bronchial lavage was collected and analyzed for secreted anti-HBsAg IgA (See Makidon et al., PLoS ONE 2008. 3: e2954).

For studies evaluating the role of IL-6, groups of IL-6−/− and WT (C57BL/6J) mice were i. n. immunized with 20 μg rPA±20% NE (12 μl), or sterile PBS (negative controls) on days 0 and 28. At week 6, the animals were euthanized and the spleens were immediately harvested and processed into a single cell suspension. rPA-specific in vitro recall responses were analyzed as described below (See Bielinska et al., Infect Immun 2007. 75: 4020-4029).

Enzyme-linked immunosorbent assay (ELISA). Antigen-specific IgG and IgA responses were measured by ELISA with 5 μg/ml of HBsAg or rPA (See Makidon et al., PLoS ONE 2008. 3: e2954 and Bielinska et al., Infect Immun 2007. 75: 4020-4029). Anti-mouse IgG and IgA-alkaline phosphatase conjugated antibodies were from Jackson ImmunoResearch Laboratories Inc. (West Grove, Pa.). Alkaline phosphatase (AP) conjugated rabbit anti-mouse IgG (H&L) and IgA (a chain specific) antibodies were purchased from Rockland Immunochemicals, Inc. (Gilbertsville, Pa.).

Multiplex Cytokine & Chemokine Immunoassay. Spleens were harvested from immunized mice directly following euthanasia and processed for in vitro OVA-specific T-cell cytokine response (See Makidon et al., PLoS ONE 2008. 3: e2954). In brief, a single-cell suspension of 4×10⁶ cells/ml of splenocytes was incubated with 5 μg/ml of either OVA or rPA for 72 hours. Supernatant was evaluated for antigen-specific cytokine/chemokines using Multiplex Mouse Cytokine Chemokine Immunoassay Multiplex22 (Millipore Corp., Billerica, Mass.) according to manufacturer's instructions. Cytokine concentrations were calculated based on standard curve data using a MASTERPLEX QT Analysis version 2 (MiraiBio).

Evaluation of presence of CPC in olfactory tissues. The presence of CPC was evaluated in brain tissues in 10 week-old C57BL/6N mice treated nasally with 10 μg of CT+30 μg CPC (the equivalent concentration of CPC in 15 μl of 20% NE) (15 μl), 20% NE (15 μl), or sterile PBS (15 μl). CPC residues in olfactory tissues were determined using a high-performance liquid chromatography assay. For analysis, whole brain samples were mechanically disrupted with a spatula and extracted with 1 ml of 95% ethanol at 60° C. for 1 hr with simultaneous sonication. Samples were analyzed using Waters Symmetry C-18, 5 μm, 150×3.9 mm and a UV-detector set at 260 nm. An ethanol blank was injected to show free from interference. Samples were reported LOQ/LOD of the method 0.01 μl/ml. These samples were also normalized to tissue wet weight.

Quantum dots assay in vivo. Quantum dots were used as a model antigen due to their high level of in vivo fluorescence and their ability to interact with the lymphoid tissues in the mice (See Ballou et al. 2007. 18: 389-396.). Groups of 4 mice were inoculated with 15 μL of QDOTs (6.6 μL of 2 μM solution, ±20% NE) using a pipette. In-life fluorescence analysis was performed in isoflurane-anesthetized mice using the IVIS Imaging System 200 Spectrum series bioluminometer (Xenogen) in the Center for Molecular Imaging at the University of Michigan. The fluorescent measurement was quantified using IVIS Living Image 3.1 software. These experiments were performed twice with comparable results.

Antigen localization. To determine the localization of GFP antigen in different tissues 18 hours after nasal vaccination, the mice were nasally immunized (15 μL/mouse) using a pipette with a mixture of GFP (10 μg/mouse, green fluorescence)±NE (20%). The animals were sacrificed 18 hours following inoculation, and nasal epithelium, superficial cervical lymph nodes, mediastinal lymph nodes, and organized nasal associated lymphoid tissues (NALT) (See Asanuma et al., J Immunol Methods 1997. 202: 123-131) tissues were collected in OTC (TissueTek, Sakura Finetek, Torrance, Calif.) and frozen by slow immersion in liquid nitrogen. Tissue sections were cut in 5 μm sections and placed on glass slides for evaluation. Nasal epithelium, superficial cervical lymph node, and mediastinal lymph node tissues were analyzed using a Leica DCS 480 epifluoreescent micropscope and LAS vs. 3.10 software (Leica, Wetzlar, Germany). NALT tissues were imaged with a Zeiss LSM501 laser confocal microscopy using HeNel, Argon, and Enterprise lasers. These experiments were performed twice with comparable results.

FACS analysis. Immunofluorescent studies of NALT tissues were confirmed using FACS. The presence of OVA-Alexa 647 was analyzed in single cell suspensions derived from NALT tissues of CD-1 mice 36 hours flowing nasal immunization with 10 μg OVA-Alexa 647±20% NE (15 μl/mouse). The cells were stained with APC hamster anti-mouse cD11c (BD PHARMINGEN, Sparks, Md.), rat anti-mouse cD19: PACIFIC BLUE (AbD serotec, Raleigh, N.C.), PE anti-mouse cD11b (BioLegend) or their respective isotype controls including APC hamster IgG1, λ1 (BD PHARMINGEN), rat IgG2a: pacific Blue (AbD serotec) or PE rat IgG22b κ (BioLegend, San Diego, Calif.). Samples were acquired using a LSR II (BD Biosciences). Data was analyzed on DIVA software (BD Biosciences).

FACS surface identification of leukocytes infiltrating the nasal epithelium following NE-based immunization. To analyze the presence of infiltrating leukocytes at the site of immunization, the nasal septum was harvested from mice 18 hours following nasal treatment with 20% NE (15 μl), 20 μg OVA in PBS (15 μl), 20% NE+20 μg OVA (15 μl), or PBS (15 μl). The septal tissue was processed into a single cell suspension and suspended in FACS buffer (Biolegend) and blocked on ice using purified anti-mouse CD16/CD32 (Biolegend 101302). The cells were then stained with AlexaFluor 647 CD11b antibody (Biolegend 101218), AlexaFluor 647 CD11c antibody (Biolegend 117312), AlexaFluor 647 CD205 DEC-205 antibody (Biolegend 138204), AlexaFluor 647 CD45R/B220 antibody (Biolegend 103226), rat anti mouse Gr-1 AlexaFluor 488 (AbD MCA2387A488), and/or rat anti mouse F4/80 AlexaFluor 647 (AbD MCA497A647) for 20 minutes on ice. The results were compared to cells stained with the appropriate isotype controls. The cells were then washed twice and resuspended in FACS buffer prior to analysis via flow cytometer using a BD Accuri C6 Flow cytometer (Accuri, Ann Arbor, Mich.).

FACS analysis of NE-inducible MHC class II expression in primary nasal epithelial cells. A primary culture of nasal septal epithelial cells was prepared as above. 1×10⁶ cells were incubated with 0.001%, or 0.01% or 0.1% NE. Positive controls included 100 ng-500 ng LPS (E. coli K12) or 400 units of recombinant mouse TNF-α (Invitrogen, Carlsbad, Calif.). Negative controls included no treatment (media alone). The treated cells were washed, suspended in FACS buffer (Biolegend) and blocked on ice with TruStain fcX, Biolegend). The cells were then stained with 0.25 μg/10⁶ cells of anti-1-A/1-E-alexa flour 647 (Biolegend) or isotype control for 20 minutes on ice. The cells were then washed twice and resuspended in FACS buffer prior to analysis via flow cytometer using a BD Accuri C6 Flow cytometer.

Phenotype identification of NE-mediated APCs trafficking to draining lymph nodes. Superficial and deep cervical lymph nodes were harvested directly post-mortem from CD-1 mice 18 hours following treatment with GFP plus 20% NE (15 μl). The tissue was fixed in 10% buffered formalin and parrifinized. 5 μm tissue section slides were prepared in the Center for Organogenesis Morphology Core at the University of Michigan. The tissue sections were blocked for 10 minutes using the POWERBLOCK solution (BioGenex), and stained with anti-GFP fluorescent antibodies or control same isotype antibodies in PBS containing 0.1% BSA overnight at 4° C. Tissue was stained with the DC markers DEC205 (Rabbit anti-mouse CD205 mAb (Serotech)) and CD11c rabbit anti-mouse cD11b (Abcam)) or macorphage marker cD11b. GFP was detected with mAb anti-GFP (rabbit pAb anti-GFP (Biosystems)). Goat anti-rabbit DYLIGHT 594 (Biocare Medical) was used as a secondary antibody to rabbit primaries. The stained tissue samples were mounted in ProLong (DAPI blue nuclear stain). Imaging was performed using a Zeiss LSM501 laser confocal microscopy using HeNel, Argon, and Enterprise lasers.

Electron microscopy of nasal epithelium. The sinus cavities were excised 18 hours post-inoculation and immersion-fixed in 2.5% glutaraldehyde in 0.05 M cacodylate buffer, pH 7.4, at room temperature for 4 hours. After fixation, the sections were demineralized in 7.5 percent disodium EDTA with 2.5% glutaraldehyde for seven days following a protocol adapted from Shapiro et al. (See Shapiro et al., Anat Rec 1995. 241: 39-48. After demineralization, preparations were rinsed in cacodylate buffer, and then post-fixed for 1.5 hours in 1% osmium tetroxide in buffer. Next, they were dehydrated in an ascending graded series of ethanol and then transferred into three 30 minutes changes of propylene oxide. Then the nasal tissues were infiltrated and embedded in Epon. Ultra-thin sections were viewed without post-staining on a Philips CM100 at 60 kv. The images were recorded digitally using a Hamamatsu ORCA-HR digital camera system, which was operated using AMT software (Advanced Microscopy Techniques Corp., Woburn, Mass.) in the Microscopy and Image Laboratory at the University of Michigan.

Detection of apoptosis and necrosis. To evaluate the induction of NE-driven apoptosis of nasal epithelial cells, C57BL6/N mice were nasally treated with 20% NE (15 μl). To determine if the NE mixture itself or its components induce apoptosis of nasal epithelial cells, other mice were treated with either the equivalent concentration of CPC contained in 15 μl CPC (15 μl), with W805E (a non-ionic nanoemulsion) (15 μl), or with sterile PBS (15 μl). Nasal septal epithelium was harvested (See Antunes et al., Biotechniques 2007. 43: 195-196, 198, 200 passim) and fixed in 10% buffered formalin for 24 hours and parrafinized prior to microtome sectioning and de-parrifinization. Tissue processing and immunohistochemical staining was performed using an INTELLIPATH FLX (Biocare Medical, Concord, Calif.). Antigen retrieval was performed in DIVA decloacker buffer and blocked with peroxidase and Rodent block M (Biocare Medical) for 5 and 30 minutes respectively according to manufacturer's recommendations. Tissues were stained with a 1:1000 dilution of pAb rabbit anti-Caspase-3 pAb (Cell Signaling, Beverly, Mass.) or with a 1:3000 dilution of rabbit pAb anti-calreticulin (Abcam, Cambridge, Mass.) for 1 hr. Following wash, Rabbit-on-Rodent HRP-Polymer was applied for 30 minutes according to manufacturer guidelines. DAB chromogen was applied and the side was counterstained with Cat hematoxylin (Biocare medical). Stained tissues were imaged on an Olympus BX51 microscope.

Identification of cells dying by necrosis was characterized morphologically according to defined criteria (See Ziegler and Groscurth, News Physiol Sci 2004. 19: 124-128). In brief, necrotic cells were identified as cells containing dilated organelles and dissociated ribosomes from the endoplasmic reticulum. These cells do not contain pyknotic or fragmented nuclei and the degeneration proceeds without any detectable involvement of lysosomes.

Epithelial gene expression analysis using microarrays. To evaluate regulation of NE-mediated changes in gene expression in nasal epithelial tissues, nasal septal epithelium was harvested immediately post-mortem from CD-1 mice at either 6 hours or 24 hours following nasal treatment with either 20% NE (15 μl) or sterile PBS (15 μl). The tissue was collected in OTC and frozen by slow immersion in liquid nitrogen and stored at −80° C. until used for microarray analysis. Total RNA was extracted per sample using RNeasy (Qiagen) according to the manufacturer's instructions. RNA samples were pooled and processed by the UMCCC Affymetrix Core Facility at the University of Michigan using an Ovation Biotin Labeling system from NuGen, Inc. following manufacturer's protocols. Prior to hybridization, the quality of RNA was accessed using an Agilent 2100 Bioanalyzer following protocols established at UMCCC Affymetrix Core. Hybridization, detection and scanning was performed using a mouse GENECHIP 430 2.0 manufactured by Affymetrix and a Affymetrix Scanner 3000 following manufactures guidelines. Gene expression values were calculated using a robust multi-array average (RMA) (See Irizarry et al., Stat Appl Genet Mol Biol 2003. 2: Article1). Complete microarray data has been deposited in the public database Gene Expression Omnibus (GEO) under series accession number GSE25486.

Analysis of cytokine and chemokine expression in BMDC or in nasal septal tissues. BMDC were cultured for 5 days as described above. 4×10⁶ BMDCs/treatment were stimulated with 0.001%, 0.01% or 0.1% NE, 1 ug/ml, 10 ug/ml or 30 ug/ml Cholera Toxin (CT) (List Laboratories, Campbell, Calif.). As a positive control BMDCs were treated also with 1 ng/ml or 10 ng/ml LPS S. minnesota (List Laboratories) or left untreated as negative control. The cells were stimulated in 2 ml/well of the medium with lowered content of FBS (2%) and mGM-CSF (1.5 ng/ml) at 37° C. 5% CO₂ atmosphere for 24 hours. Cytokine secretion was measured in supernatant using bead-based multiplex assay according to manufacturer protocol as described above (Millipore Multiplex22).

Mucosal cytokines/chemokines were also analyzed in vivo. C57BL6/N mice were intranasally treated with 15 μL of 20% NE, 1 μg CT, or sterile PBS. Nasal septal epithelium was collected as above directly post-mortem 18 hours following treatment. The epithelium was manually homogenized using mortar and pestle and then gently digested using T-PER tissue extraction reagent (Thermo Scientific, Rockford, Ill.) according to manufacturer's recommendation. Cytokine secretion was measured in supernatant using bead-based multiplex assay according to manufacturer protocol as described above (Millipore Multiplex22). Additionally, the supernatant was evaluated for the presence of TGF-131 and thymic stromal lymphopoietin (TLSP) via ELISA using a mouse/rat/porcine/canine TGF-131 immunoassay kit (QUANTIKINE, R&D Systems, Minneapolis, Minn.) and Mouse TSLP Immunoassay kit (QUANTIKINE) according to manufacturer's recommendations.

To evaluate the potential for NE to mediate cytokine expression in epithelial cells, 4×10⁶ TC-1 cells were incubated with 0.001%, 0.01% or 0.1% NE. The supernatant was evaluated using ELISA for TGF-131, and TSLP as above. IL-6 was also measured using a custom ELISA. In brief, 96 well MAXISORP (Nunc, Rochester, N.Y.) plates were coated with 2 μg/ml rabbit pAB anti-IL-6 (Abcam, Cambridge, Mass.) and blocked with peroxidase. After washing, 100 μl of non-diluted supernatant/well was incubated on the plate for 2 hours. After washing, 50 μl of a 1:200 dilution biotinylated anti-IL6 antibody (Abcam) was incubated in each well for 2 hours. After washing, the plate was developed with streptavidin-HRP and read at an absorbance of 450 nm.

Statistical analysis. Statistical comparisons were assessed by Two-way ANOVA with TUKEY comparison, Student's t-test and Mann-Whitney test by using GraphPad Prism version 5.00, GraphPad Software (San Diego Calif.; www.graphpad.com). A p value<0.05 value was considered significant.

### Example 2

### Adjuvant Activity of Nanoemulsion Compared to Adjuvant Activity of Cholera Toxin

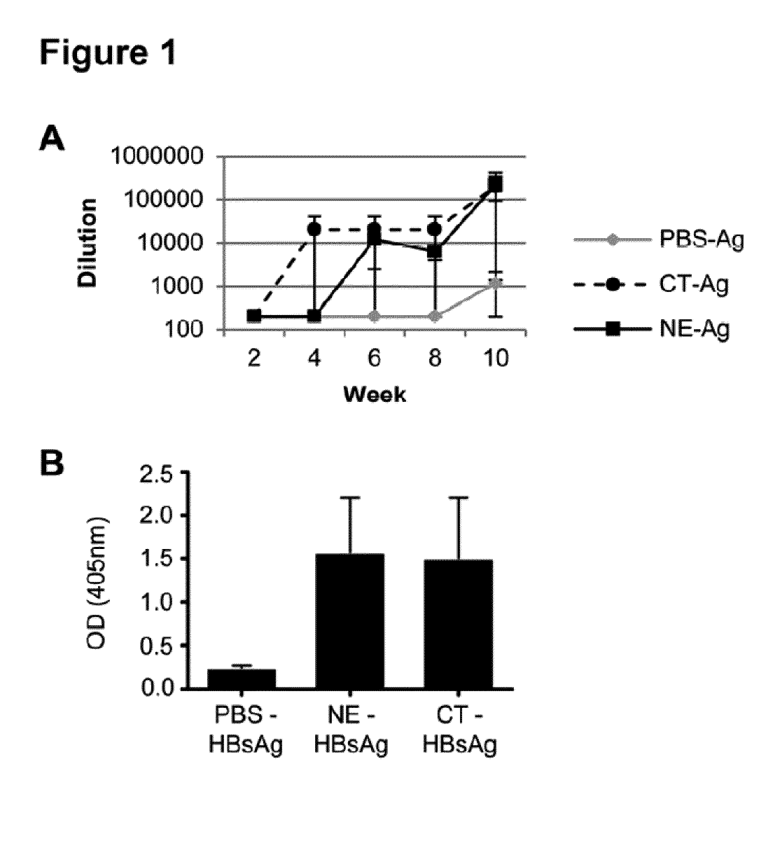

Nanoemulsion mucosal adjuvant ability to augment immune responses when co-administered with protein antigens was examined, and the adjuvant activities compared with activities achieved with cholera toxin (CT). Adult, specific pathogen free, female B6.Cg-Tg(HLA-A/H2-D)2Enge/J mice were nasally immunized with either 20% poloxamer 407-based nanoemulsion (NEₚ)+20 μg HBsAg, 1 μg CT+20 μg HBsAg, or 20 μg HBsAg alone 3 times four weeks apart (See FIG. 1A). The kinetics of the anti-HBsAg IgG serum antibody response was not significantly different (p>0.05) between the NE and CT immunized groups at any time point although the CT appeared to have a higher titer at 4 weeks prior to the boost. The end titer of anti-HBsAg IgG measured 6.4×10⁵ in CT-immunized mice compared to an end titer of anti-HBsAg IgG measuring 2.1×10⁵ in NE immunized mice. Measurement of secreted anti-HBsAg IgA revealed that both CT and NE stimulated equivalent local mucosal immune responses (p>0.05) (See FIG. 1B). Accordingly, in some embodiments, the invention provides that NE produces local and systemic immune responses after intranasal immunization with the NE and protein antigen (e.g., recombinant protein antigen (e.g., HBsAg)) that is highly similar to the local and systemic immune responses attained after intranasal immunization with a protein antigen and CT.

### Example 3

### Nanoemulsion Administration does not Cause Inflammation or Redirect Antigen to Olfactory Tissues

Adjuvant side effects are a significant concern and a barrier to human use. Therefore, studies were performed during development of embodiments of the invention in order to evaluate whether nanoemulsion produced any CNS inflammation, or whether nanoemulsion redirects antigen to olfactory tissues after intranasal immunization as has been reported with CT (See, e.g., van Ginkel et al., Infect Immun 2005. 73: 6892-6902; Couch, R. B., N Engl J Med 2004. 350: 860-861). A sensitive HPLC assay was used to characterize the presence of cetylpyridinium chloride (CPC), a component of the NE, in brain tissue from immunized animals. Whole brain samples were evaluated from C57B1/6N mice treated intranasally with 15 μl of 20% NE. The results were compared to measurements in tissues from mice intranasally administered 15 μl of either sterile PBS or 1 μg CT plus an equivalent concentration of CPC to that contained in 20% NE. CPC was not detectable in the olfactory tissues of PBS and 20% NE treated mice; however in CT-CPC treated mice, CPC was noted in brain tissues of 2/6 mice after co-administration with a minimal concentration of CT (1 μg). This result confirmed results from a Good Laboratory Practices (GLP) toxicity study conducted in rabbits, where neither olfactory bulb antigen localization nor inflammation was evident after nasal administration of a NE-based influenza vaccine. Thus, the invention provides, in some embodiments, that administration of nanoemulsion adjuvant to a subject (e.g., administration of 20% nanoemulsion adjuvant) does not redirect antigen to the CNS or cause brain inflammation, in sharp contrast to CT, which has been documented to cause both inflammation as well as redirection of antigen to the CNS (See, e.g., van Ginkel et al., Infect Immun 2005. 73: 6892-6902; Couch, R. B., N Engl J Med 2004. 350: 860-861). as has been reported with cholera toxin.

### Example 4

### Nanoemulsion Promotes Mucosal Antigen Uptake and Trafficking to Regional Draining Lymph Nodes In Vivo

Experiments were conducted during development of embodiments of the invention in order to determine whether NE mucosal adjuvant was capable of enhancing antigen uptake, cytokine production and activation of dendritic cell (DC) trafficking to regional lymph nodes. Prior to the generation of embodiments of the invention, that art has documented and has accepted as conventional practice that adjuvant toxicity (e.g., represented by associated inflammation, necrosis, etc.) plays a role in antigen uptake and cytokine production in local mucosa. For example, the art has documented that introduction of mutations into CT that eliminate ADP-ribosyltransferase mediated toxicity and that reduce induction of cAMP in cells leads to a decreased adjuvant activity of CT. Thus, prior to development of embodiments of the invention, it has been conventional wisdom in the art that an effective adjuvant must possess toxicity characteristics (e.g., represented by associated inflammatory response when administered to a subject), and it remained unknown whether a non-inflammatory material could augment mucosal immunogenicity.

Experiments were conducted in order to evaluate NE's capacity to enhance nasal mucosa DC antigen acquisition and subsequent migration of DC to cervical lymph nodes in vivo. For these studies, quantum dots (QDOTs) were nasally administered in the presence or absence of 20% NE. The distribution of QDOT-specific fluorescence was evaluated in live outbred CD-1 mice by imaging at 18 hours following nasal inoculation (See FIG. 2A). Significantly more fluorescence was observed at this time point in the nose (p=0.0049), cervical LN (p=2.24×10⁻⁴), and mediastinal LN (p=6.19×10⁻⁵) following nasal administration of NE-QDOTs as compared to QDOTs administered in PBS. Accordingly, in some embodiments, the invention provides enhanced uptake of antigen (e.g., QD) in regional lymphatic tissues after administration in NE compared to administration of antigen in the absence of NE.

In order to further characterize the whole body distribution of protein antigens after nasal administration in NE, 10 μg of GFP (used as a model protein antigen) was administered intranasally to CD-1 mice in the presence of 20% NE. As shown in FIG. 2B, 18 hours following nasal exposure, more fluorescence was detected throughout the nasal epithelium (bottom left), cervical LN (bottom middle panel) and mediastinal LN (bottom right panel) in GFP-NE treated mice than in mice exposed to GFP alone (top row). GFP fluorescence was broadly distributed through epithelial barrier after nasal inoculation with NE (bottom left panel). These observations further confirmed the QDOT whole body distribution findings. Accordingly, in some embodiments, the invention provides that NE induces antigen uptake in a broad range of cell types (e.g., epithelial cells) in addition to uptake in professional antigen presenting cells.

### Example 5

### Phenotypic Analysis of Infiltrating Lymphocytes at the Site of Immunization

In order to characterize the local environment following immunization with an NE-based vaccine, the nasal septum (the site of immunization) was analyzed 18 hours following nasal treatment with 20% NE or after immunization with 20% NE+20 μg OVA for the presence of infiltrating leukocytes (Cd11b positive macrophages, CD11c positive dendritic cells(DC), CD45R positive B cells, and CD11b^{low} and GR-1^{high} neutrophils using flow cytometry. Results were compared to a PBS only treated control group. Increases in CD11b+ and CD11b^{low} and GR-1^{high} cell populations (macrophages and nuetrophils) were observed within the nasal septum at this time point. Thus, in some embodiments, the invention provides that immunogenic compositions (e.g., vaccines) comprising NE, when administered intranasally, recruits lymphocytes to the nasal septum.

### Example 6

### Nanoemulsion-Mediated Antigen Uptake in the Organized Nasal Associated Lymphoid Tissue

Nasopharyngeal M-cells are implicated in particulate antigen sampling in nasal compartments in mice. The ability of NE to induce translocation in these cells was characterized and compared to CT-influenced M-cell sampling. Nasal Associated Lymphoid Tissue (NALT) was qualitatively evaluated for the presence of GFP 18 hours following nasal administration (See FIG. 2C) of either NE-GFP (lower right panel), CT-GFP (lower left panel), GFP only (upper right panel) or naïve mice (upper left panel). GFP was identified in the sub-epithelial dome (SED) and along the luminal border in mice treated with GFP-NE but not in mice treated with GFP without an adjuvant. Interestingly, GFP was not detected in significant amounts in GFP-CT immunized mice. Thus, in some embodiments, the invention provides that a nanoemulsion of the invention, in sharp contrast to other adjuvants (e.g., CT), provides a material that promotes retention of one or more immunogens (e.g., protein immunogen) within the NALT and/or nasal mucosa for long periods of time (e.g., 3, 6, 12, 15, 18, 20, 24, 28, 30, 33, 36 or more hours).

Flow cytometry was used as a secondary means to confirm the presence of antigen in NALT and eliminate the possibility that tissue trafficking was antigen-specific. OVA-Alexa 647 distribution was analyzed in the NALT at 36 hours following nasal administration of OVA-Alexa 647 plus NE. A far-red fluorophore was selected to avoid any overlap in background emissions from activated macrophages. A significant increase (p=0.007) of OVA presence was observed in CD11c⁺ NALT-isolated cells in NE treated mice compared to CD11c⁺ NALT-isolated cells from naïve mice (See FIGS. 2D and E). 8.0±1.5% of CD11c expressing cells contained OVA-Alexa 647 in NE treated mice compared only 1.3±0.3% in mice treated with OVA-Alexa 647 alone. CD19⁺ cells did not contain OVA-Alexa 647 indicating a lack of B cell uptake.

### Example 7

### Nanoemulsion-mediated trans-cellular antigen uptake in ciliated epithelial cells

As shown in FIG. 2B, NE-facilitated antigen uptake was not be explained by antigen presenting cell (APC) sampling alone. Transmission electronic microscopy (TEM) was employed to further characterize the cellular and sub-cellular distribution of NE-antigen in nasal mucosa 18 hours after nasal administration of NE with quantum dots (See FIG. 3). Ciliated cells in the nasal epithelium of mice exposed to NE adjuvant contained vesicle-like material homogenously distributed throughout the cytoplasm (See FIGS. 3A, B and C). The vesicle-like structures are consistent with the appearance of early endosomes (See, e.g., Jovic et al., Histol Histopathol 2010. 25: 99-112) and not that of apoptotic bodies (See, e.g., Jones et al., Am J Physiol 1997. 273: G1174-1188). Further, they have an average diameter of 0.479 microns, consistent with the size of lipid droplets in the NE. Tight junctions in these cells remained intact (See FIG. 3C-arrows) despite the abundant vesicle-like material in the cytoplasm. In contrast, epithelial cells from untreated mice did not show the cytoplasmic vesicle-like structures (See FIG. 3F). Under higher magnification (See FIGS. 3D and E), QDOTS were detected in the cytoplasm of cells proximal to the basal lamina (See FIG. 3D—arrows) in aggregates inside the vesicle-like material, indicating that the material stayed with the NE in the cells (See FIG. 3E). Accordingly, in some embodiments, the invention provides nanoemulsion compositions and uses thereof for adjuvant-mediated enhanced antigen uptake in epithelial cells (e.g., ciliated nasal epithelial cells)). In some embodiments, the invention provides nanoemulsion compositions and uses thereof for adjuvant-mediated enhanced antigen uptake in cells other than traditional antigen presenting cells. Although an understanding of a mechanism is not needed to practice the invention and the invention is not limited to any particular mechanism of action, in some embodiments, one or more antigens/immunogens are presented to a subject's immune system via epithelial cell antigen-uptake (e.g., important for effective mucosal and/or systemic immunity (e.g., due to the presence of large numbers of epithelial cells within a site of immunization)). For example, in some embodiments, antigen delivery to epithelial cells is made possible by a nanoemulsion of the invention that in turn leads to antigen-uptake by the epithelial cells that in turn induces effective immune responses (e.g., immune responses associated with mucosal and/or systemic immunity) due to the large number of epithelial cells (e.g., significantly outnumbering antigen presenting cells) within the upper respiratory mucosa (See, e.g., Salik et al., Am J Respir Cell Mol Biot 1999. 21: 365-379).

### Example 8

### Nanoemulsion Mediates APC Activity Via Immunogenic Epithelial Cell Apoptosis and Necrosis

Having observed NE-facilitated antigen uptake in ciliated epithelial cells (See Example 7), the relevance of this activity for APC-associated antigen trafficking was examined. Although an understanding of the mechanism is not needed to practice the invention, and while the invention is not limited to any particular mechanism of action, in some embodiments, NE-loaded epithelial cells undergo apoptosis or necrosis, and are then sampled by dendritic cells (DC). In experiments conducted during development of embodiments of the invention, nasal epithelium was harvested from mice 2 hours following treatment with 15 μl 20% NE, NE without CPC (W₈₀5E), or CPC alone. This analysis was undertaken in an effort to characterize if cellular survival and/or death were influenced by the intact nanoemulsion or if individual components of nanoemulsion (e.g., detergent) mediate outcomes. The epithelium was evaluated in situ for apoptosis by staining for caspase-3 and morphologically for necrosis (See FIG. 4A). Both apoptotic (red arrows) and necrotic cells (black arrows) were identified in NE-treated epithelium, and these changes occurred without significant cellular inflammation. In contrast, tissues from mice treated with CPC alone underwent severe necrotic changes (See FIG. 4A upper right panel) with areas of complete epithelial layer disruption associated with neutrophilic infiltration (green arrows). Interestingly, mice treated with nanoemulsion without CPC (See FIG. 4A lower right panel) had similar architecture in comparison to PBS-treated controls (See FIG. 4A upper left panel), indicating a role for CPC in the induction of apoptosis. Both the apoptotic and the necrotic processes generated by NE were associated with focal disruption of tight junctions and the epithelial barrier thereby allowing para-cellular antigen infiltration into the epithelium. This process was limited in distribution and was not wide-spread.

In order to further characterize whether the observed apoptosis was immunogenic, nasal epithelial tissue sections from the above study were probed for calreticulin, an ectopically exposed protein only expressed by cells undergoing immunogenic cell death (See, e.g., Obeid et al., Cell Death Differ 2007. 14: 1848-1850). As shown in FIG. 4B, the epithelium from NE-treated mice showed markedly positive staining for calreticulin not observed in the epithelia from control animals (See FIG. 4B lower left panel—brown cells). Thus, in some embodiments, the invention provides nanoemulsion comprising detergent (e.g., CPC) that uniquely induces immunogenic epithelial cell apoptosis (e.g., that stimulates APC activity (e.g., thereby mediating the generation of immune responses associated with mucosal and/or systemic immunity (e.g., specific to antigen/immunogen co-administered with the nanoemulsion))). Accordingly, the invention also provides that subjects administered nanoemulsion lacking detergent (e.g., lacking CPC) do not display epithelial apoptosis and do not develop immune responses associated with local (e.g., mucosal) or systemic immunity to co-administered antigen.

### Example 9

### Nanoemulsion Stimulate Accessory Antigen Presenting Activity of Nasal Epithelial Cells

In order to further characterize the local effects of NE on antigen processing and presenting-related gene expression, whole nasal mucosal tissue were harvested following nasal instillation of 20% NE (15 μl) in healthy adult CD-1 mice as described in the Materials and Methods (complete microarray data has been deposited in the public database Gene Expression Omnibus (GEO) under series accession number GSE25486). Hierarchal cluster analysis of gene expression related to antigen processing and presentation was performed on the nasal mucosa using the GO term 0006955 Immune Response (See FIG. 5A). Up-regulation of MHC class I and II transcriptional expression in nasal mucosa was observed at 6 and 24 hours in NE treated mice compared to PBS controls. Thus, the invention provides, in some embodiments, compositions and methods for immune-related transcriptional activation (e.g., that accompanies NE-facilitated antigen uptake in the mucosa).

In order to determine if NE influences antigen presentation activity in nasal epithelial cells, purified cultures of primary nasal septal epithelial cells harvested from healthy adult C57BL/6N mice were examined. These cells were probed for surface expression of MCH class II after treatment with NE (0.0001%) and compared to treatment of cells exposed to 10 μg/ml CT for 12 hours or media alone as controls (See FIG. 5B). NE induced significantly more expression of MHC class II in comparison to the control groups (p=6.3×10⁻⁵). There was no significant difference in MHC class II expression between the NE and CT groups (p>0.05). Thus, the invention provides, in some embodiments, compositions and methods for inducing MHC class II expression (e.g., gene and/or protein expression) in epithelial cells (e.g., nasal epithelial cells).

### Example 10

### DEC205⁺ DC Traffic Nanoemulsion-Associated Antigen to Cervical Lymph Nodes

To determine the phenotype of APCs associated with NE-GFP in the superficial cervical LN, co-localization of GFP and either DEC205, CD19 or CD11b surface markers was determined using laser confocal microscopy of tissue harvested 18 hours following nasal administration of 10 μg GFP±20% NE (15 μl). GFP⁺ was observed to localize in DEC205⁺ cells from mice treated with NE-GFP but not in CD19 or CD11b expressing cells (See FIG. 6). Thus, the invention provides, in some embodiments, that mature DC traffic NE-associated antigen to regional lymph nodes (e.g., from epithelial cells harboring antigen delivered to a subject via nasal administration of antigen plus a nanoemulsion disclosed herein).

### Example 11

### Characterization of Innate and Adaptive Immune Responses after Nanoemulsion or CT Stimulation In Vivo

In order to characterize NE-specific effects on the innate cytokine profile of nasal tissue, RNA from whole nasal mucosal was harvested following administration of 20% NE (15 μl) in healthy adult CD-1 mice. Microarray analysis showed 2968 (1975 up-regulated and 993 down-regulated) changes in gene expression at 6 hours (complete microarray data has been deposited in the public database Gene Expression Omnibus (GEO) under series accession number GSE25486) when compared to animals administered PBS only. This data was analyzed for immune related genes (KEGG term cytokine-cytokine interaction (04060)). Expression of only 22 genes (10.5%) showed significant increases at 6 hours after nasal treatment with NE. Subsets of RNA transcripts from the pro-inflammatory cytokines/chemokines (GM-CSF, IL-1b, IL-6, KC, MCP-1, MIP-1α, RANTES, TNF, CXCL9, CXCL13 CXCL2 and CCL12) were significantly increased.

To characterize protein level changes associated with the gene expression data and to compare innate immune responses induced by NE and CT adjuvants, protein immunodetection of innate cytokines and chemokines was carried out in homogenized nasal septal tissue collected from C57BL/6 mice treated with either NE or CT. Cytokine and chemokine detection was compared to mice treated with PBS only and was evaluated using a Luminex mouse 22-plex cytokine/chemokine kit and standard ELISA (See FIGS. 7A and 7C). Mice were treated with 15 μl of either 20% NE or 1 μg CT, adjuvant doses equivalent to those used in Example 2 that generated immune responses (See FIG. 1). Nasal treatment with NE lead to increases in the cytokines G-CSF, IL-1a, IL-5, IL-6, IL-12, IP-10, TGF-β, KC and the DC maturation cytokine TSLP by 18 hours. The cytokine response profile of CT was significantly different from the NE cytokine response profile, with increases in only IL-6 and IL-12 common to both NE and CT. Thus, the invention provides, in some embodiments, nanoemulsion compositions and methods of use thereof for inducing a desired cytokine response profile (e.g., enhanced expression of G-CSF, IL-1a, IL-5, IL-6, IL-12, IP-10, TGF-β, KC and/or TSLP). Although an understanding of a mechanism is not need to practice the invention, and the invention is not limited to any particular mechanism of action, in some embodiments, induction of a desired cytokine profile associated with NE adjuvant (e.g., enhanced expression of G-CSF, IL-1a, IL-5, IL-6, IL-12, IP-10, TGF-β, KC and/or TSLP) induces NE-associated antigen specific adaptive Th17 responses (See, e.g., Bielinska et al., Crit Rev Immunol 2010. 30: 189-199) (e.g., associated with induction of both IL-6 and TGF-β (See, e.g., McGeachy et al., Nat Immunol 2007. 8: 1390-1397))).

Conventional belief in the art is that vaccine adjuvants, including CT, potentiate inflammatory cytokine and chemokine production through APC as a key step in the induction of antigen-specific immunity. However, given the ciliated epithelial cell-antigen uptake data presented above, experiments were conducted to evaluate whether non-professional antigen presenting cells, such as epithelial cells, also participate in this function. Bone marrow derived dendritic cells (BMDC) harvested from C57BL/6 mice were stimulated with a range of either NE concentrations (0.001% to 0.1%) or CT (1 μg, 10 μg, or 30 μg). Supernatants from these cells were collected and evaluated for the presence of cytokines and chemokines using the Immunex assay described herein (See FIGS. 7B and 7C). As compared to control cells, NE was found to stimulate significant production of the cytokines GM-CSF, IL-1α, IL-1β and MIP-1α. CT also stimulated IL-1α, IL-1β and MIP-1α in BMDC in addition to IL-10, G-CSF, IL-10, IL-4, IL-6, IL-9, IL-12, IL-15, IL-17, TNF-α, and KC. The only NE-stimulated cytokine significantly increased in both the nasal septum and BMDC was IL-1α. This indicates that the NE induced innate cytokine profile is unique from the cytokine profile induced by CT and that cells participating in the innate response associated with NE versus that of CT in fact differ. NE uniquely caused stromal cells (not APC's) to produce the cytokines G-CSF, IL-5, IL-6, IL-12, IP-10, KC, TGF-β and TSLP.

To confirm these results, TC-1 epithelial cells were incubated in media with either NE or CT as above. The supernatant from treated cells were evaluated with ELISA for IL-6, TGF-β and TSLP (See FIGS. 7A and 7C). Significant amounts of IL-6, TGF-β and TSLP were measured in supernatant collected from cells incubated with NE; however, only IL-6 was induced in response to CT. Thus, the invention provides, in some embodiments, that NE uniquely promotes innate cytokine and chemokine activity in epithelial cells in addition to activating APC's, whereas conventional adjuvant (e.g., CT) fails to do so.

### Example 12

### Evaluation of the Role of IL-6 Cytokine in the Adjuvant Activity of Nanoemulsion

The pro-inflammatory protein IL-6 is the most significantly detectable cytokine produced in the nasal mucosa after exposure to NE. IL-6 is involved in the induction of acute phase response and activation of both T- and B-lymphocytes (See, e.g., Vanden Bush, J Immunol 2009. 183: 4833-4837). In order further characterize the role of IL-6 in immune responses produced by NE-based immunization, mice deficient in the ability to produce IL-6 (IL-6−/−) were administered 20% NE+20 μg rPA. rPA-specific splenocyte responses from IL6−/− vaccinated mice were compared to those from WT mice (See Table 3, below). Th2 IL-5 cytokine secretion was enhanced in the IL-6−/− mice as compensatory mechanism for IL-6 deficiency. The production of Th1 and Th17 cytokines (IFN-γ, TNF-α, and IL-17) in the IL6−/− spleen cells was significantly diminished when compared to cytokines produced by WT splenocytes. Accordingly, in some embodiments, the invention provides that IL-6 is involved in NE-mediated activation and regulation of Th1 and Th17 responses in upper respiratory mucosa. Thus, although an understanding of a mechanism is not needed to practice the invention, and the present invention is not limited to any particular mechanism of action, in some embodiments, the invention provides compositions and methods for inducing expression of IL-6 that in turn activates Th1 and/or Th17 immune responses (e.g., in upper respiratory mucosa).

TABLE 3
Cytokines produced by splenocytes from IL-6 −/− or WT mice
with NE/rPA following stimulation with rPA in vitro

Fold antigen
specific stimulation^{(a)}

| Splenocyte derived-cytokines | IL-6 −/− | WT |
|---|---|---|
| Th1 type | | |
| IFN-γ | 2.2* | 14.5 |
| TNF-α | 1.5* | 23.5 |
| IL-2 | 8.8 | 2.9 |
| Th2 type | | |
| IL-6 | 1.1 | 2.4 |
| IL-4 | 3.2 | 4.0 |
| IL-5 | 60.4* | 9.0 |
| IL-10 | 2.2 | 1.6 |
| Th17 type | | |
| IL-17 | 3.91* | 58.61 |

^{(a)}Shown are the averages of ratio (stimulation plus rPA/stimulation with media without rPA) for 4 different mice.
Cytokines were detected by Luminex assay as explained in Materials and Methods.
“*” indicates statistically significant differences (p < 0.05) in cytokine expression in IL-6 −/− versus WT mice.

### Example 13

### Nanoemulsion Induces Caspase-8 Activated Immunogenic Apoptosis

Experiments conducted during development of embodiments of the invention and described herein discovered that intact nanoemulsions (not individual components) elicited immunogenic apoptosis in vivo in nasal mucosal epithelial following nasal treatment in subjects as documented by probing for calreticulin in situ (See Examples 1 and 8). In the immunogenic apoptotic pathway, early activation of the endoplasmic reticulum (ER)-sessile kinase PERK leads to phosphorylation of the translation initiation factor eIF2α, followed by partial activation of caspase-8 (but not caspase-3) and subsequent caspase-8-mediated cleavage of the ER protein BAP31 and conformational activation of Bax and Bak. Activation of Bax and Bak stimulates calreticulin that has transited the Golgi apparatus to be secreted by SNARE-dependent exocytosis (See, e.g., Panaretakis et al., EMBO, 2009, 28, 578-590). Caspase 8 is one of the first caspases activated in cells treated with FasL or TNF-α cytokine and through its interaction with other proteins regulates either cell death (apoptosis, necroptosis) or cell survival.

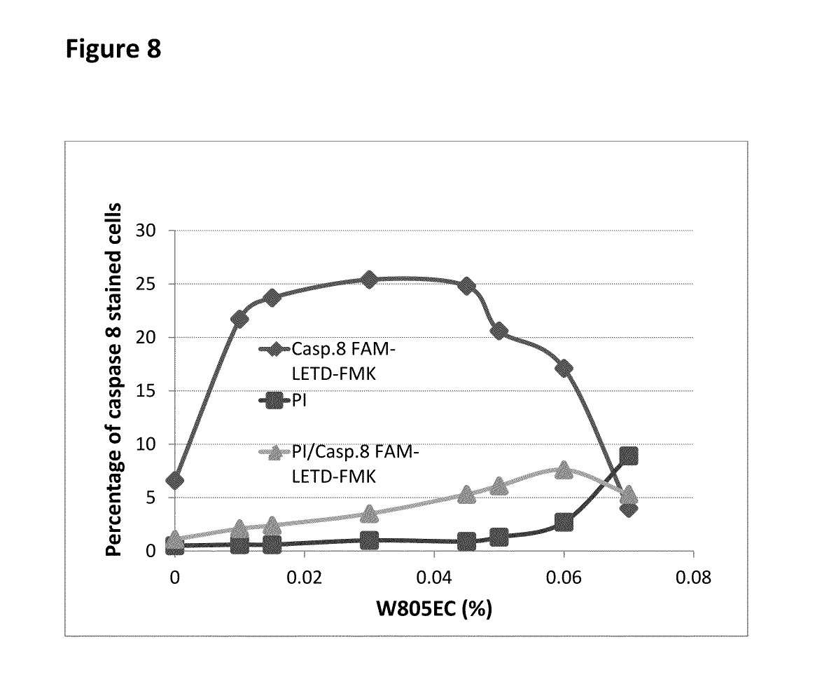

Accordingly, further experiments were conducted during development of embodiments of the invention in order to determine whether nanoemulsion adjuvants promoted immunogenic apoptosis via activation of caspase 8. Human nasal septum cells (RPMI 2650) treated with W805EC nanoemulsion were analyzed for caspase8 activity. RPMI 2650 cells were seeded at concentration of 150K/well in 1 ml of Eagle's Minimum Essential Medium supplemented with 10% FBS medium on 12-well plates. Forty-eight hours later cells were treated in situ with adding increasing concentrations of W805EC. After six-hour treatment cells were harvested and stained using Green FLICA Caspase 8 assay kit (ImmunoChemisty Technologies LLC) according to the vendor's protocol. Flow cytometry acquisition of cells was performed using Beckmann-Coulter Epics XL MCL machine. Ten thousand cells per sample were acquired and fluorescence was recorded; non-fluorescent, green fluorescent (caspase-8 positive), red fluorescent (PI positive) and double fluorescent (caspase-8/PI positive) cells were measured. Collected data were analyzed using Expo-32 software. After 6-hour treatment with increasing concentrations of NE, a NE-dependent increase in the number of cells expressing the active form of caspase 8 was observed (See FIG. 8). However, higher concentrations of NE (>0.045%) inhibited expression of active caspase 8 indicating that these cells may die due to necrosis rather than apoptosis. The increasing number of propidium iodide (PI) positive cells (See boxed line in FIG. 8) further supports this conclusion.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

What is claimed is:

1. A method of inducing immunogenic apoptosis in a subject in need thereof comprising administering to the subject an effective amount of a composition comprising a nanoemulsion.

2. The method of claim 1, wherein the subject has an infection.

3. The method of claim 1, wherein the subject is selected from the group consisting of a subject with cancer, a subject with fibrosis and a subject with a wound.

4. The method of claim 1, wherein inducing immunogenic apoptosis comprises activation of caspase 8 in the subject.

5. The method of claim 4, wherein activation of caspase 8 results in calreticulin expression in the subject

6. The method of claim 4, wherein the immunogenic apoptosis induces innate and/or adaptive immune responses that occur in the absence of inflammation.

7. The method of claim 6, wherein the innate and/or adaptive immune responses take place in the absence epithelial disruption.

8. The method of claim 1, wherein the nanoemulsion induces antigen uptake and trafficking via ciliated epithelial cells.

9. The method of claim 8, wherein antigen trafficking via ciliated epithelial cells target antigens to dendritic cells within regional and/or draining lymph nodes.

10. The method of claim 9, wherein antigen targeted dendritic cells recruit lymphocytes to regional and/or draining lymph nodes and subsequent polarization toward a Th1/Th17 immune response.

11. The method of claim 10, wherein polarization toward a Th1/Th17 immune response prevents the onset of infection.

12. The method of claim 8, wherein antigen-loaded ciliated epithelial cells interact directly with antigen specific lymphocytes in sinonasal epithelium leading to local and systemic immune responses.

13. The method of claim 5, wherein the immune responses comprise induction and/or expression of cytokines ganulocyte-macrophage colony-stimulating factor (GM-CSF), IL-6, IL-1a, IL-1b and MIP1α and does not comprise induction and/or expression of the cytokines IL-4 or TNF-α.

14. The method of claim 13, wherein the induction and/or expression of cytokines occurs through activated epithelial cells.

15. The method of claim 1, wherein the nanoemulsion comprises a positive surface charge.

16. The method of claim 1, wherein the nanoemulsion comprises cetylpyridinium chloride (CPC) and an organic solvent.

17. The method of claim 16, wherein the organic solvent is ethanol.

18. The method of claim 1, wherein administering to the subject occurs via administration to a mucosal surface.

19. A method of inducing caspase 8 activation in a subject comprising administering to a subject in need thereof an effective amount of a composition comprising a nanoemulsion.

20. The method of claim 19, wherein the subject is selected from the group consisting of a subject with cancer, a subject with fibrosis and a subject with a wound.
